(19) United States
(12) Patent Application Publication
(10) Pub. No.: US 2025/0122202 A1
(43) Pub. Date: Apr. 17, 2025

(54) EGFR INHIBITOR, PREPARATION METHOD THEREFOR AND USE THEREOF

(71) Applicant: SHANDONG NEW TIME PHARMACEUTICAL CO., LTD., Linyi City, ShangDong Province (CN)

(72) Inventors: Guimin Zhang, Linyi City, ShangDong Province (CN); Hao Zhang, Linyi City, ShangDong Province (CN); Jingchun Yao, Linyi City, ShangDong Province (CN); Jiang Yuan, Linyi City, ShangDong Province (CN); Guifang Zhao, Linyi City, ShangDong Province (CN); Rui Li, Linyi City, ShangDong Province (CN); Guangyan Li, Linyi City, ShangDong Province (CN); Yun Zhao, Linyi City, ShangDong Province (CN); Hongbao Liang, Linyi City, ShangDong Province (CN); Zhenjun Zhang, Linyi City, ShangDong Province (CN); Xiangxia Zhu, Linyi City, ShangDong Province (CN)

(21) Appl. No.: 18/692,470

(22) PCT Filed: Sep. 16, 2022
(86) PCT No.: PCT/CN2022/119451

§ 371 Date: Mar. 15, 2024

(30) Foreign Application Priority Data
Sep. 18, 2021 (CN) 20111117969.3
Mar. 2, 2022 (CN) 202210197158.7
Mar. 2, 2022 (CN) 202210197277.2

(51) Int. Cl. C07D 487/04; A61K 31/519

(52) U.S. Cl. CPC C07D 487/04; A61K 31/519

(57) ABSTRACT

Disclosed are a compound

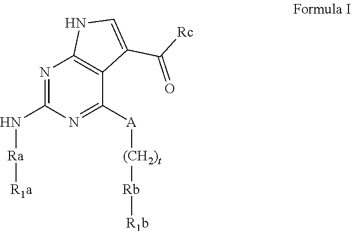

of formula I or a pharmaceutically acceptable salt thereof, and use thereof in regulating and controlling an EGFR tyrosine kinase activity or preventing and treating an EGFR-related disease. The EGFR inhibitor of formula I has an inhibitory activity on EGFR D770-N771 ins NPG and NPG/T790M kinases, and an inhibitory effect on an EGFR-Del19/T790M/C797S kinase and cell proliferation of a KC-0122:Ba/F3EGFR-L858R/T790M/C797S three-mutation cell line and a KC-0116:Ba/F3EGFR-Del19/T790M/C797S three-mutation cell line.

### TECHNICAL FIELD

The present invention relates to the field of pharmaceutical chemistry and pharmacotherapeutics, and an EGFR inhibitor and a preparation method therefor, and further relates to use of the compound in preparing an anti-cancer drug.

### BACKGROUND ART

EGFR mutation mainly occurs in No. 18-21 exons, wherein deletion mutation of No. 19 exon (Del19) and L858R point mutation of No. 21 exon are the most common EGFR mutation subtypes, account for 90% of all mutation types, and are called sensitive mutation of the EGFR gene. The first generation of EGFR inhibitors (gefitinib, erlotinib, and icotinib) are developed for the EGFR mutation. However, after a majority of patients with non-small cell lung cancer (NSCLC) take the medicines, they will develop an acquired drug resistance within 6-12 months. T790M mutation is the primary cause of the drug resistance.

To overcome the T790M drug resistance, researchers develop second-generation EGFR inhibitors such as afatinib, dacomitinib, and neratinib. However, the second-generation inhibitors have poor selectivity, also show strong inhibitory activity on wild-type EGFR (EGFR-WT), have low maximum tolerated dose and narrow therapeutic window, and are therefore limited in clinical use.

In order to overcome the problem of poor selectivity of the second-generation EGFR-TKIs, third-generation EGFR inhibitors represented by osimertinib are successfully developed. However, the acquired resistance of the third-generation inhibitors is still unavoidable. EGFR-C797S mutation accounts for about 20% of the many resistance mechanisms and is one of the major resistance mechanisms. When the C797S mutation occurs in cis, no medicine is available clinically at present. Therefore, overcoming the drug resistance of C797S and developing fourth-generation EGFR-TKIs with high efficiency and low toxicity become the consensus of domestic and foreign counterparts, and are also the key points and difficulties in the research and development of new drugs for NSCLC. At present, although the fourth-generation EGFR small molecules are reported successively, most of them are in a preclinical research stage, few compounds enter clinical trials, and no medicines are on the market.

It is of great clinical significance and application prospect to research and develop a drug-resistant novel fourth-generation EGFR inhibitor for selectively inhibiting the C797S mutation.

### SUMMARY

The present invention discloses an EGFR inhibitor, a preparation method for and a medical use thereof. The method for preparing the inhibitor in the present invention has a mild and easily-controlled condition, convenient and simple post-treatment, and also practicability and universality at the same time. Pharmacological experiment results show that the compound of the present invention has a significant inhibitory activity on EGFR D770-N771 ins NPG and EGFR D770-N771 ins NPG/T790M kinases. The compound has an excellent inhibitory activity on EGFR-Del19/T790M/C797S kinases, an inhibitory activity on a KC-0122:Ba/F3 EGFR-L858R/T790M/C797S three-mutation cell line and a KC-0116:Ba/F3EGFR-Del19/T790M/C797S three-mutation cell line, and has a stronger inhibitory activity on single-mutation and double-mutation cells at the same time.

An objective of the present invention is to provide a compound show in formula I, a pharmaceutically acceptable salt thereof, or a stereoisomer thereof,

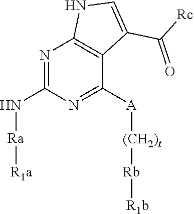

L wherein the Rc is

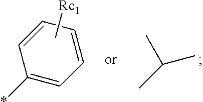

the number of the Rc₁ is 0, 1, 2, or 3; each Rc₁ is independently selected from at least one of halogen and C₁₋₃ alkyl;
the Ra is a substituted or unsubstituted phenyl, or a substituted or unsubstituted

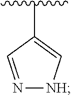 [SMILES: *c1cn[nH]c1]

the substituents are each independently selected from halogen, C₁₋₃ alkyl, and C₁₋₃ alkoxy; the number of the respective substituents of the phenyl or the

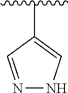 [SMILES: *c1cn[nH]c1]

is 0, 1, or 2; the halogen is F, Cl, Br, and I;
the R₁a is selected from H,

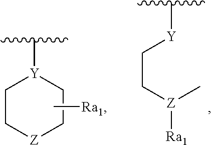

or C₁₋₃ alkyl; the Y and the Z are each independently selected from N, O, and C, wherein the N and the C are substituted by 0 or 1 C₁₋₃ alkyl and the insufficient valence bonds are complemented by H; and the Ra₁ is H or C₁₋₃ alkyl;
the A is O or N, wherein the N is substituted by at least one of H and C₁₋₃ alkyl;
the t is selected from 0, 1, 2, or 3;
the Rb is selected from groups in one or more groups of the following 1)-5):
1) a substituted or unsubstituted 5- to 8-membered spiroheterocycle, wherein a heteroatom in the spiroheterocycle is O, S, or N; the substituent of the spiroheterocycle is one or more independent substituted or unsubstituted amino, C₁₋₃ alkyl, C₁₋₃ alkoxy, and OH; and the substituent of the amino is H or one or two C₁₋₃ alkyls;
2) a substituted or unsubstituted C₈-C₁ bridge ring, wherein the substituent of the bridge ring is one or more independent substituted or unsubstituted amino, C₁₋₃ alkyl, C₁₋₃ alkoxy, and OH; and the substituent of the amino is H or one or two C₁₋₃ alkyls;
3) a substituted or unsubstituted amino, wherein the substituent of the amino is H or one or two C₁₋₃ alkyls, or —(CH₂)ₐ-E-(CH₂)_{b}—, and N in the amino is linked with both ends of the —(CH₂)ₐ-E-(CH₂)_{b}— to form a ring; the E is C or O, and the a is 1, 2, 3, or 4; and the b is 1, 2, 3, or 4;
4)

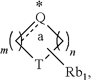

wherein the number of the double bonds a is 0, 1, 2, or 3; the position of the a is any position with a reasonable valence bond in the ring; the insufficient valence bonds are complemented by H;
the m is 0, 1, 2, or 3;
the n is 0, 1, 2, or 3;
the Q and the T are each independently N, O, S, or C; and the insufficient valence bonds are complemented by H; and
5)

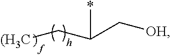

wherein the f is 1, 2, or 3; and the h is 0, 1, or 2;
the number of the Rb₁ is 0, 1, or 2; the Rb₁ is amino, hydroxy, C₁₋₃ alkyl, C₁₋₃ alkoxy, C₁₋₃ alkyl substituted by hydroxy, SO₂R₂b,

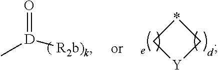

the R₂b is a substituted or unsubstituted C₁₋₃ alkyl, a substituted or unsubstituted phenyl, C₃₋₆ cycloalkyl, C₂₋₅ alkenyl, or

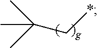

the substituent of the C₁₋₃ alkyl or the phenyl is halogen or hydroxyl; the D is selected from C, P, or S; the g is 1, 2, or 3; the e and the d are each independently selected from 0, 1, 2, or 3; and the Y is selected from C, O, or N; and
the R₁b is H, NH₂, hydroxyl, C₁₋₃ alkoxy, C₁₋₃ alkyl substituted by hydroxyl, SO₂R₂b, C₁₋₃ alkyl,

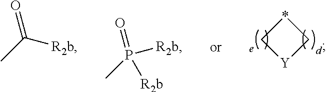

each R₂b is independently a substituted or unsubstituted C₁₋₃ alkyl, a substituted or unsubstituted phenyl, C₃₋₆ cycloalkyl, C₂₋₅ alkenyl, or

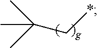

the substituent of the C₁₋₃ alkyl or the phenyl is halogen or hydroxyl; the g is 1, 2, or 3; the e and the d are each independently selected from 0, 1, 2, or 3; the Y is selected from C, O, or N; the C₁₋₃ alkyl is methyl, ethyl, propyl, or isopropyl; the C₁₋₃ alkoxy is methoxy, ethoxy, propoxy, or isopropoxy; and the halogen is F, Cl, Br, or I.

In some examples, the compound shown in formula I, a stereoisomer thereof, or a pharmaceutically acceptable salt thereof, wherein the substituent Rc thereof is selected from any one of the following groups:
the number of the Rc₁ is 0, 1, 2, or 3; each Rc₁ is independently selected from at least one of F, Cl, Br, I, and C₁₋₃ alkyl;
the Rc is

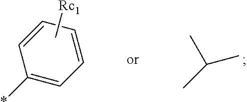

the number of the Rc₁ is 1, 2, or 3; and each Rc₁ is independently selected from at least one of F, Cl, Br, methyl, ethyl, and propyl;
the Rc is

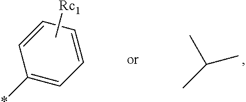

and the number of the Rc₁ is 0, 1, 2, or 3; and each Rc₁ is independently selected from at least one of F, Cl, and C₁₋₃ alkyl;
the Rc is

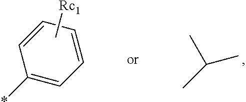

and the number of the Rc₁ is 0, 1, 2, or 3; and each Rc₁ is independently selected from at least one of F, Cl, and methyl;
the Rc is

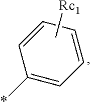

and the number of the Rc₁ is 0, 1, 2, or 3; each Rc₁ is independently selected from at least one of F and C₁₋₃ alkyl; and further the C₁₋₃ alkyl is selected from methyl or ethyl;
the Rc is

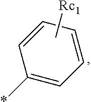

and the number of the Rc₁ is 0, 1, 2, or 3; and each Rc₁ is independently selected from at least one of F, Cl, and methyl;
the Rc is

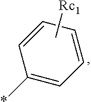

and the number of the Rc₁ is 1, 2, or 3; and each Rc₁ is independently selected from at least one of F, Cl, and methyl;
the Rc is

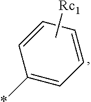

and the number of the Rc₁ is 0, 1, or 3; and each Rc₁ is independently selected from at least one of F and methyl;
the Rc is

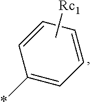

and the number of the Rc₁ is 1 or 3; and each Rc₁ is independently selected from at least one of F;
the Rc is

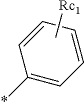

and the Rc₁ is a single F;
the Rc is

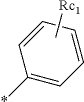

and the Rc₁ is F; and the number of the Rc₁ is 3, wherein at least two are respectively at the ortho-position and para-position of the benzene ring;
the Rc is

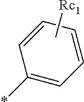

and the Rc₁ is F; and the number of the Rc₁ is 0, 1, or 3;
the Rc is

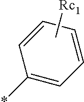

and the number of the Rc₁ is 0 or the Rc₁ is 1 F or 3 Fs or one methyl;
the Rc is

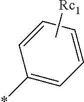

and the number of the Rc₁ is 0 or the Rc₁ is 1 F or 3 Fs or one methyl, and the preferable position is the para-position of the benzene ring;
the Rc is

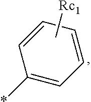

and the number of the Rc₁ is 1, 2, or 3; and each Rc₁ is independently selected from at least one of F and Cl;
the Rc is

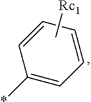

and the number of the Rc₁ is 1 or 2; and each Rc₁ is independently selected from at least one of F and Cl;
the Rc is

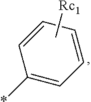

,and the number of the Rc₁ is 1 or 2; and each Rc₁ is independently selected from F or a combination of F and Cl;
the Rc is

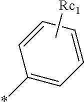

and the Rc₁ is 1 or 2 or 3 Fs;
the Rc is

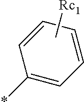

and the Rc₁ is 3 Fs;
the Rc is

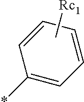

and the Rc1 is 2 Fs; and
the Rc is

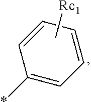

and the Rc1 is 1 F and at the para-position of the benzene ring.

In some examples, the compound shown in formula I, a stereoisomer thereof, or a pharmaceutically acceptable salt thereof, the substituent Ra thereof is selected from any one of the following groups:
the Ra is a substituted or unsubstituted phenyl, or a substituted or unsubstituted

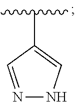 [SMILES: *c1cn[nH]c1]

the substituents are each independently selected from F, C₁₋₃ alkyl, and C₁₋₃ alkoxy; the number of the respective substituents of the phenyl or the

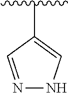 [SMILES: *c1cn[nH]c1]

is 0, 1, or 2; the halogen is F, Cl, Br, and I; the substituent of the phenyl or the

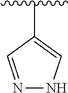 [SMILES: *c1cn[nH]c1]

in the Ra is F, Cl, C₁₋₃ alkyl, and C₁₋₃ alkoxy; and the number of the respective substituents of the phenyl or the

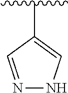 [SMILES: *c1cn[nH]c1]

is 0, 1, or 2;
each substituent of the phenyl or the

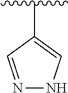 [SMILES: *c1cn[nH]c1]

in the Ra is independently selected from F, Cl, methyl, ethyl, methoxy, and ethoxy; and the number of the respective substituents of the phenyl or the

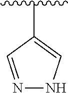 [SMILES: *c1cn[nH]c1]

is 0 or 1;
the substituent of the phenyl or the

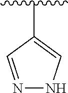 [SMILES: *c1cn[nH]c1]

in the Ra is F, methyl, and methoxy; and the number of the respective substituents of the phenyl or the

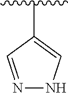 [SMILES: *c1cn[nH]c1]

is 0 or 1;
the Ra is phenyl;
the substituent of the phenyl or the

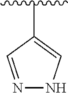 [SMILES: *c1cn[nH]c1]

in the Ra is F, methyl, and methoxy; and the number of the respective substituents of the phenyl or the

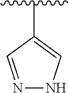 [SMILES: *c1cn[nH]c1]

is 1; and
the Ra is a substituted or unsubstituted phenyl, or a substituted or unsubstituted

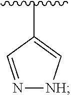 [SMILES: *c1cn[nH]c1]

the substituent of the phenyl or the

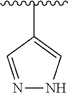 [SMILES: *c1cn[nH]c1]

in the Ra is methyl; and the number of the respective substituents of the phenyl or the

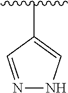 [SMILES: *c1cn[nH]c1]

is 1.

In some examples, the compound shown in formula I, a stereoisomer thereof, or a pharmaceutically acceptable salt thereof, wherein the substituent R₁a thereof is selected from any one of the following groups:
the R₁a is selected from H,

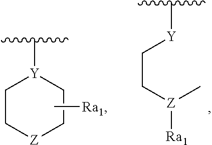

or C₁₋₃ alkyl; the Y and the Z are each independently selected from N, O, and C, and the insufficient valence bonds are complemented by H; and the Ra₁ is H or C₁₋₃ alkyl;
the R₁a is selected from H,

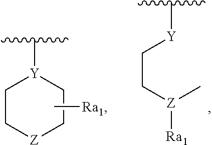

or C₁₋₃ alkyl; the Y is C, O, or N; the Z is N or O, wherein the N is substituted by 0 or 1 C₁₋₃ alkyl, and the insufficient valence bonds are complemented by H; and the Ra₁ is H or C₁₋₃ alkyl;
the R₁a is selected from H,

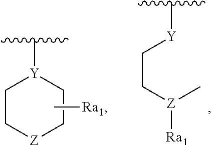

or C₁₋₃ alkyl; the Y is C, O, or N; and the Z is N or O, wherein the N is substituted by 0 or 1 C₁₋₃ alkyl and the insufficient valence bonds are complemented by H;
the R₁a is selected from H,

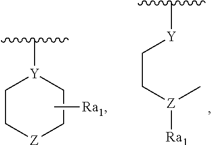

or C₁₋₃ alkyl; the Y is C or N; the Z is N or O, wherein the N is substituted by 0 or 1 methyl and the insufficient valence bonds are complemented by H; and the Ra₁ is H, CH₃, or CH₂CH₃;
the R₁a is selected from H or

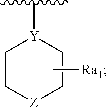

the Y is N; the Z is N or O; the insufficient valence bonds are complemented by H; and the Ra₁ is H or CH₃;
the R₁a is selected from

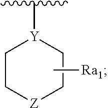

the Y is N; the Z is N or O; the insufficient valence bonds are complemented by H; and the Ra₁ is H or CH₃;
the R₁a is selected from H,

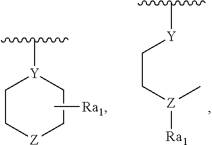

or C₁₋₃ alkyl; the Y is C or N; the Z is N or O; and the Ra₁ is H or C₁₋₃ alkyl;
the R₁a is selected from H,

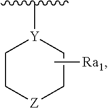

C₁₋₃ alkyl; the Y is N; the Z is N or O; and the Ra₁ is H or methyl;
the R₁a is selected from H,

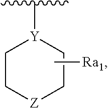

or C₁₋₃ alkyl; the Y is C or N; the Z is N; and the Ra₁ is H or methyl; and
the R₁a is selected from

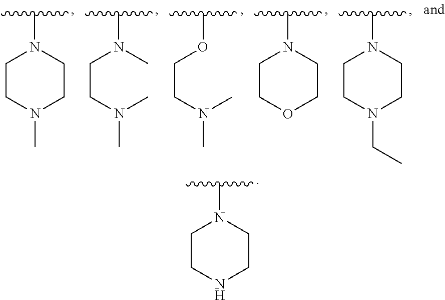

In some examples, the compound shown in formula I, a stereoisomer thereof, or a pharmaceutically acceptable salt thereof, wherein the substituent R₁a thereof is selected from any one of the following groups:
the Ra₁ is H or C₁₋₃ alkyl;
the Ra₁ is H, CH₃, or CH₂CH₃;
the Ra₁ is CH₃ or CH₂CH₃;
the Ra₁ is methyl; and
the Ra₁ is ethyl.

In some examples, the compound shown in formula I, a stereoisomer thereof, or a pharmaceutically acceptable salt thereof, wherein the substituent A thereof is selected from any one of the following groups:
the A is O or N, wherein the N is substituted by at least one of H and C₁₋₃ alkyl;
the A is O or N, wherein the N is substituted by at least one of H or methyl;
the A is O or N, wherein the N is substituted by H; and
the A is N, wherein the N is substituted by H.

In some examples, the compound shown in formula I, a stereoisomer thereof, or a pharmaceutically acceptable salt thereof, wherein the t is selected from any one of the following groups:
the t is selected from 0, 1, or 2;
the t is selected from 0 or 2;
the t is selected from 0;
the t is selected from 1;
the t is selected from 2; and
the t is selected from 3.

In some examples, the compound shown in formula I, a stereoisomer thereof, or a pharmaceutically acceptable salt thereof, wherein the number of the Rb₁ is 0, 1, or 2; the Rb₁ is amino, hydroxy, C₁₋₃ alkyl, C₁₋₃ alkoxy, C₁₋₃ alkyl substituted by hydroxy, SO₂R₂b,

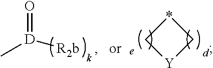

each R₂b is independently a substituted or unsubstituted C₁₋₃ alkyl, a substituted or unsubstituted phenyl, C₃₋₆ cycloalkyl, C₂₋₅ alkenyl, or

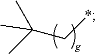

the substituent of the C₁₋₃ alkyl or the phenyl is halogen or hydroxyl; the D is selected from C or P; the k is 1 or 2; the g is 1, 2, or 3; the e and the d are each independently selected from 0, 1, 2, or 3; and the Y is selected from C, O, or N.

In some examples, the compound shown in formula I, a stereoisomer thereof, or a pharmaceutically acceptable salt thereof, wherein the number of the Rb₁ is 0, 1, or 2; the Rb₁ is amino, hydroxy, C₁₋₃ alkyl, C₁₋₃ alkoxy, or C₁₋₃ alkyl substituted by hydroxy.

In some examples, the compound shown in formula I, a stereoisomer thereof, or a pharmaceutically acceptable salt thereof, wherein the Rb is selected from groups in one or more groups of the following 1)-5):
1) a substituted or unsubstituted 5- to 8-membered spiroheterocycle, wherein a heteroatom in the spiroheterocycle is O;
2) a substituted or unsubstituted 10 bridge ring, wherein the substituent of the bridge ring is one or more independent amino, C₁₋₃ alkyl, C₁₋₃ alkoxy, and OH;
3) a substituted or unsubstituted amino, wherein the substituent of the amino is one or two C₁₋₃ alkyls, or —(CH₂)ₐ-E-(CH₂)_{b}—, and N in the amino is linked with both ends of the —(CH₂)ₐ-E-(CH₂)_{b}— to form a ring; the E is C or O, and the a is 1, 2, or 3; and the b is 1, 2, or 3;
4)

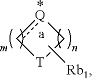

wherein the number of the double bonds a is 0, 1, 2, or 3; the position of the a is any position with a reasonable valence bond in the ring; the insufficient valence bonds are complemented by H; the m is 0, 1, 2, or 3; the n is 0, 1, 2, or 3; the Q and the T are each independently N, O, S, or C; and the insufficient valence bonds are complemented by H; and
5)

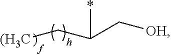

wherein the f is 1, 2, or 3; and the h is 0 or 1; and
the number of the Rb₁ is 0, 1, or 2; the Rb₁ is amino, hydroxy, C₁₋₃ alkyl, C₁₋₃ alkoxy, C₁₋₃ alkyl substituted by hydroxy, SO₂R₂b,

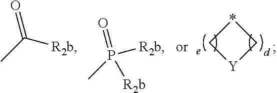

each R₂b is independently a substituted or unsubstituted C₁₋₃ alkyl, a substituted or unsubstituted phenyl, C₃₋₆ cycloalkyl, C₂₋₅ alkenyl, or

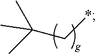

the substituent of the C₁₋₃ alkyl or the phenyl is halogen or hydroxyl; the g is 1, 2, or 3; the e and the d are each independently selected from 0, 1, 2, or 3; and the Y is selected from C, O, or N.

In some examples, the compound shown in formula I, a stereoisomer thereof, or a pharmaceutically acceptable salt thereof, wherein the Rb is selected from

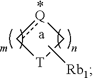

the number of the double bonds a is 0, 1, 2, or 3; the position of the a is any position with a reasonable valence bond in the ring; the insufficient valence bonds are complemented by H; the m is 0, 1, 2, or 3; the n is 0, 1, 2, or 3; the Q and the T are each independently N, O, S, or C; and the insufficient valence bonds are complemented by H.

In some examples, the compound shown in formula I, a stereoisomer thereof, or a pharmaceutically acceptable salt thereof, wherein the number of the Rb₁ is 0 or 1; the Rb₁ is amino, hydroxy, hydroxymethyl, C₁₋₃ alkyl, C₁₋₃ alkoxy, C₁₋₃ alkyl substituted by hydroxy, SO₂R₂b,

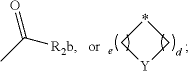

each R₂b is independently a substituted or unsubstituted C₁₋₃ alkyl, cyclopropyl, or C₂₋₃ alkenyl; the substituent of the C₁₋₃ alkyl is halogen or hydroxyl; the e and the d are each independently selected from 0, 1, 2, or 3; and the Y is selected from C, O, or N.

In some examples, the compound shown in formula I, a stereoisomer thereof, or a pharmaceutically acceptable salt thereof, wherein the number of the Rb₁ is 0, 1, or 2; and the Rb₁ is selected from any one of the following groups:
the Rb₁ is amino, hydroxy, C₁₋₃ alkyl, C₁₋₃ alkoxy, or C₁₋₃ alkyl substituted by hydroxy;
the Rb₁ is C₁₋₃ alkyl or C₁₋₃ alkoxy;
the Rb₁ is amino, hydroxy, C₁₋₃ alkyl, C₁₋₃ alkoxy, or C₁₋₃ alkyl substituted by hydroxy;
the Rb₁ is amino, hydroxy, or C₁₋₃ alkyl substituted by hydroxy;
the Rb₁ is amino or hydroxymethyl;
the Rb₁ is hydroxymethyl;
the Rb₁ is amino;
the Rb₁ is hydroxy;
the Rb₁ is hydroxyethyl; and
the Rb₁ is methyl, ethyl, or propyl.

In some examples, the compound shown in formula I, a stereoisomer thereof, or a pharmaceutically acceptable salt thereof, wherein the Rb is selected from any one of the following groups:
the Rb is

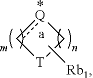

wherein the Q and the T are both C; the a is 0 and the insufficient valence bonds are complemented by H; the m is 2; and the n is 2;
the Rb is

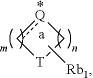

wherein the Q is C; the T is C and O; the a is 0 or 1 and the insufficient valence bonds are complemented by H; the m is 1, 2, or 3; and the n is 0, 1, or 2;
the Rb is

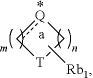

wherein the Q is C; the T is O or N; the a is 0 and the insufficient valence bonds are complemented by H; the m is 2 or 3; and the n is 1 or 2;
the Rb is

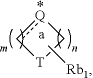

wherein the Q is C; the T is O; the a is 0 and the insufficient valence bonds are complemented by H; the m is 3; and the n is 1;
the Rb is

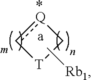

wherein the Q and the T are both C; the a is 0 and the insufficient valence bonds are complemented by H; the m is 1; and the n is 1;
the Rb is

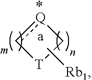

wherein the Q and the T are both C; the a is 1 and the insufficient valence bonds are complemented by H; the m is 2; and the n is 1;
the Rb is

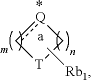

wherein the Q is C; the T is C; the a is 0 and the insufficient valence bonds are complemented by H; the m is 1; and the n is 0;
the Rb is

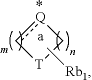

wherein the Q is C or N; the T is C, O, or N; the a is 0, 1, or 3 and the insufficient valence bonds are complemented by H; the m is 1, 2, or 3; and the n is 1 or 2;
the Rb is

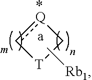

wherein the Q is C; the T is N; the a is 0 and the insufficient valence bonds are complemented by H; the m is 2 or 3; and the n is 1;
the Rb is

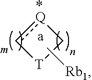

wherein the Q is C; the T is O or N; the a is 0 and the insufficient valence bonds are complemented by H; the m is 3; and the n is 1;
the Rb is

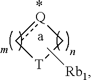

wherein the Q is C; the T is O or N; the a is 0 and the insufficient valence bonds are complemented by H; the m is 2; and the n is 2;
the Rb is

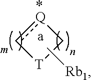

wherein the Q is C; the T is C or O; the a is 0 and the insufficient valence bonds are complemented by H; the m is 2; and the n is 2;
the Rb is

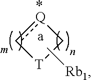

wherein the Q is C; the T is C, O, or N; the a is 0 and the insufficient valence bonds are complemented by H; the m is 2 or 3; and the n is 1 or 2;
the Rb is

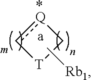

wherein the Q is C; the T is C or N; the a is 0 and the insufficient valence bonds are complemented by H; the m is 2 or 3; and the n is 1 or 2; and
the Rb is

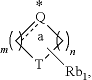

wherein the Q is C or N; the T is C, O, or N; the a is 0, 1, or 3 and the insufficient valence bonds are complemented by H; the m is 1, 2, or 3; and the n is 1 or 2.

In some examples, the compound shown in formula I, a stereoisomer thereof, or a pharmaceutically acceptable salt thereof, wherein the R₁b is selected from any one of the following groups: the R₁b is H, NH₂, hydroxyl, C₁₋₃ alkoxy, C₁₋₃ alkyl substituted by hydroxyl, SO₂R₂b, C₁₋₃ alkyl,

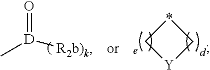

each R₂b is independently a substituted or unsubstituted C₁₋₃ alkyl, a substituted or unsubstituted phenyl, C₃₋₆ cycloalkyl, C₂₋₅ alkenyl, or

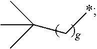

the substituent of the C₁₋₃ alkyl or the phenyl is halogen or hydroxyl; the D is selected from C or P; the k is 1 or 2; the g is 1, 2, or 3; the e and the d are each independently selected from 0, 1, 2, or 3; and the Y is selected from C, O, or N;
the R₁b is H, NH₂, hydroxymethyl, hydroxyethyl, hydroxypropyl, methyl, ethyl, propyl, isopropyl, OH, SO₂R₂b, ((CH₃)₂CH₂,

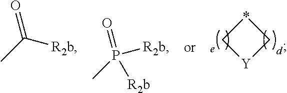

each R₂b is independently a substituted or unsubstituted C₁₋₃ alkyl, a substituted or unsubstituted phenyl, C₃₋₅ cycloalkyl, C₂₋₃ alkenyl, or

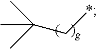

the substituent of the phenyl is halogen or hydroxyl; the D is selected from C, P, or S; the k is 1 or 2; the g is 1, 2, or 3; and the g is 1, 2, or 3;
the R₁b is H, NH₂, hydroxymethyl, hydroxyethyl, hydroxypropyl, methyl, ethyl, propyl, isopropyl, OH, SO₂R₂b, ((CH₃)₂CH₂,

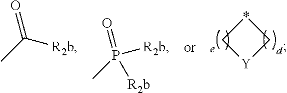

each R₂b is independently a substituted or unsubstituted C₁₋₃ alkyl, a substituted or unsubstituted phenyl, C₃₋₅ cycloalkyl, C₂₋₃ alkenyl, or

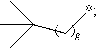

the substituent of the phenyl is halogen or hydroxyl; the D is selected from C or P; the k is 1 or 2; the g is 1, 2, or 3; and the g is 1, 2, or 3;
the R₁b is H, NH₂, HOCH₂, OH, SO₂R₂b, CH₃, CH₃CH₂, (CH₃)₂CH₂,

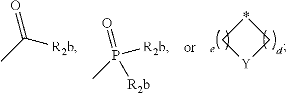

each R₂b is independently a substituted or unsubstituted C₁₋₃ alkyl, cyclopropyl, cyclobutyl, cyclopentyl, cyclooctyl, ethenyl, propenyl, or

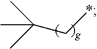

and the g is 1, 2, or 3;
the R₁b is H, NH₂, HOCH₂, OH, SO₂R₂b, CH₃, CH₃CH₂, (CH₃)₂CH₂,

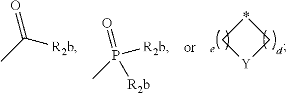

each R₂b is independently methyl, ethyl, propyl, isopropyl, hydroxymethyl, hydroxyethyl, hydroxypropyl, cyclopropyl, cyclobutyl, cyclopentyl, cyclooctyl, ethenyl, propenyl, or

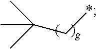

and the g is 1, 2, or 3;
the R₁b is H, amino, hydroxy, C₁₋₃ alkyl substituted by hydroxy, C₁₋₃ alkoxy,

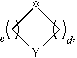

or SO₂R₂b, and each R₂b is independently selected from C₁₋₃ alkyl or C₃₋₆ cycloalkyl; and the e and the d are each independently selected from 1, 2, or 3; and the Y is selected from C, N, or O;
the R₁b is H, amino, hydroxy, C₁₋₃ alkyl, C₁₋₃ alkyl substituted by hydroxy, C₁₋₃ alkoxy, or SO₂R₂b, and each R₂b is independently selected from C₁₋₃ alkyl;
the Rb₁ is H, amino, hydroxy, C₁₋₃ alkyl, C₁₋₃ alkoxy, or C₁₋₃ alkyl substituted by hydroxy;
the Rb₁ is H, amino, hydroxy, or C₁₋₃ alkyl substituted by hydroxy; and
the Rb₁ is H, amino, or C₁₋₃ alkyl substituted by hydroxy.

In some examples, the compound shown in formula I, a stereoisomer thereof, or a pharmaceutically acceptable salt thereof, wherein the C₁₋₃ alkyl is methyl, ethyl, propyl, or isopropyl; the C₁₋₃ alkoxy is methoxy, ethoxy, propoxy, or isopropoxy; and the halogen is F, Cl, Br, or I.

In some examples, the compound shown in formula I, a stereoisomer thereof, or a pharmaceutically acceptable salt thereof, wherein the C₁₋₃ alkyl is methyl and ethyl.

In some examples, the compound shown in formula I, a stereoisomer thereof, or a pharmaceutically acceptable salt thereof, wherein the C₁₋₃ alkoxy is methoxy, ethoxy, propoxy, or isopropoxy

In some examples, the compound shown in formula I, a stereoisomer thereof, or a pharmaceutically acceptable salt thereof, wherein the halogen is F, Cl, Br, or I.

In some examples, the compound shown in formula I, a stereoisomer thereof, or a pharmaceutically acceptable salt thereof, wherein the halogen is F or/and Cl.

In some examples, the Rc₁ is at least one of H, F, Cl, and CH₃.

In some examples, the number of the Rc₁ is 1, 2, or 3.

In some examples, the position of the Rc₁ is one, two, or three of the positions 2, 3, 4, 5, and 6 on the benzene ring.

In some examples, the substituent of phenyl or

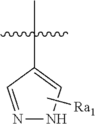

of the Ra is F and CH₃O—.

In some examples, the Rb is a substituted or unsubstituted 5- to 8-membered spiroheterocycle, a substituted or unsubstituted C₈-C₁₁ bridge ring, a substituted or unsubstituted amino,

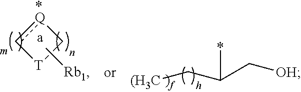

a heteroatom in the spiroheterocycle is O, S, or N; the substituent of the spiroheterocycle or the bridge ring is one or more independent NH₂, C₁₋₃ alkyl, C₁₋₃ alkoxy, and OH; and the substituent of the amino is one or two C₁₋₃ alkyls; the m is 0, 1, 2, or 3; the n is 0, 1, 2, or 3; the Q and the T are each independently N, O, S, or C; the insufficient valence bonds are complemented by H; the f is 1, 2, or 3; and the h is 0, 1, or 2.

In some examples, the f is 2 or 3.

In some examples, the Rb₁ is methyl.

In some examples, each R₂b is independently methyl, cyclopropyl, ethenyl, and 2,2,2-trimethyl-ethyl.

In some examples, the Rb is a substituted or unsubstituted 5- to 8-membered spiroheterocycle, a substituted or unsubstituted C₈-C₁₁ bridge ring, a substituted or unsubstituted amino,

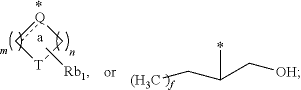

a heteroatom in the spiroheterocycle is O, S, or N; the substituent of the spiroheterocycle or the bridge ring is one or more NH₂, HOCH₂, OH, and CH₃; and the substituent of the amino is one or two C₁₋₃ alkyls,

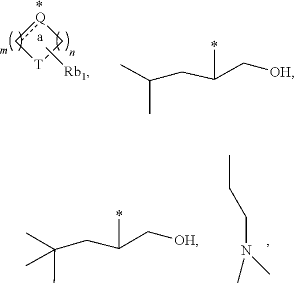

and a substituted or unsubstituted 7-membered spiroheterocycle or C₁₀ bridge ring.

In some examples, the Rb is

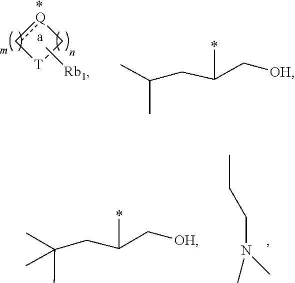

and substituted or unsubstituted 3-oxaspiro[3,3]heptanyl or adamantyl.

In some examples, the Rb is

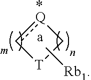

In some examples, the Rb is

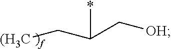

and the f is 1, 2, or 3.

In some examples, the Rb is

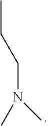

In some examples, the Rb is adamantyl.

In some examples, the Rb is 3-oxaspiro[3,3]heptanyl.

In some examples, the Rc is

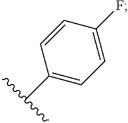 [SMILES: *c1ccc(F)cc1]

the Ra is a substituted or unsubstituted phenyl, or a substituted or unsubstituted

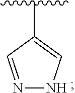 [SMILES: *c1cn[nH]c1]

the substituents are each independently selected from F, methyl, and methoxy; and the number of the respective substituents of the phenyl or the

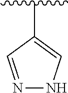 [SMILES: *c1cn[nH]c1]

is independently 0 or 1; the R₁a is selected from H,

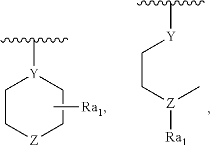

or methyl; the Y is selected from N and O; and the Z is N or O;
the Ra₁ is H, methyl, or ethyl; the A is N, wherein N is substituted by one H; the t is 0; and the Rb is selected from groups in one or more groups of the following 1)-4):
1) a substituted or unsubstituted 7-membered spiroheterocycle, wherein a heteroatom in the spiroheterocycle is O;
2) a substituted or unsubstituted adamantyl, wherein the substituent of the adamantyl is one or more OHs;
3) a substituted or unsubstituted amino, wherein the substituent of the amino is one or two C₁₋₃ alkyls; and
4)

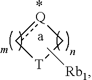

wherein the number of the double bonds a is 0 or 1; the position of the a is any position with a reasonable valence bond in the ring; the m is 0, 1, 2, or 3; the n is 0, 1, or 2; the Q is C; the T is N, O or C; and the insufficient valence bonds are complemented by H;
the number of the Rb₁ is 0, 1, or 2; the Rb₁ is amino, hydroxy, or hydroxymethyl; and the substituent of the C₁₋₃ alkyl is halogen or hydroxyl; and
the R₁b is H, amino, hydroxy, or hydroxymethyl.

In some examples, the Rc is

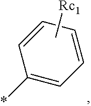

and the Rc₁ is F and located at the para-position on the benzene ring; the Ra is a substituted or unsubstituted phenyl, or a substituted or unsubstituted

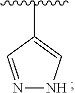 [SMILES: *c1cn[nH]c1]

the substituents are each independently C₁₋₃ alkyl; the number of the respective substituents of the phenyl or the

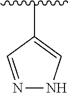 [SMILES: *c1cn[nH]c1]

is 0 or 1; the R₁a is selected from H,

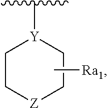

or C₁₋₃ alkyl; the Y is C or N; the Z is N; the Ra₁ is H, CH₃, and CH₂CH₃; the A is N and the N is substituted by one H; the t is selected from 0; the Rb is

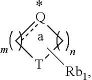

wherein the Q and the T are both C; the a is 0 and the insufficient valence bonds are complemented by H; the m is 2; and the n is 2; and
the number of the Rb₁ is 0, 1, or 2; the Rb₁ is NH₂, hydroxymethyl, or hydroxyethyl; and the R₁b is H, NH₂, or HOCH₂.

In some examples, the Rb is selected from groups in one or more groups of the following 1)-4):
1) a substituted or unsubstituted 7-membered spiroheterocycle, wherein a heteroatom in the spiroheterocycle is O;
2) a substituted or unsubstituted adamantyl, wherein the substituent of the adamantyl is one or more OHs;
3) a substituted or unsubstituted amino, wherein the substituent of the amino is one or two C1-3 alkyls; and
4)

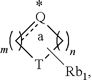

wherein the number of the double bonds a is 0 or 1; the position of the a is any position with a reasonable valence bond in the ring; the m is 0, 1, 2, or 3; the n is 0, 1, or 2; the Q is C; the T is N, O or C; and the insufficient valence bonds are complemented by H.

As shown in formula I, the Rc is a substituted or unsubstituted phenyl, and as shown in formula II

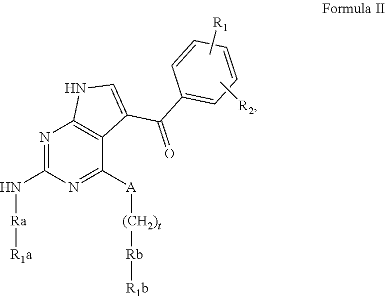

the R₁ and the R₂ are each independently selected from at least one of H, halogen, and C1-3 alkyl.

In some examples, the R₁ and the R₂ are each independently selected from at least one of H, F, Cl, Br, methyl, and ethyl.

In some examples, the R₁ and the R₂ are each independently selected from at least one of H, F, Cl, and methyl.

As shown in formula I, the Rc is a substituted or unsubstituted phenyl, the Rₐ is selected from a substituted or unsubstituted benzene ring, and the Rb is selected from

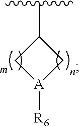

the R₁b is H and as shown in formula III F

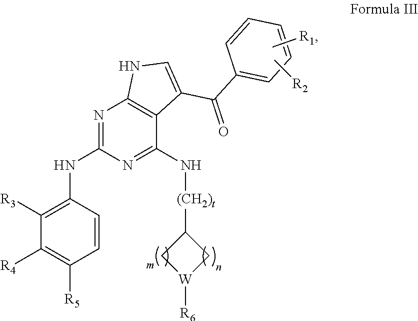

wherein the m is selected from 0, 1, or 2; the n is selected from 0, 1, or 2; the t is selected from 0, 1, and 2; and the W is selected from C, O, or N;
the R₁ is selected from H, F, and Cl;
the R₂ is selected from H, F, and Cl;
the R₃ is selected from H and C₁₋₃ alkoxy;
the R₄ is selected from H and F; and
the R₅ is selected from the range defined above for the R₁a.

In some examples, the R₅ is selected from

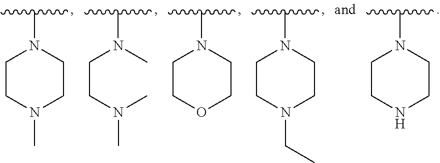

In some examples, the R₅ is

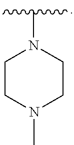 [SMILES: *N1CCN(C)CC1]

In some examples, the R₆ is selected from H, NH₂, hydroxyl, C₁₋₃ alkoxy, C₁₋₃ alkyl substituted by hydroxyl, SO₂R₂b, C₁₋₃ alkyl,

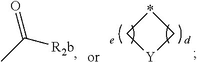

each R₂b is independently a substituted or unsubstituted C₁₋₃ alkyl, a substituted or unsubstituted phenyl, C₃₋₆ cycloalkyl, C₂₋₅ alkenyl, or

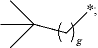

the substituent of the C₁₋₃ alkyl or the phenyl is halogen or hydroxyl; the g is 1, 2, or 3; the e and the d are each independently selected from 0, 1, 2, or 3; and the Y is selected from C, O, or N.

In some examples, the R₃ is selected from H, methoxy, and ethoxy.

In some examples, the R₆ is H, NH₂, HOCH₂, OH, SO₂R₂b, CH₃, CH₃CH₂, (CH₃)₂CH₂,

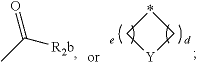

each R₂b is independently methyl, ethyl, propyl, isopropyl, hydroxymethyl, hydroxyethyl, hydroxypropyl, cyclopropyl, cyclobutyl, cyclopentyl, cyclooctyl, ethenyl, propenyl, or

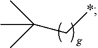

and the g is 1, 2, or 3.

In some examples, the R₆ is H, amino, hydroxy, C₁₋₃ alkyl substituted by hydroxy, C₁₋₃ alkoxy,

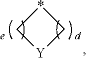

or SO₂R₂b, and each R₂b is independently selected from C₁₋₃ alkyl or C₃₋₆ cycloalkyl; and the e and the d are each independently selected from 1, 2, or 3; and the Y is selected from C, N, or O;

In some examples, the R₆ is H, amino, hydroxy, C₁₋₃ alkyl, C₁₋₃ alkyl substituted by hydroxy, C₁₋₃ alkoxy, or SO₂R₂b and each R₂b is independently selected from C₁₋₃ alkyl.

In some examples, the R₆ is H, amino, hydroxy, C₁₋₃ alkyl, C₁₋₃ alkoxy, or C₁₋₃ alkyl substituted by hydroxy.

In some examples, the R₆ is H, amino, hydroxy, or C₁₋₃ alkyl substituted by hydroxy.

In some examples, the R₆ is H, amino, or C₁₋₃ alkyl substituted by hydroxy.

In some examples, the R₆ is selected from H, OH, CH₂OH,

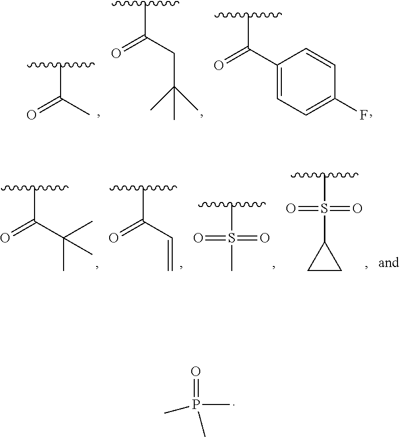

In some examples, the R₆ is CH₂OH.

In some examples, as shown in formula I, the Ra is a substituted or unsubstituted

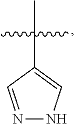 [SMILES: *c1cn[nH]c1]

and the substituent is at least one of H, C₁₋₃ alkoxy, and C₁₋₃ alkyl; and further, the substituent is one, two or three of H, methoxy, ethoxy, propoxy, isopropoxy, methyl, ethyl, and propyl.

In some examples, as shown in formula I, the Ra is

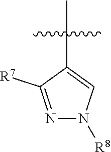

and the Rb is selected from

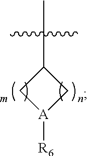

the R₁b H and as shown in formula IV

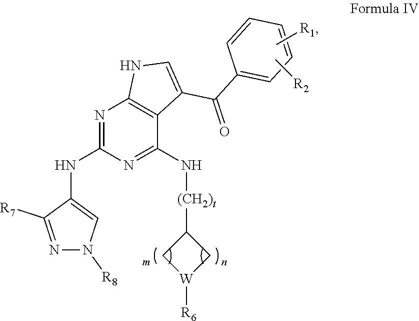

wherein the m is selected from 0, 1, or 2; the n is selected from 0, 1, or 2; the t is selected from 0, 1, and 2; and the W is C, O, or N;
the R₁ is selected from H, F, and Cl;
the R₂ is selected from H, F, and Cl;
the R₇ is selected from H or C₁₋₃ alkoxy; the R₆ is selected from H, OH, CH₂

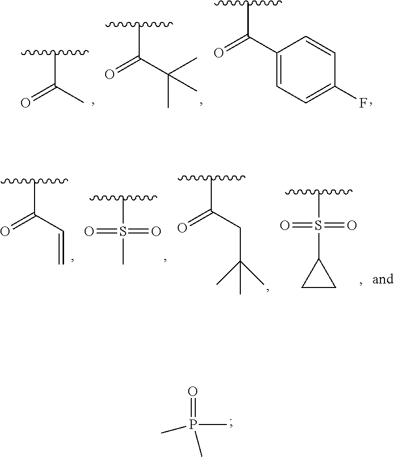

and
the R₈ is selected from C₁₋₃ alkyl or piperidin substituted by C₁₋₃ alkyl.

In some examples, the R₈ is methyl.

In some examples, the R₈ is ethyl.

In some examples, the R₈ is methylpiperidin. In some examples, the Rb is

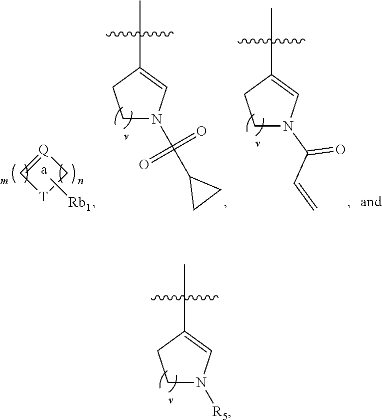

and each v is independently 1, 2, or 3.

In some examples, the substituent of the adamantyl is at the position 2 or 3 of the adamantyl.

### Some Descriptions

In the description of the general formula in the present application, functional groups, substituents, or bonds are emphasized. If there are insufficient valence bonds, not specifically described, are by default complemented by H.

The linking position between the groups, not specifically described, is any position with a reasonable valence bond.

The substituent of the bridge ring is one or more independent amino groups, C₁₋₃ alkyl, C₁₋₃ alkoxy, and OH. It is independently explained that when a plurality of substituents are present, each substituent may be the same or different, such as a plurality of amino or a plurality of C₁₋₃ alkyl, a plurality of C₁₋₃ alkoxy, or a plurality of OHs, and a combination of one amino, one methyl, one ethoxy, one hydroxyl, and one propyl as a bridge ring.

“Each R₂b is independently selected from” only refers that if there are multiple R₂b in the same formula, each R₂b may be the same or different, and independent of each other.

“R₁b is selected from any one of the following groups” refers any one group of the following groups of “R₁b is”. The selection ends before a new paragraph (space before paragraph).

Some new starting line top-written formats herein belong to the intra-paragraph content. The line division is for clarity of form and convenience of reading, and should not be used as a basis for the isolated content similar to a new paragraph.

“R₁b is” means that both may be the same or different groups. When the groups are the same, further substituents may be the same or different, for example

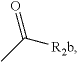

the R₂b in the Rb₁ and the R₂b in the Rib may be the same group or different groups.

In some examples, the compound is selected from the following specific compounds:

(R)-(4-((1-(cyclopropylsulfonyl)piperidin-3-yl)amino)-2-((4-(4-methylpiperazin-1-yl)phenyl)amino)-7H-pyrrolo[2,3-d]pyrimidin-5-yl)(4-fluorophenyl)methanone, (R)-(4-(1-(cyclopropylsulfonyl)piperidin-3-yl)amino)-2-((1-methyl-1H-pyrazol-4-yl)amino)-7H-pyrrolo[2,3-d]pyrimidin-5-yl)(4-fluorophenyl)methanone, (R)-1-(4-((1-(cyclopropylsulfonyl)piperidin-3-yl)amino)-2-((-4-(4-methylpiperazin-1-yl)phenyl)amino)-7H-pyrrolo[2,3-d]pyrimidin-5-yl)-2-methylpropan-1-one, (S)-(4-(1-(cyclopropylsulfonyl)piperidin-3-yl)amino)-2-((4-(4-methylpiperazin-1-yl)phenyl)amino)-7H-pyrrolo[2,3-d]pyrimidin-5-yl)(4-fluorophenyl)methanone, (R)-1-(3-((5-(4-fluorobenzoyl)-2-((4-(4-methylpiperazin-1-yl)phenyl)amino)-7H-pyrrolo[2,3-d]pyrimidin-4-yl)amino)piperidin-1-yl)prop-2-en-1-one, (4-fluorophenyl)(2-((4-(4-methylpiperazin-1-yl)phenyl)amino)-4-((tetrahydro-2H-pyran-4-yl)amino)-7H-pyrrolo[2,3-d]pyrimidin-5-yl)methanone, (4-fluorophenyl)(2-((4-(4-methylpiperazin-1-yl)phenyl)amino)-4-(1-(tetrahydro-2H-pyran-4-yl)piperidin-4-yl)amino)-7H-pyrrolo[2,3-d]pyrimidin-5-yl)methanone, (4-fluorophenyl)(4-(((1r,4r)-4-(hydroxymethyl)cyclohexyl)amino)-2-((2-methoxy-4-morpholinophenyl)amino)-7H-pyrrolo[2,3-d]pyrimidin-5-yl)methanone, (3,4-difluorophenyl)(4-((1r,4r)-4-(hydroxymethyl)cyclohexyl)amino)-2-((4-morpholinophenyl)amino)-7H-pyrrolo[2,3-d]pyrimidin-5-yl)methanone, (3-fluorophenyl)(4-(((1r,4r)-4-(hydroxymethyl)cyclohexyl)amino)-2-((4-morpholinophenyl)amino)-7H-pyrrolo[2,3-d]pyrimidin-5-yl)methanone, (4-fluorophenyl)(4-((1-isopropylpiperidin-4-yl)amino)-2-((4-morpholinophenyl)amino)-7H-pyrrolo[2,3-d]pyrimidin-5-yl)methanone, (3,5-difluorophenyl)(4-(((1r,4r)-4-(hydroxymethyl)cyclohexyl)amino)-2-((4-morpholinophenyl)amino)-7H-pyrrolo[2,3-d]pyrimidin-5-yl)methanone, and (2-chloro-4-fluorophenyl)(4-(((1r,4r)-4-(hydroxymethyl)cyclohexyl)amino)-2-((4-morpholinophenyl)amino)-7H-pyrrolo[2,3-d]pyrimidin-5-yl)methanone.

In some examples, the compound is selected from the following specific compounds: (S)-1-(3-((5-(4-fluorobenzoyl)-2-((4-(4-methylpiperazin-1-yl)phenyl)amino)-7H-pyrrolo[2,3-d]pyrimidin-4-yl)amino)pyrrolidin-1-yl)-2,2-dimethylpropan-1-one, (4-fluorophenyl)(4-(((1r,4r)-4-hydroxycyclohexyl)amino)-2-((4-(4-methylpiperazin-1-yl)phenyl)amino)-7H-pyrrolo[2,3-d]pyrimidin-5-yl)methanone, (4-fluorophenyl)(4-((1-(hydroxymethyl)cyclopropyl)amino)-2-((4-(4-methylpiperazin-1-yl)phenyl)amino)-7H-pyrrolo[2,3-d]pyrimidin-5-yl)methanone, (2-((3-fluoro-4-(4-methylpiperazin-1-yl)phenyl)amino)-4-(((1r,4r)-4-(hydroxymethyl)cyclohexyl)amino)-7H-pyrrolo[2,3-d]pyrimidin-5-yl)(4-fluorophenyl)methanone, (4-fluorophenyl)(4-((3R,6S)-6-(hydroxymethyl)tetrahydro-2H-pyran-3-yl)amino)-2-((4-morpholinophenyl)amino)-7H-pyrrolo[2,3-d]pyrimidin-5-yl)methanone, (4-fluorophenyl)(4-((3R,6S)-6-(hydroxymethyl)tetrahydro-2H-pyran-3-yl)amino)-2-((4-(4-methylpiperazin-1-yl)phenyl)amino)-7H-pyrrolo[2,3-d]pyrimidin-5-yl)methanone, (2,4-difluorophenyl)(4-(((1r,4r)-4-(hydroxymethyl)cyclohexyl)amino)-2-((4-morpholinophenyl)amino)-7H-pyrrolo[2,3-d]pyrimidin-5-yl)methanone, (4-((2-oxaspiro[3.3]hept-6-yl)amino)-2-((4(4-methylpiperazin-1-yl)phenyl)amino)-7H-pyrrolo[2,3-d]pyrimidin-5-yl)(4-fluorophenyl)methanone, (4-fluorophenyl)(4-(((1s,4s)-4-(hydroxymethyl)cyclohexyl)amino)-2-((4-morpholinophenyl)amino)-7H-pyrrolo[2,3-d]pyrimidin-5-yl)methanone, (2-((3-fluoro-4-(4-methylpiperazin-1-yl)phenyl)amino)-4-(((1s,4s)-4-(hydroxymethyl)cyclohexyl)amino)-7H-pyrrolo[2,3-d]pyrimidin-5-yl)(4-fluorophenyl)methanone, (4-fluorophenyl)(4-(((1s,4s)-4-(hydroxymethyl)cyclohexyl)amino)-2-((2-methoxy-4-(4-methylpiperazin-1-yl)phenyl)amino)-7H-pyrrolo[2,3-d]pyrimidin-5-yl)methanone, (2-((4-(2-(dimethylamino)ethoxy)phenyl)amino)-4-(((1s,4s)-4-(hydroxymethyl)cyclohexyl)amino)-7H-pyrrolo[2,3-d]pyrimidin-5-yl)(4-fluorophenyl)methanone, (4-fluorophenyl)(4-(((1s,4s)-4-(hydroxymethyl)cyclohexyl)amino)-2-((3-methoxy-1-methyl-1H-pyrazol-4-yl)amino)-7H-pyrrolo[2,3-d]pyrimidin-5-yl)methanone, (4-fluorophenyl)(4-((1S,4R)-4-(hydroxymethyl)cyclopent-2-en-1-yl)amino)-2-((4-(4-methylpiperazin-1-yl)phenyl)amino)-7H-pyrrolo[2,3-d]pyrimidin-5-yl)methanone, (4-fluorophenyl)(4-((1R,4S)-4-(hydroxymethyl)cyclopent-2-en-1-yl)amino)-2-((4-(4-methylpiperazin-1-yl)phenyl)amino)-7H-pyrrolo[2,3-d]pyrimidin-5-yl)methanone, (4-fluorophenyl)(4-(((1r,3s,5R,7S)-3-hydroxyadamantan-1-yl)amino)-2-((4-(4-methylpiperazin-1-yl)phenyl)amino)-7H-pyrrolo[2,3-d]pyrimidin-5-yl)methanone, (4-fluorophenyl)(4-(((1s,3R,4s,5S,7s)-4-hydroxyadamantan-1-yl)amino)-2-((4-(4-methylpiperazin-1-yl)phenyl)amino)-7H-pyrrolo[2,3-d]pyrimidin-5-yl)methanone, and (4-fluorophenyl)(4-(((1r,4r)-4-(hydroxymethyl)cyclohexyl)amino)-2-((1-methyl-1H-pyrazol-4-yl)amino)-7H-pyrrolo[2,3-d]pyrimidin-5-yl)methanone.

In some examples, the compound is selected from the following specific compounds: (4-fluorophenyl)(4-(((1r,4r)-4-(hydroxymethyl)cyclohexyl)amino)-2-((4-(4-methylpiperazin-1-yl)phenyl)amino)-7H-pyrrolo[2,3-d]pyrimidin-5-yl)methanone, (4-fluorophenyl)(2-((4-(4-methylpiperazin-1-yl)phenyl)amino)-4-((tetrahydro-2H-pyran-4-yl)methyl)amino)-7H-pyrrolo[2,3-d]pyrimidin-5-yl)methanone, (4-((2-(dimethylamino)ethyl)amino)-2-((4-(4-methylpiperazin-1-yl)phenyl)amino)-7H-pyrrolo[2,3-d]pyrimidin-5-yl)(4-fluorophenyl)methanone, (4-fluorophenyl)(4-(((1r,4r)-4-(hydroxymethyl)cyclohexyl)amino)-2-(4-morpholinophenyl)amino)-7H-pyrrolo[2,3-d]pyrimidin-5-yl)methanone, and (4-(((1r,4r)-4-(hydroxymethyl)cyclohexyl)amino)-2-(4-morpholinophenyl)amino)-7H-pyrrolo[2,3-d]pyrimidin-5-yl)(p-tolyl)methanone.

In some examples, the compound is selected from the following specific compounds: (4-(cyclohexylamino)-2-((4-(4-methylpiperazin-1-yl)phenyl)amino)-7H-pyrrolo[2,3-d]pyrimidin-5-yl)(4-fluorophenyl)methanone, (4-fluorophenyl)(4-((2-hydroxycyclohexyl)amino)-2-((4-(4-methylpiperazin-1-yl)phenyl)amino)-7H-pyrrolo[2,3-d]pyrimidin-5-yl)methanone, (R)-(4-fluorophenyl)(2-((4-(4-methylpiperazin-1-yl)phenyl)amino)-4-(1-(methylsulfonyl)pyrrolidin-3-yl)amino)-7H-pyrrolo[2,3-d]pyrimidin-5-yl)methanone, (R)-(4-fluorophenyl)(2-((4-(4-methylpiperazin-1-yl)phenyl)amino)-4-(1-(methylsulfonyl)piperidin-3-yl)amino)-7H-pyrrolo[2,3-d]pyrimidin-5-yl)methanone, (S)-(4-fluorophenyl)(2-((4-(4-methylpiperazin-1-yl)phenyl)amino)-4-(tetrahydro-2H-pyran-3-yl)amino)-7H-pyrrolo[2,3-d]pyrimidin-5-yl)methanone, (R)-(4-fluorophenyl)(2-((4-(4-methylpiperazin-1-yl)phenyl)amino)-4-(tetrahydro-2H-pyran-3-yl)amino)-7H-pyrrolo[2,3-d]pyrimidin-5-yl)methanone, (4-fluorophenyl)(2-((4-(4-methylpiperazin-1-yl)phenyl)amino)-4-((tetrahydro-2H-pyran-4-yl)methyl)amino)-7H-pyrrolo[2,3-d]pyrimidin-5-yl)methanone, (4-((1-(cyclopropylsulfonyl)piperidin-4-yl)amino)-2-((4-(4-methylpiperazin-1-yl)phenyl)amino)-7H-pyrrolo[2,3-d]pyrimidin-5-yl)(4-fluorophenyl)methanone, (4-fluorophenyl)(2-((4-(4-methylpiperazin-1-yl)phenyl)amino)-4-((1-(methylsulfonyl)piperidin-4-yl)amino)-7H-pyrrolo[2,3-d]pyrimidin-5-yl)methanone, (S)-(4-fluorophenyl)(2-((4-(4-methylpiperazin-1-yl)phenyl)amino)-4-(tetrahydrofuran-3-yl)amino)-7H-pyrrolo[2,3-d]pyrimidin-5-yl)methanone, (R)-(4-fluorophenyl)(2-((4-(4-methylpiperazin-1-yl)phenyl)amino)-4-(tetrahydrofuran-3-yl)amino)-7H-pyrrolo[2,3-d]pyrimidin-5-yl)methanone, (4-fluorophenyl)(4-((1-(hydroxymethyl)cyclopentyl)amino)-2-((4-(4-methylpiperazin-1-yl)phenyl)amino)-7H-pyrrolo[2,3-d]pyrimidin-5-yl)methanone, (4-fluorophenyl)(4-((1r,4r)-4-methoxycyclohexyl)amino)-2-((4-(4-methylpiperazin-1-yl)phenyl)amino)-7H-pyrrolo[2,3-d]pyrimidin-5-yl)methanone, (4-fluorophenyl)(4-(((1r,4r)-4-(hydroxymethyl)cyclohexyl)amino)-2-((2-methoxy-4-(4-methylpiperazin-1-yl)phenyl)amino)-7H-pyrrolo[2,3-d]pyrimidin-5-yl)methanone, (2-((3-fluoro-4-morpholinophenyl)amino)-4-(((1r,4r)-4-(hydroxymethyl)cyclohexyl)amino)-7H-pyrrolo[2,3-d]pyrimidin-5-yl)(4-fluorophenyl)methanone, (4-fluorophenyl)(2-((4-(4-methylpiperazin-1-yl)phenyl)amino)-4-((tetrahydro-2H-pyran-4-yl)oxy)-7H-pyrrolo[2,3-d]pyrimidin-5-yl)methanone, (4-fluorophenyl)(2-((4-(4-methylpiperazin-1-yl)phenyl)amino)-4-((1-methylpiperidin-4-yl)amino)-7H-pyrrolo[2,3-d]pyrimidin-5-yl)methanone, (4-fluorophenyl)(4-(((1r,4r)-4-(hydroxymethyl)cyclohexyl)amino)-2-((3-methoxy-1-methyl-1H-pyrazol-4-yl)amino)-7H-pyrrolo[2,3-d]pyrimidin-5-yl)methanone, ((1r,4r)-4-((2-((1-methyl-1H-pyrazol-4-yl)amino)-7H-pyrrolo[2,3-d]pyrimidin-4-yl)amino)cyclohexyl)methanol, (4-(((1r,4r)-4-(hydroxymethyl)cyclohexyl)amino)-2-((4-morpholinophenyl)amino)-7H-pyrrolo[2,3-d]pyrimidin-5-yl)(phenyl)methanone, and (4-(((1r,4r)-4-(hydroxymethyl)cyclohexyl)amino)-2-((4-morpholinophenyl)amino)-7H-pyrrolo[2,3-d]pyrimidin-5-yl)(2,3,4-trifluorophenyl)methanone.

In some examples, the compound is selected from the following specific compounds:

(S)-(4-(1-(cyclopropylsulfonyl)piperidin-3-yl)amino)-2-((4-morpholinophenyl)amino)-7H-pyrrolo[2,3-d]pyrimidin-5-yl(4-fluorophenyl)methanone, ((1r,4r)-4-((2-((4-morpholinophenyl)amino)-7H-pyrrolo[2,3-d]pyrimidin-4-yl)amino)cyclohexyl)methanol, (4-fluorophenyl)(4-((1-isopropylpiperidin-4-yl)amino)-2-((4-(4-methylpiperazin-1-yl)phenyl)amino)-7H-pyrrolo[2,3-d]pyrimidin-5-yl)methanone, (4-fluorophenyl)(4-((1-methylpiperidin-4-yl)amino)-2-((4-morpholinophenyl)amino)-7H-pyrrolo[2,3-d]pyrimidin-5-yl)methanone.

In some examples, the compound is selected from the following specific compounds: (4-(((1s,4s)-4-aminocyclohexyl)amino)-2-((4-(4-methylpiperazin-1-yl)phenyl)amino)-7H-pyrrolo[2,3-d]pyrimidin-5-yl)(4-fluorophenyl)methanone, (2-((4-(4-ethylpiperazin-1-yl)phenyl)amino)-4-(((1s,4s)-4-(hydroxymethyl)cyclohexyl)amino)-7H-pyrrolo[2,3-d]pyrimidin-5-yl)(4-fluorophenyl)methanone, (4-fluorophenyl)(4-(((1s,4s)-4-(hydroxymethyl)cyclohexyl)amino)-2-((1-methyl-1H-pyrazol-4-yl)amino)-7H-pyrrolo[2,3-d]pyrimidin-5-yl)methanone, (4-fluorophenyl)(4-(((1s,4s)-4-(hydroxymethyl)cyclohexyl)amino)-2-((1-(1-methylpiperidin-4-yl)-1H-pyrazol-4-yl)amino)-7H-pyrrolo[2,3-d]pyrimidin-5-yl)methanone, and (4-fluorophenyl)(4-(((1s,4s)-4-(hydroxymethyl)cyclohexyl)amino)-2-((4-(4-methylpiperazin-1-yl)phenyl)amino)-7H-pyrrolo[2,3-d]pyrimidin-5-yl)methanone.

In some examples, the compound is selected from the following specific compounds: (R)-(4-(1-(cyclopropylsulfonyl)pyrrolidin-3-yl)amino)-2-((4-(4-methylpiperazin-1-yl)phenyl)amino)-7H-pyrrolo[2,3-d]pyrimidin-5-yl)(4-fluorophenyl)methanone, (S)-(4-(1-(cyclopropylsulfonyl)pyrrolidin-3-yl)amino)-2-((4-(4-methylpiperazin-1-yl)phenyl)amino)-7H-pyrrolo[2,3-d]pyrimidin-5-yl)(4-fluorophenyl)methanone, (R)-1-(3-((5-(4-fluorobenzoyl)-2-((4-(4-methylbenzoyl-1-yl)phenyl)amino)-7H-pyrrolo[2,3-d]pyrimidin-4-yl)amino)piperidin-1-yl)prop-2-en-1-one, (S)-1-(3-((5-(4-fluorobenzoyl)-2-((4-(4-methylpiperazin-1-yl)phenyl)amino)-7H-pyrrolo[2,3-d]pyrimidin-4-yl)amino)piperidin-1-yl)prop-2-en-1-one, (S)-1-(3-((5-(4-fluorobenzoyl)-2-((4-(4-methylpiperazin-1-yl)phenyl)amino)-7H-pyrrolo[2,3-d]pyrimidin-4-yl)amino)pyrrolidin-1-yl)prop-2-en-1-one, (S)-(4-(1-(cyclopropylsulfonyl)piperidin-3-yl)amino)-2-((3-fluoro-4-(4-methylpiperazin-1-yl)phenyl)amino)-7H-pyrrolo[2,3-d]pyrimidin-5-yl)(4-fluorophenyl)methanone, (S)-(4-(1-(cyclopropylsulfonyl)piperidin-3-yl)amino)-2-((4-(4-ethylpiperazin-1-yl)phenyl)amino)-7H-pyrrolo[2,3-d]pyrimidin-5-yl)(4-fluorophenyl)methanone, (S)-(4-(1-(cyclopropylsulfonyl)pyrrolidin-3-yl)amino)-2-((4-morpholinophenyl)amino)-7H-pyrrolo[2,3-d]pyrimidin-5-yl)(4-fluorophenyl)methanone, (S)-(4-(1-(cyclopropylsulfonyl)pyrrolidin-3-yl)amino)-2-((4-(4-methylpiperazin-1-phenyl)amino)-7H-pyrrolo[2,3-d]pyrimidin-5-yl)(phenyl)methanone, (S)-1-(3-((5-benzoyl-2-((4-(4-methylpiperazin-1-yl)phenyl)amino)-7H-pyrrolo[2,3-d]pyrimidin-4-yl)amino)pyrrolidin-1-yl)prop-2-en-1-one, (S)-(4-(1-(cyclopropylsulfonyl)pyrrolidin-3-yl)amino)-2-((4-(4-methylpiperazin-1-yl)phenyl)amino)-7H-pyrrolo[2,3-d]pyrimidin-5-yl)(3-fluorophenyl)methanone, (S)-1-(3-((5-(3-fluorobenzoyl)-2-((4-(4-methylpiperazin-1-yl)phenyl)amino)-7H-pyrrolo[2,3-d]pyrimidin-4-yl)amino)pyrrolidin-1-yl)prop-2-en-1-one, (S)-(4-(1-(cyclopropylsulfonyl)pyrrolidin-3-yl)amino)-2-((4-(4-methylpiperazin-1-yl)phenyl)amino)-7H-pyrrolo[2,3-d]pyrimidin-5-yl)(3,4-difluorophenyl)methanone, (S)-1-(3-((5-(3,4-difluorobenzoyl)-2-((4-(4-methylpiperazin-1-yl)phenyl)amino)-7H-pyrrolo[2,3-d]pyrimidin-4-yl)amino)pyrrolidin-1-yl)prop-2-en-1-one, (4-fluorophenyl)(2-((4-(4-methylpiperazin-1-yl)phenyl)amino)-4-(morpholinoamino)-7H-pyrrolo[2,3-d]pyrimidin-5-yl)methanone, (4-fluorophenyl)(2-((4-(4-methylpiperazin-1-yl)phenyl)amino)-4-(piperidin-1-ylamino)-7H-pyrrolo[2,3-d]pyrimidin-5-yl)methanone, (S)-1-(3-((5-(2,4-difluorobenzoyl)-2-((4-(4-methylpiperazin-1-yl)phenyl)amino)-7H-pyrrolo[2,3-d]pyrimidin-4-yl)amino)pyrrolidin-1-yl)prop-2-en-1-one, (S)-(4-((1-(cyclopropylsulfonyl)pyrrolidin-3-yl)amino)-2-((4-(4-methylpiperazin-1-yl)phenyl)amino)-7H-pyrrolo[2,3-d]pyrimidin-5-yl)(2-fluorophenyl)methanone, (S)-1-(3-((5-(2-fluorobenzoyl)-2-((4-(4-methylpiperazin-1-yl)phenyl)amino)-7H-pyrrolo[2,3-d]pyrimidin-4-yl)amino)pyrrolidin-1-yl)prop-2-en-1-one, (R)-1-(3-((5-(4-fluorobenzoyl)-2-((1-methyl-1H-pyrazol-4-yl)amino)-7H-pyrrolo[2,3-d]pyrimidin-4-yl)amino)pyrrolidin-1-yl)prop-2-en-1-one, (4-((2-(dimethylphosphoryl)phenyl)amino)-2-((4-(4-methylpiperazin-1-yl)phenyl)amino)-7H-pyrrolo[2,3-d]pyrimidin-5-yl)(4-fluorophenyl)methanone, (S)-(4-((1-(cyclopropylsulfonyl)pyrrolidin-3-yl)amino)-2-((4-(4-methylpiperazin-1-yl)phenyl)amino)-7H-pyrrolo[2,3-d]pyrimidin-5-yl)(2,4-difluorophenyl)methanone, (S)-1-(3-((5-(4-fluorobenzoyl)-2-((4-(4-methylpiperazin-1-yl)phenyl)amino)-7H-pyrrolo[2,3-d]pyrimidin-4-yl)amino)pyrrolidin-1-yl)-3,3-dimethylbut-1-one, (S)-(3-((5-(4-fluorobenzoyl)-2-(4-(4-methylpiperazin-1-yl)phenyl)amino)-7H-pyrrolo[2,3-d]pyrimidin-4-yl)amino)pyrrolidin-1-yl)(4-fluorophenyl)methanone, (S)-1-(3-((5-(4-fluorobenzoyl)-2-((4-(4-methylpiperazin-1-yl)phenyl)amino)-7H-pyrrolo[2,3-d]pyrimidin-4-yl)amino)pyrrolidin-1-yl)-2,2-dimethylprop-1-one, (S)-1-(3-((5-(4-fluorobenzoyl)-2-((4-(4-methylpiperazin-1-yl)phenyl)amino)-7H-pyrrolo[2,3-d]pyrimidin-4-yl)amino)pyrrolidin-1-yl)ethyl-1-one, (4-fluorophenyl)(4-(methyl(tetrahydro-2H-pyran-4-yl)amino)-2-(4-((4-methylpiperazin-1-yl)phenyl)amino)-7H-pyrrolo[2,3-d]pyrimidin-5-yl)methanone, 1-(4-((5-(4-fluorobenzoyl)-2-((4-(4-methylpiperazin-1-yl)phenyl)amino)-7H-pyrrolo[2,3-d]pyrimidin-4-yl)amino)piperidin-1-yl)prop-2-en-1-one, 1-(4-((5-(4-fluorobenzoyl)-2-((4-(4-methylpiperazin-1-yl)phenyl)amino)-7H-pyrrolo[2,3-d]pyrimidin-4-yl)amino)piperidin-1-yl)-2, 2-dimethylprop-1-one, 1-(4-((5-(4-fluorobenzoyl)-2-((4-(4-methylpiperazin-1-yl)phenyl)amino)-7H-pyrrolo[2,3-d]pyrimidin-4-yl)amino)piperidin-1-yl)-3,3-dimethylbut-1-one, (4-fluorophenyl)(2-((4-(4-methylpiperazin-1-yl)phenyl)amino)-4-(oxetan-3-ylamino)-7H-pyrrolo[2,3-d]pyrimidin-5-yl)methanone, (4-fluorophenyl)(4-((3-methyloxetan-3-yl)amino)-2-((4-(4-methylpiperazin-1-yl)phenyl)amino)-7H-pyrrolo[2,3-d]pyrimidin-5-yl)methanone, (S)-(4-fluorophenyl)(4-((1-hydroxy-4-methylpentan-2-yl)amino)-2-((4-(4-methylpiperazin-1-yl)phenyl)amino)-7H-pyrrolo[2,3-d]pyrimidin-5-yl)methanone, (R)-(4-fluorophenyl)(4-((1-hydroxy-4-methylpentan-2-yl)amino)-2-((4-(4-methylpiperazin-1-yl)phenyl)amino)-7H-pyrrolo[2,3-d]pyrimidin-5-yl)methanone, (R)-(4-fluorophenyl)(4-(1-hydroxy-3,3-dimethylbut-2-yl)amino)-2-((4-(4-methylpiperazin-1-yl)phenyl)amino)-7H-pyrrolo[2,3-d]pyrimidin-5-yl)methanone, (S)-(4-fluorophenyl)(4-((1-hydroxy-3,3-dimethylbut-2-yl)amino)-2-((4-(4-methylpiperazin-1-yl)phenyl)amino)-7H-pyrrolo[2,3-d]pyrimidin-5-yl)methanone, (4-fluorophenyl)(2-((4-(4-methylpiperazin-1-yl)phenyl)amino)-7H-pyrrolo[2,3-d]pyrimidin-5-yl)methanone, (4-fluorophenyl)(2-((4-(4-methylpiperazin-1-yl)phenyl)amino)-4-((tetrahydro-2H-pyran-3-yl)amino)-7H-pyrrolo[2,3-d]pyrimidin-5-yl)methanone, (4-fluorophenyl)(2-((4-(4-methylpiperazin-1-yl)phenyl)amino)-4-((2-morpholinoethyl)amino)-7H-pyrrolo[2,3-d]pyrimidin-5-yl)methanone, (4-((2-(diethylamino)ethyl)amino)-2-((4-(4-methylpiperazin-1-yl)phenyl)amino)-7H-pyrrolo[2,3-d]pyrimidin-5-yl)(4-fluorophenyl)methanone, (4-fluorophenyl)(2-((4-(4-methylpiperazin-1-yl)phenyl)amino)-4-((2-(pyrrolidin-1-yl)ethyl)amino)-7H-pyrrolo[2,3-d]pyrimidin-5-yl)methanone, (4-fluorophenyl)(2-((4-(4-methylpiperazin-1-yl)phenyl)amino)-4-((2-(piperidin-1-yl)ethyl)amino)-7H-pyrrolo[2,3-d]pyrimidin-5-yl)methanone, (4-fluorophenyl)(4-((4-hydroxytetrahydro-2H-pyran-4-yl)methyl)amino)-2-((4-(4-methylpiperazin-1-yl)phenyl)amino)-7H-pyrrolo[2,3-d]pyrimidin-5-yl)methanone, (4-fluorophenyl)(4-((3-(hydroxymethyl)phenyl)amino)-2-((4-(4-methylpiperazin-1-yl)phenyl)amino)-7H-pyrrolo[2,3-d]pyrimidin-5-yl)methanone, (4-fluorophenyl)(4-((6-(hydroxymethyl)pyridin-3-yl)amino)-2-((4-(4-methylpiperazin-1-yl)phenyl)amino)-7H-pyrrolo[2,3-d]pyrimidin-5-yl)methanone, (4-(((1r,4r)-4-aminocyclohexyl)amino)-2-(4-(4-methylpiperazin-1-yl)phenyl)amino)-7H-pyrrolo[2,3-d]pyrimidin-5-yl)(4-fluorophenyl)methanone, (4-fluorophenyl)(4-(((1r,4r)-4-(hydroxymethyl)cyclohexyl)amino)-2-((1-(1-methylpiperidin-4-yl)-1H-pyrazol-4-yl)amino)-7H-pyrrolo[2,3-d]pyrimidin-5-yl)methanone, (4-((2-((4-(4-methylpiperazin-1-yl)phenyl)amino)-7H-pyrrolo[2,3-d]pyrimidin-4-yl)amino)piperidin-1-yl)methanol, (4-fluorophenyl)(4-(((1r,4r)-4-(hydroxymethyl)cyclohexyl)amino)-2-((3-methoxy-1-(1-methylpiperidin-4-yl)-1H-pyrazol-4-yl)amino)-7H-pyrrolo[2,3-d]pyrimidin-5-yl)methanone, and ((1r,4r)-4-((2-((4-morpholinophenyl)amino)thieno[2,3-d]pyrimidin-4-yl)amino)cyclohexyl)methanol.

The present invention provides a method for preparing a compound of formula I, including an isomer thereof, a hydrate thereof, a solvate thereof, a pharmaceutically acceptable salt thereof, and a prodrug thereof, comprising the following steps:

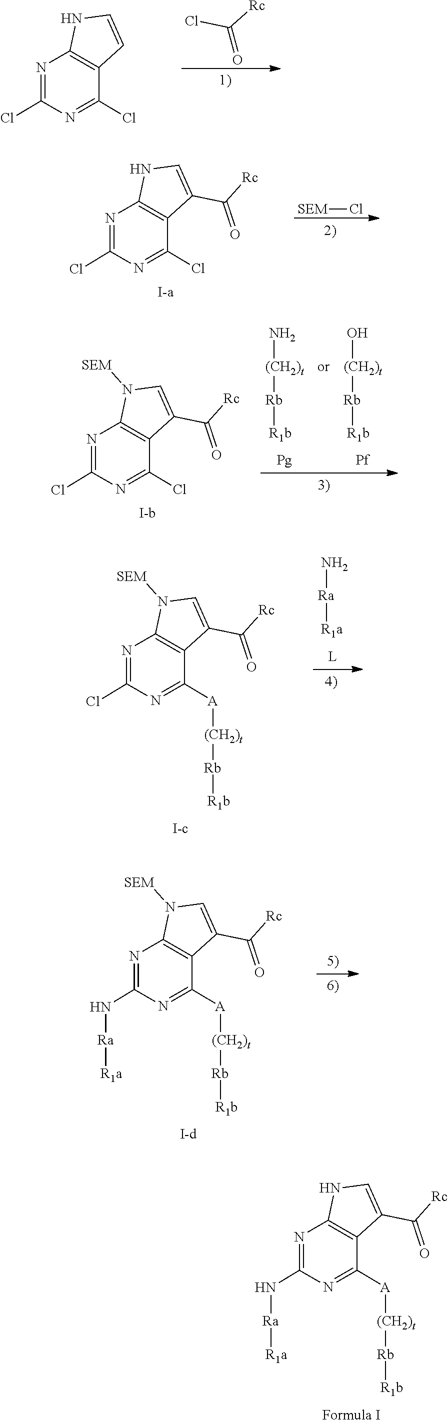

1) reacting 2,4-dichloro-7H-pyrrolo[2,3-d]pyrimidine with

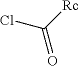

to obtain a compound of formula I-a, preferably, performing the reaction in an organic solvent, which is a polar solvent, preferably, dichloromethane or acetone;
2) reacting the compound I-a with 2-(trimethylsilyl)ethoxymethyl chloride (SEM-Cl) under the heating condition to obtain the compound I-b, preferably, at 30-60° C.;
performing the reaction in an organic solvent: at least one of N,N-dimethylformamide (DMF), N,N-dimethylacetamide (DMA), N,N-diisopropylethylamine (DIPEA), N-methyl-2-pyrrolidone (NMP), ethylene glycol dimethyl ether, isopropanol, n-butanol, sec-butanol, and tert-butanol; and
performing the reaction under the alkaline condition, wherein the alkali is an inorganic alkali or an organic alkali, the inorganic alkali is selected from ammonia, potassium carbonate, sodium carbonate, and cesium carbonate, and the organic alkali is selected from at least one of sodium acetate, triethylamine, diisopropylethylamine, triethylene diamine, pyridine, 4-dimethylamino pyridine, 1,8-diazabicyclo undec-7-ene, and N-methylmorpholine;
3) reacting the compound I-b with the amine Pg and the alcohol Pf under the heating condition to obtain the compound I-c,
preferably, at 80-100° C.; and
performing the reaction in an organic solvent and under the alkaline condition, wherein the alkali and the organic solvent are independently selected from the relevant limitations in step 2);
4) reacting the compound I-c with the amine L under the heating condition to obtain the compound I-d, preferably, at 100-130° C.;
performing the reaction in an organic solvent and under the alkaline condition, wherein the alkali and the organic solvent are independently selected from the relevant limitations in step 2,
wherein the reaction is Buchwald-hartwig coupling using a palladium catalyst and an organic phosphine ligand, the used palladium catalyst is selected from palladium acetate, palladium dichloride, [1,1′-bis(diphenylphosphino)ferrocene]palladium dichloride, tris(dibenzylideneacetone)dipalladium, tetratriphenylphosphine palladium, and bistriphenylphosphine palladium dichloride, and the used organic phosphine ligand includes but is not limited to 2-dicyclohexylphosphine-2′,4′,6′-triisopropylbiphenyl, 1,1′-binaphthyl-2,2′-bisdiphenylphosphine, and 2-dicyclohexylphosphine-2′,6′-diisopropoxy-1,1′-biphenyl;
5) removing trimethylsilyl ethoxymethyl (an SEM group) from the compound I-d under the acid condition, wherein the acid is an inorganic acid or an organic acid, the inorganic acid is selected from at least one of hydrochloric acid, hydrobromic acid, hydrofluoric acid, hydroiodic acid, nitric acid, nitrous acid, and metaphosphoric acid, and the organic acid is selected from at least one of acetic acid, trichloroacetic acid, trifluoroacetic acid, glycolic acid, sulfamic acid, p-toluenesulfonic acid, ethylenediaminetetraacetic acid (EDTA), oxalic acid, and citric acid; and
6) neutralizing the product obtained in step 5 under the alkali condition to obtain the compound of formula I,
wherein the alkali is an inorganic alkali or an organic alkali, the inorganic alkali is selected from ammonia, potassium carbonate, sodium carbonate, and cesium carbonate, and the organic alkali is selected from at least one of sodium acetate, triethylamine, diisopropylethylamine, triethylene diamine, pyridine, 4-dimethylamino pyridine, 1,8-diazabicyclo undec-7-ene, and N-methylmorpholine; and
the Rc, the Ra, the Rb, the R₁a, and the R₁b are as defined above, and the E is a protecting group selected from the SEM group, benzyloxycarbonyl, p-toluenesulfonyl, benzenesulfonyl, acetyl, trifluoroacetyl, fluorenylmethoxycarbonyl, and benzyl.

The present invention provides a method for preparing a compound of formula II, including an isomer thereof, a hydrate thereof, a solvate thereof, a pharmaceutically acceptable salt thereof, and a prodrug thereof, comprising the following steps:

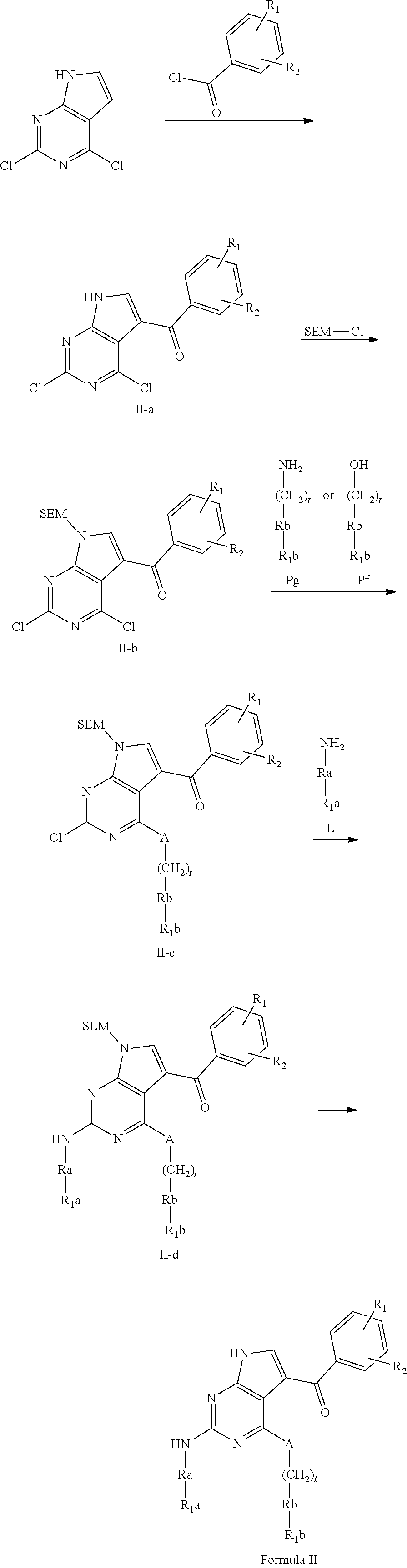

reacting 2,4-dichloro-7H-pyrrolo[2,3-d]pyrimidine as a starting material with

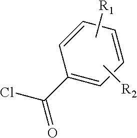

to obtain the compound II-a, wherein the substances in the reaction formula are used in the next steps, the reaction conditions are the same as those in the corresponding steps of formula I to prepare formula II; and the definition of each substituent in the reaction formula is the same as that of each substituent in a synthetic route in the general formula I or the same as that in formula II.

The present invention provides a method for preparing a compound of formula III, including an isomer thereof, a hydrate thereof, a solvate thereof, a pharmaceutically acceptable salt thereof, and a prodrug thereof, comprising the following steps:

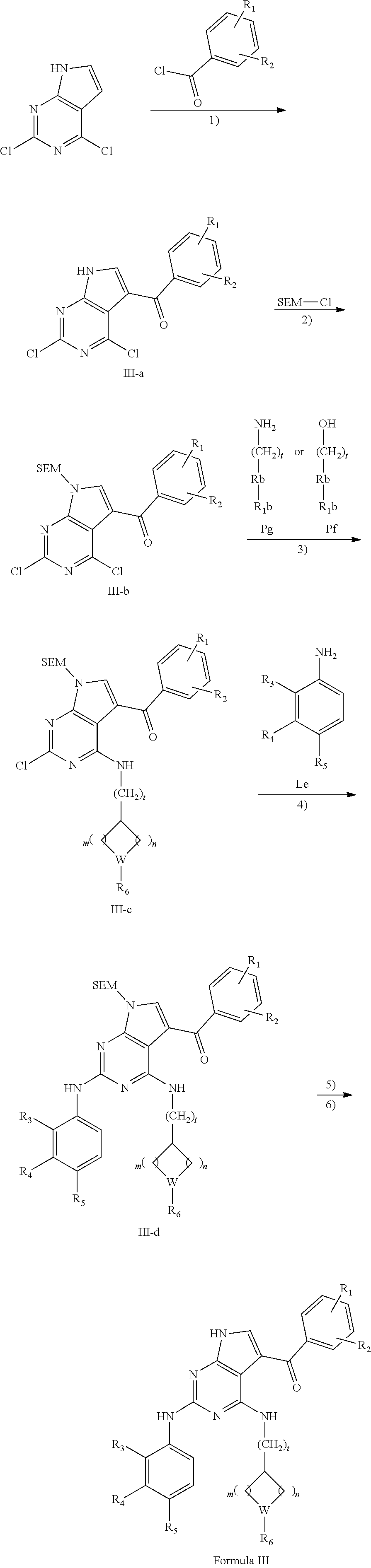

1) reacting 2,4-dichloro-7H-pyrrolo[2,3-d]pyrimidine as a starting material with

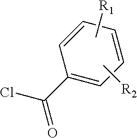

to obtain the compound III-a;
the substances in the reaction formula are used in the following steps 2) and 3), and the reaction conditions are the same as those in the corresponding steps of formula I to prepare formula III-c; and the definition of each substituent in the reaction formula is the same as that of each substituent in a synthetic route in the general formula I or the same as that in formula II; and
4) reacting the compound III-c reacts with

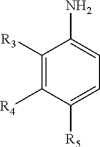

under the heating condition to obtain the compound III-d,
preferably, at 100-130° C.;
performing the reaction in an organic solvent and under the alkaline condition, wherein the alkali and the organic solvent are independently selected from the relevant limitations in the synthetic step 2) of the compound of formula I,
wherein the reaction is Buchwald-hartwig coupling using a palladium catalyst and an organic phosphine ligand, and the palladium catalyst and the organic phosphine ligand are independently selected from relevant limitations in the synthetic step 2) of the compound of formula I; and
in steps 5) and 6), the same preparation method of the corresponding steps of formula I is used to prepare formula III.

The present invention provides a method for preparing a compound of formula IV, including an isomer thereof, a hydrate thereof, a solvate thereof, a pharmaceutically acceptable salt thereof, and a prodrug thereof, comprising the following steps:

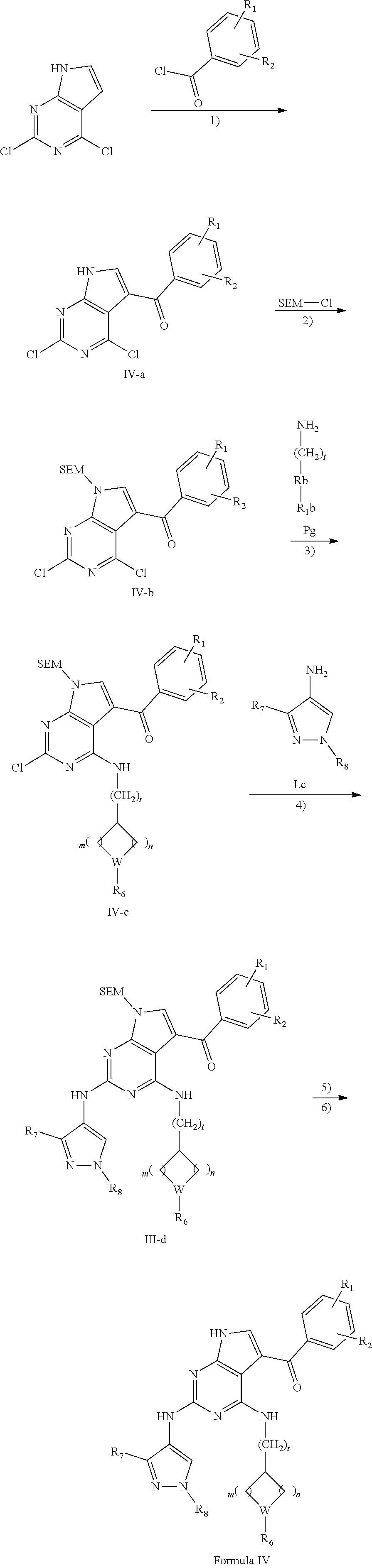

1) reacting 2,4-dichloro-7H-pyrrolo[2,3-d]pyrimidine with

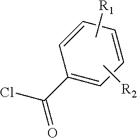

to obtain the compound IV-a,
wherein the substances in the reaction formula are used in the next steps 2) and 3), and the reaction conditions are the same as those in the corresponding steps of formula I to prepare formula IV-c; and the definition of each substituent in the reaction formula is the same as that of each substituent in a synthetic route in the general formula I or the same as that in formula II; and
4) reacting the compound IV-c with

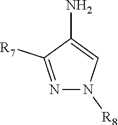

under the heating condition to obtain the compound IV-d,
preferably, at 100-130° C.;
performing the reaction in an organic solvent and under the alkaline condition, wherein the alkali and the organic solvent are independently selected from the relevant limitations in the synthetic step 2) of the compound of formula I,
wherein the reaction is Buchwald-hartwig coupling using a palladium catalyst and an organic phosphine ligand, and the palladium catalyst and the organic phosphine ligand are independently selected from relevant limitations in the synthetic step 2) of the compound of formula I; and
in steps 5) and 6), the same preparation method of the corresponding steps of formula I is used to prepare formula IV.

The present invention further aims to provide a pharmaceutical composition containing an effective dose of the compound, the stereoisomer, or the pharmaceutically acceptable salt thereof of the present invention and one or more pharmaceutically acceptable pharmaceutical excipients. The compound of the present invention can be prepared independently or together with one or more medicinal carriers into different formulations such as a tablet, a pill, a powder, a capsule, a granule, a syrup, an emulsion, a microemulsion, or an injection. Specifically, the compound can be intravenously infused, subcutaneously infused, intramuscularly infused, intraperitoneally infused, transdermally infused, and directly infused into tissues for clinical oral administration, injection, or topical administration.

When the pharmaceutical composition of the present invention is prepared in the form of an oral formulation, an ingredient known in the art may be used as a pharmaceutically acceptable carrier without limitation so long as it does not interfere with the active expression of an active ingredient. The carrier may include, for example, an excipient, a diluent, a disintegrant, a binder, a glidant, a surfactant, an emulsifier, a suspending agent, a diluent, and the like, but is not limited thereto.

When the pharmaceutical composition of the present invention is prepared in the form of an injection, an ingredient known in the art may be used as a pharmaceutically acceptable carrier without limitation so long as it does not interfere with the active expression of an active ingredient. Specifically, the carrier may include, for example, water, saline, a dextrose aqueous solution, a pseudo-sugar aqueous solution, an alcohol, a glycol, an ether (e.g., polyethylene glycol 400), an oil, a fatty acid, a fatty acid ester, a glycerol ester, a surfactant, a suspending agent, an emulsifier, and the like, but is not limited thereto.

In these different formulations, the content of the compound of the present invention may be 0.1%-99.9%. The dose of the compound of the present invention may be 0.001-10,000 mg/kg/0.3 day and may be appropriately adjusted according to clinical needs.

The “pharmaceutically acceptable salt” of the present invention refers to a pharmaceutically acceptable acid or alkali addition salt, a solvate thereof, or a hydrate thereof.

Another objective of the present invention is to provide use of the compound, the stereoisomer thereof, the pharmaceutically acceptable salt thereof, and the pharmaceutical composition thereof in the preparation of an antitumor drug. The tumor is selected from lung cancer, liver cancer, colon cancer, pancreatic cancer, breast cancer, prostate cancer, brain cancer, ovarian cancer, cervical cancer, testicular cancer, kidney cancer, head and neck cancer, lymphoma, melanoma, or leukemia. Further, the tumor is selected from lung cancer, liver cancer, or breast cancer, preferably, lung cancer. A series of tumor cell test results show that the compound of the present invention has broad-spectrum antitumor activity, the IC50 value of the compound is in nanomolar to micromolar level, the activity of the compound is equivalent to that of positive control drugs Brigatinib, Avitinib, and Osimetinib, and the activity of some compounds is superior to that of the positive drugs.

Another objective of the present invention is to provide use of the compound, the stereoisomer thereof, a prodrug thereof, a metabolite thereof, the pharmaceutically acceptable salt thereof, and the pharmaceutical composition thereof in the preparation of an antitumor drug. The tumor is selected from lung cancer, liver cancer, ascitic tumor, metastatic encephaloma, colon cancer, pancreatic cancer, breast cancer, prostate cancer, brain cancer, ovarian cancer, cervical cancer, testicular cancer, kidney cancer, head and neck cancer, lymphoma, melanoma, or leukemia, and preferably selected from lung cancer, liver cancer, breast cancer, ascitic tumor, pancreatic cancer, or metastatic encephaloma, wherein the lung cancer is selected from non-small cell lung cancer or small cell lung cancer, and more preferably selected from non-small cell lung cancer, liver cancer, breast cancer, and ascitic tumor, and the non-small cell lung cancer is lung adenocarcinoma. In a specific embodiment of the present invention, the lung cancer is selected from EGFR-mutant lung cancer, KRAS-mutant lung cancer, HER2-mutant lung cancer, PIK3CA-mutant lung cancer, BRAF-mutant lung cancer, MET gene-mutant lung cancer, ALK gene rearrangement lung cancer, ROS1 gene rearrangement lung cancer, and RET gene rearrangement lung cancer. Further, in a specific embodiment of the present invention, the EGFR-mutant lung cancer is selected from EGFR-Del19-mutant lung cancer, EGFR-L858R-mutant lung cancer, EGFR-C797S-mutant lung cancer, EGFR-C797S/T790M-mutant lung cancer, EGFR-L858R/T790M-mutant lung cancer, EGFR-Del19/T790M-mutant lung cancer, EGFR-L858R/T790M/C797S-mutant lung cancer, EGFR-Del19/T790M/C797S-mutant lung cancer, EGFR D770-N771 ins NPG-mutant lung cancer, or EGFR D770-N771 ins NPG/T790M-mutant lung cancer. In a specific embodiment of the present invention, the EGFR-mutant lung cancer is EGFR Del19/T790M/C797S-mutant lung cancer. In another specific embodiment of the present invention, the EGFR-mutant lung cancer is EGFR D770-N771 ins NPG-mutant lung cancer or EGFR D770-N771 ins NPG/T790M-mutant lung cancer.

Another objective of the present invention is to provide a method for treating a tumor, comprising administering the compound, the stereoisomer thereof, the pharmaceutically acceptable salt thereof, and the pharmaceutical composition thereof with the effective amount to a subject or a patient. The tumor is selected from lung cancer, liver cancer, ascitic tumor, metastatic encephaloma, colon cancer, pancreatic cancer, breast cancer, prostate cancer, brain cancer, ovarian cancer, cervical cancer, testicular cancer, kidney cancer, head and neck cancer, lymphoma, melanoma, or leukemia, preferably selected from lung cancer, liver cancer, breast cancer, ascitic tumor, pancreatic cancer, or metastatic encephaloma, and more preferably selected from non-small cell lung cancer, liver cancer, breast cancer, or ascitic tumor. A series of tumor cell tests show that: the present invention provides a novel-structured compound with an inhibitory activity of EGFR. Compared with the existing similar compounds, the compound of the present invention has a lower inhibition concentration on a tumor cell. The compound of the present invention has a significant inhibitory activity on EGFR D770-N771 ins NPG and EGFR D770-N771 ins NPG/T790M kinases, a very good inhibitory effect on an EGFR-Del19/T790M/C797S kinase, and has a very good inhibitory effect on a KC-0122:Ba/F3EGFR-L858R/T790M/C797S three-mutation cell line and a KC-0116:Ba/F3EGFR-Del19/T790M/C797S three-mutation cell line, and a weaker inhibitory effect on an EGFR wild-type cell line A549 with good selectivity. The compound provided by the present invention has an excellent inhibitory activity on mutant EGFR or EGFR-mutant cancer cells. The compound provided by the present invention has great industrialization and commercialization prospects and market value, and remarkable economic benefits. The compound provided by the present invention has great industrialization and commercialization prospects and market value, and remarkable economic benefits.

Although the preferred embodiments of the present invention have been shown and described herein, it would have been obvious to those skilled in the art that such embodiments are provided by way of examples only. At present, those skilled in the art will think of many variations, changes, and substitutions without departing from the present invention. It should be understood that various alternatives to the embodiments of the present invention described herein may be used in implementing the present invention. It is intended that the following claims define the scope of the present invention and cover the methods and structures, and equivalent forms thereof within the scope of these claims.

### Some Chemical Terms

Unless defined otherwise, all technical and scientific terms used herein have the same meaning as commonly understood by one of ordinary skills belonging to the art of the subject matter of the claims. All patents, patent applications, and publications cited herein are incorporated by reference in their entirety unless otherwise indicated.

It is to be understood that both the foregoing brief description and the following detailed description are exemplary and explanatory only and are not restrictive of the subject matter of the present application. In the present application, the use of the singular also includes the plural unless otherwise specifically indicated. It should also be noted that the use of “or” means “and/or” unless otherwise indicated. Furthermore, the term “comprises” and other forms thereof, such as “includes”, “including”, and “contains” are intended to be non-limiting description.

Definitions for standardized chemical terms can be found in the reference (including Carey and Sundberg “ADVANCED ORGANIC CHEMISTRY 4TH ED”, Vols. A(2000) and B(2001), Plenum Press, New York). Unless otherwise indicated, conventional methods within the technical range of the art, such as mass spectrometry, NMR, and pharmacological methods, are used. Unless specifically defined, the terms used herein in the related description of analytical chemistry, organic synthetic chemistry, and pharmaceuticals and pharmaceutical chemistry are known in the art. Standard techniques can be used in chemical synthesis, chemical analysis, pharmaceutical preparation, formulation and delivery, and treatment of patients. For example, the reaction and purification can be performed using instructions from the manufacturer for use of the kit, or in a manner known in the art, or using the description of the present application. The techniques and methods described above can generally be implemented according to conventional methods well known in the art, as described in various general and more specific references referred to and discussed in the description. In the present description, groups and substituents thereof may be selected by those skilled in the art to provide stable structural moieties and compounds.

When a substituent is described by a conventional chemical formula written from left to right, the substituent also includes a chemically equivalent substituent obtained when the formula is written from right to left.

For example, CH2O is equivalent to OCH2.

The term “compound” of the present application include all stereoisomers, geometric isomers, tautomers, and isotopes. The compound of the present application may be asymmetric, e.g., having one or more stereoisomers. Unless otherwise indicated, all the stereoisomers include, for example, enantiomers and diastereomers. The compound of the present application containing an asymmetrically substituted carbon atom can be isolated in the optically active pure form or in the racemic form. The optically active pure form can be resolved from a racemic mixture or synthesized by using a chiral raw material or a chiral reagent. The compound of the present application further includes the tautomeric form. The tautomeric form is derived from the exchange of one single bond with an adjacent double bond and the concomitant migration of one proton. The compound of the present application further includes all isotopic atoms, whether in an intermediate or a final compound. The isotopic atoms include atoms having the same number of atoms but different mass numbers. For example, the isotopes of hydrogen include tritium and deuterium. That is, the compound of the present application includes compounds in which some or all hydrogens (H) are substituted by tritium (T) and/or deuterium (D), and further includes compounds with some or all ¹²C substituted by ¹³C and/or ¹⁴C, and compounds with substitutions between other isotopes (e.g. N, O, P, and S), such as ¹⁴N and ¹⁵N, ¹⁸O and ¹⁷O, ³¹P and ³²P, ³⁵S and ³⁶S, and the like. The compound described herein may have one or more stereoisomeric centers, and each stereoisomeric center may exist in the R or S configuration or a combination thereof. Similarly, the compound described herein may have one or more double bonds, and each double bond may be present in the E (trans) or Z (cis) configuration or a combination thereof. A particular stereoisomer, structural isomer, diastereoisomer, enantiomer or epimer is understood to encompass all possible isomers, such as a stereoisomer, a structural isomer, a diastereoisomer, an enantiomer, or an epimer, and a mixture thereof. Thus, the compound described herein includes all configurationally distinct stereoisomers, structural isomers, diastereomers, enantiomers, or epimers, and a corresponding mixture thereof. Techniques for converting a particular stereoisomer or keeping an original state of the particular stereoisomer, and resolving a mixture of the stereoisomer are well known in the art, and those skilled in the art will be able to select suitable methods for the particular situation.

The term “optional/random” or “optionally/randomly” means that the subsequently described event or circumstance may or may not occur. The description includes instances where the event or circumstance occurs and does not occur.

The C₁₋₃ or C1-3 used herein means that the moiety has 1-3 carbon atoms, i.e., the group contains 1 carbon atom, 2 carbon atoms, and 3 carbon atoms. Therefore, for example, “C1-3 alkyl” refers to alkyl having 1-3 carbon atoms, i.e., the alkyl is selected from methyl, ethyl, propyl, and isopropyl.

The term “alkyl” used herein, alone or in combination, refers to an optionally substituted straight-chain or optionally substituted branched-chain aliphatic hydrocarbon. The “alkyl” herein preferably may have from 1 to about 20 carbon atoms, for example, from 1 to about 10 carbon atoms, from 1 to about 8 carbon atoms, from 1 to about 6 carbon atoms, from 1 to about 4 carbon atoms, or from 1 to about 3 carbon atoms. Examples of the alkyl herein include, but are not limited to, methyl, ethyl, n-propyl, isopropyl, and the like.

The “alkyl” used in combination herein includes alkyl in combination with other groups, such as alkyl in alkoxy.

The term “halogen” used herein, alone or in combination, is selected from F, Cl, Br, and I.

As used herein, the term “treating” and other similar synonyms include alleviating, reducing, or ameliorating a symptom of a disease or disorder, and inhibiting a disease or disorder, e.g., arresting the development of a disease or disorder, ameliorating a disease or disorder, alleviating a symptom caused by a disease or disorder, or halting a symptom of a disease or disorder, preventing other symptoms, and relieving or preventing an underlying metabolic cause leading to the symptom. In addition, the term encompasses a prophylactic purpose. The term further comprises obtaining a therapeutic effect and/or a prophylactic effect. The therapeutic effect refers to curing or relieving an underlying disease being treated. In addition, curing or relieving one or more physiological symptoms associated with an underlying disease is also a therapeutic effect, e.g., an improvement in a patient's condition is observed, although the patient may still be affected by the underlying disease. For the prophylactic effect, the composition may be administered to a patient at risk of developing a particular disease, or to a patient having one or more physiological symptoms of the disease, even if a diagnosis of the disease has not yet been made.

The term “effective amount”, “therapeutically effective amount”, or “pharmaceutically effective amount” used herein refers to the amount of at least one active substance (e.g., the compound of the present application) that is sufficient to alleviate one or more symptoms of the disease or disorder being treated to some extent after administration. The result may be a reduction and/or alleviation of signs, symptoms, or causes, or any other desired change in a biological system. For example, the “effective amount” for treatment is the amount of the composition containing the compound disclosed herein that is clinically necessary to provide an obvious effect for alleviating a disorder. The effective amount suitable in any individual case may be determined using techniques such as a dose escalation test.

The term “acceptable” used herein with respect to a formulation, a composition, or an ingredient means that there is no long-term deleterious effect on the general health of a subject being treated.

The term “pharmaceutically acceptable” used herein refers to a substance (e.g., a carrier or a diluent) that does not affect the biological activity or properties of the compound of the present application. Besides, the substance is relatively non-toxic, i.e., the substance can be administered to an individual without causing an undesirable biological response or interacting in an undesirable manner with any component contained in the composition.

The term “pharmaceutical composition” used herein refers to a mixture of the compound of the present application and at least one pharmaceutically acceptable substance. The pharmaceutically acceptable substance includes, but is not limited to, a carrier, a stabilizer, a diluent, a dispersing agent, a suspending agent, a thickening agent, and/or an excipient.

The term “carrier” used herein refers to a relatively non-toxic substance that facilitates the introduction of the compound of the present application into a cell or a tissue.

The term “pharmaceutically acceptable salt” used herein refers to a salt that retains the biological effectiveness of a free acid and a free alkali of the specified compound and has no adverse effects biologically or in other aspects. The compound of the present application further comprises a pharmaceutically acceptable salt. The pharmaceutically acceptable salt refers to the form in which an alkali group in a parent compound is converted to a salt. The pharmaceutically acceptable salt includes, but is not limited to, an inorganic acid or organic acid salt of the alkali group such as an amino group. The pharmaceutically acceptable salt of the present application may be synthesized from the parent compound by reacting the alkali group in the parent compound with 1-4 equivalents of an acid in a solvent system. Suitable salts are listed in Remingtong's Pharmaceutical Scicences, 17th ed., Mack Publishing Company, Easton, Pa., 1985, p. 1418 and Journal of Pharmaceutical Science, 66, 2(1977).

Unless otherwise indicated, the salt in the present application refer to an acid salt formed with an organic/inorganic acid, and an alkali salt formed with an organic/inorganic alkali.

The “EGFR” refers to the epidermal growth factor receptor.

For those skilled in the art, “EGFR mutation-negative” generally means that EGFR gene mutation is not detected according to gene detection methods usually for clinical diagnosis. An EGFR mutation status can be detected by various methods. A DNA mutation detection is the first method for detecting the EGFR status. Various DNA mutation detection analyses can be used for detecting the EGFR mutation status of tumor cells. The most common EGFR mutations of patients with non-small cell lung cancer are exon 19 deletion and exon 21 mutation. Direct DNA sequencing of exons 18-21 (or exons 19 and 21 only) is a reasonable choice.

The “advanced stage” means staging non-small cell lung cancer on the basis of the extent of the pathological changes and complications, e.g., stage III-IV non-small cell lung cancer according to the TNM classification method in the lung cancer staging system of the AJCC cancer staging manual. In some embodiments, advanced-stage non-small cell lung cancer is IIIB—IV non-small cell lung cancer.

As used herein, unless otherwise indicated, the doses and ranges provided herein are calculated on the basis of the molecular weight of the compound of the present application in its free alkali form.

### BRIEF DESCRIPTION OF THE DRAWINGS

FIG. 1 shows a tumor volume curve of mice in each group; and

FIG. 2 shows a tumor weight curve of mice in each group

### DETAILED DESCRIPTION OF EMBODIMENTS

In order to more clearly illustrate the present invention, a series of examples are listed herein. The following examples are illustrative and should not be construed as a limitation to the present invention.

The present invention provides methods for preparing the corresponding compounds. The compounds described herein can be prepared using various synthetic methods, including those referred to in the examples below. The compound, the pharmaceutically acceptable salt, the isomer, or the hydrate thereof of the present invention, may be synthesized by using the methods described below and the synthetic methods known in the art of organic chemical synthesis, or by variations of these methods as understood by those skilled in the art. The preferred method includes, but is not limited to, the methods described below.

### Example 1

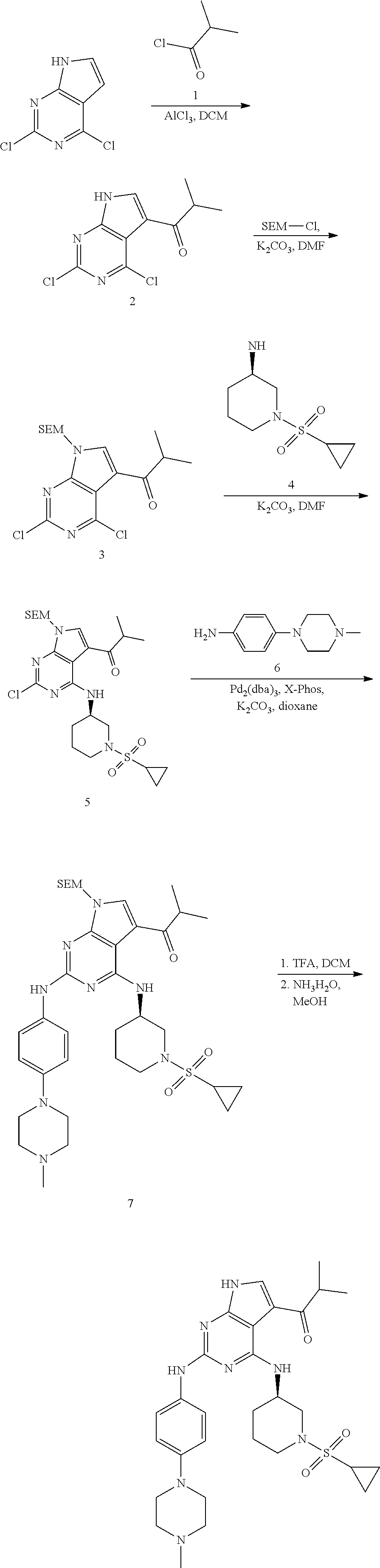

5.0 g of 2,4-dichloro-7H-pyrrolo[2,3-d]pyrimidine and 100 mL of dichloromethane were added into a 250-mL round-bottom flask, 14.15 g of aluminum trichloride was added under stirring in an ice-water bath, stirring was performed for 0.5 h, and 3.5 mL of isobutyryl chloride (compound 1) diluted with 50 mL of dichloromethane was added dropwise. After the addition, the mixture was heated and subjected to a reflux reaction for 12 h. An obtained system was poured into ice water and stirred for 1 h. An obtained aqueous phase was extracted with dichloromethane and obtained organic phases were combined. The organic phase was washed with a saturated salt solution, dried over anhydrous sodium sulfate, and concentrated to dryness under reduced pressure. An obtained residue was subjected to silica gel column chromatography (petroleum ether:ethyl acetate=10:1) to obtain 2,4-dichloro-5-(2-methylacetonyl)-7H-pyrrolo[2,3-d]pyrimidine (intermediate 2).

2 g of the intermediate 2, 30 mL of N,N-dimethylformamide, and 2.65 g of potassium carbonate were added to a 100-mL round bottom flask, 1.7 mL of 2-(trimethylsilyl)ethoxymethyl chloride diluted with 20 mL of N,N-dimethylformamide was added dropwise while the temperature of an oil bath was controlled at 40° C., and after the addition was completed, an obtained mixture was stirred for 4 h while the temperature was maintained at 40° C. An obtained system was poured into water, an obtained aqueous phase was extracted with ethyl acetate, and obtained organic phases were combined. The organic phase was washed with a saturated salt solution, dried over anhydrous sodium sulfate, and concentrated to dryness under reduced pressure. An obtained residue was subjected to silica gel column chromatography (petroleum ether:ethyl acetate=15:1) to obtain 2,4-dichloro-5-(2-methylacetonyl)-7-(trimethylsilyl ethoxymethyl)-7H-pyrrolo[2,3-d]pyrimidine (intermediate 3).

200 mg of the intermediate 3, 10 mL of N,N-dimethylformamide, 186 mg of potassium carbonate, and (R)-1-cyclopropylsulfonyl-3-aminopiperidine (compound 4) were added into a 50-mL eggplant-shaped flask, and an obtained mixture was stirred in an oil bath at 90° C. for 3 h. The N,N-dimethylformamide was evaporated under reduced pressure, 20 mL of water was added into an obtained residue, the residue was ultrasonically stirred for 10 min and subjected to suction filtration, and a filter cake was dried to obtain (R)-2-chloro-4-(1-cyclopropylsulfonyl-3-piperidinamino)-5-(2-methylpropionyl)-7-(trimethylsilylethoxymethyl)-7H-pyrrolo[2,3-d]pyrimidine (intermediate 5).

150 mg of the intermediate 5, 45 mg of 4-(4-methyl-1-piperazin)-1-aniline (compound 6), 27 mg of Pd₂(dba)₃, 43 mg of X-Phos, 124 mg of potassium carbonate, and 10 mL of 1,4-dioxane were added into a 100-mL eggplant-shaped flask, and an obtained mixture was stirred in an oil bath at 110° C. for 4 h under the nitrogen atmosphere. The mixture was concentrated to dryness under reduced pressure. An obtained residue was subjected to silica gel column chromatography (dichloromethane:methanol=20:1) to obtain 2-(4-(4-methyl-1-piperazin)-1-phenylamino)-4-(tetrahydropyran-4-amino)-5-(2-methylpropanoyl)-7-(trimethylsilylethoxymethyl)-7H-pyrrolo[2,3-d]pyrimidine (intermediate 7).

110 mg of the intermediate 5, 5 mL of dichloromethane, and 2 mL of trifluoroacetic acid were added into a 100-mL eggplant-shaped flask, and an obtained mixture was stirred at room temperature for 2 h. The dichloromethane and the trifluoroacetic acid were evaporated under reduced pressure, 10 mL of methanol and 4 mL of ammonium hydroxide were added, and an obtained mixture was stirred at room temperature for 4 h. The methanol and the ammonium hydroxide were evaporated under reduced pressure. An obtained residue was subjected to silica gel column chromatography (dichloromethane:methanol=20:1) to obtain (R)-2-(4-(4-methyl-1-piperazin)-1-phenylamino)-4-(1-cyclopropylsulfonyl-3-piperidinamino)-5-(2-methylpropanoyl)-7H-pyrrolo[2,3-d]pyrimidine. ¹H NMR (600 MHz, DMSO-d₆) δ 12.01 (s, 1H), 9.09 (d, J=7.8 Hz, 1H), 8.64 (s, 1H), 8.08 (s, 1H), 7.61 (d, J=9.0 Hz, 2H), 6.84 (d, J=9.1 Hz, 2H), 4.37-4.24 (m, 1H), 3.70-3.61 (m, 1H), 3.55-3.47 (m, 1H), 3.41-3.37 (m, 1H), 3.33-3.31 (m, 1H), 3.11-2.98 (m, 6H), 2.63-2.59 (m, 1H), 2.53-2.52 (m, 2H), 2.48-2.47 (m, 1H), 2.24 (s, 3H), 2.03-1.87 (m, 2H), 1.70-1.54 (m, 2H), 1.11 (d, J=6.8 Hz, 6H), 0.97-0.89 (m, 4H). MS/[M+H]+:581.36

### Example 2

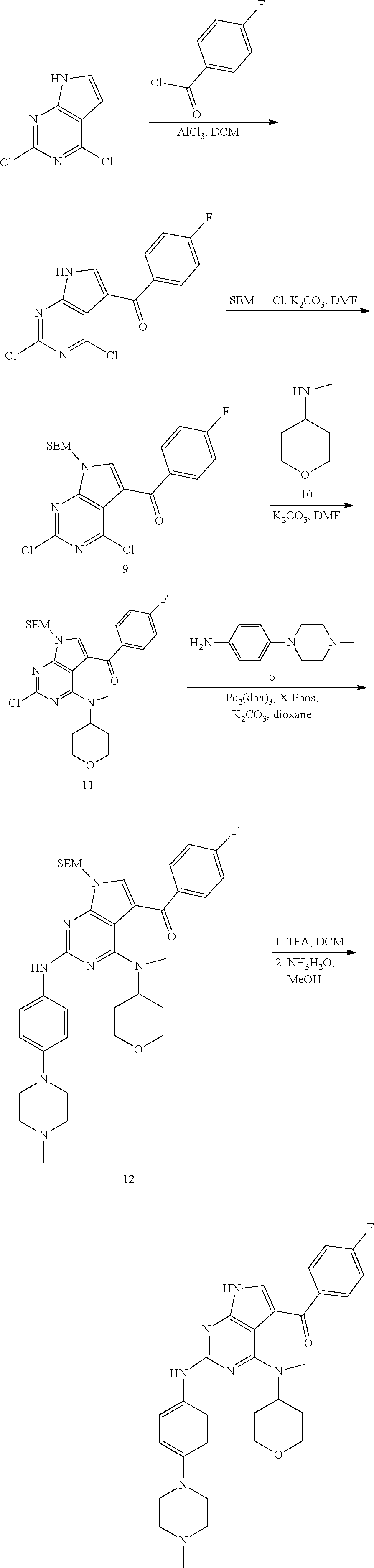

The preparation method used in example 2 was the same as that in example 1. ¹H NMR (600 MHz, DMSO-d₆) δ 11.88 (s, 1H), 8.69 (s, 1H), 7.96 (dd, J=8.5, 5.7 Hz, 2H), 7.62 (d, J=8.9 Hz, 2H), 7.37 (t, J=8.7 Hz, 2H), 7.32 (d, J=2.5 Hz, 1H), 6.86 (d, J=9.0 Hz, 2H), 4.41-4.27 (m, 1H), 3.93-3.78 (m, 2H), 3.21-3.11 (m, 2H), 3.10-3.00 (m, 4H), 2.79 (s, 3H), 2.49-2.45 (m, 4H), 2.23 (s, 3H), 1.76-1.63 (m, 2H), 1.56-1.43 (m, 2H). MS/[M+H]⁺:544.27

### Example 3

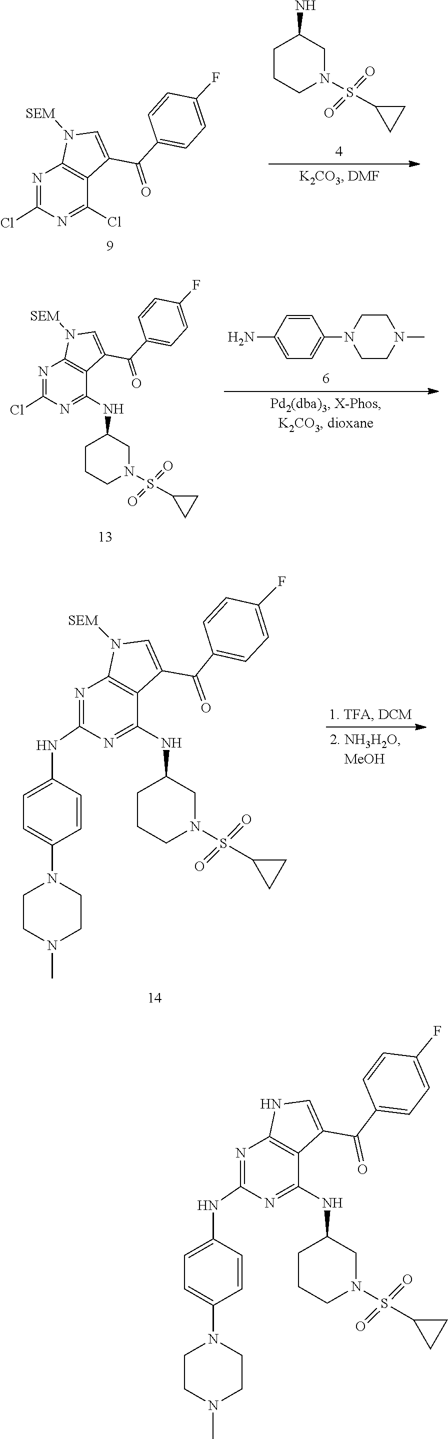 [SMILES: C[Si](C)(C)CCOCn1cc(C(=O)c2ccc(F)cc2)c2c(Cl)nc(Cl)nc21 ; N[C@@H]1CCCN(S(=O)(=O)C2CC2)C1 ; C[Si](C)(C)CCOCn1cc(C(=O)c2ccc(F)cc2)c2c(N[C@@H]3CCCN(S(=O)(=O)C4CC4)C3)nc(Cl)nc21 ; CN1CCN(c2ccc(N)cc2)CC1 ; CN1CCN(c2ccc(Nc3nc(N[C@@H]4CCCN(S(=O)(=O)C5CC5)C4)c4c(C(=O)c5ccc(F)cc5)cn(COCC[Si](C)(C)C)c4n3)cc2)CC1 ; CN1CCN(c2ccc(Nc3nc(N[C@@H]4CCCN(S(=O)(=O)C5CC5)C4)c4c(C(=O)c5ccc(F)cc5)c[nH]c4n3)cc2)CC1]

The preparation method used in example 3 was the same as that in example 1. ¹H NMR (600 MHz, DMSO-d₆) δ 12.21 (s, 1H), 8.85 (d, J=7.8 Hz, 1H), 8.74 (s, 1H), 7.84 (dd, J=8.5, 5.6 Hz, 2H), 7.63 (d, J=8.9 Hz, 2H), 7.48 (s, 1H), 7.36 (t, J=8.8 Hz, 2H), 6.86 (d, J=9.0 Hz, 2H), 4.47-4.27 (m, 1H), 3.70-3.56 (m, 1H), 3.33-3.31 (m, 1H), 3.17-3.07 (m, 2H), 3.07-3.00 (m, 4H), 2.62-2.58 (m, 1H), 2.55-2.52 (m, 4H), 2.26 (s, 3H), 2.04-1.88 (m, 2H), 1.73-1.58 (m, 2H), 0.99-0.88 (m, 4H). MS/[M+H]⁺:633.31

### Example 4

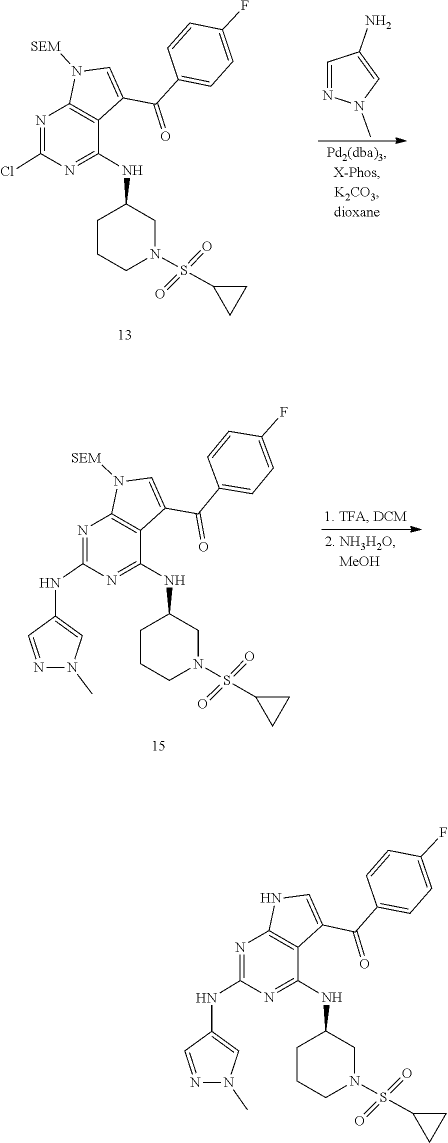

The preparation method used in example 4 was the same as that in example 1. ¹H NMR (600 MHz, DMSO-d₆) δ 12.15 (s, 1H), 9.04-8.71 (m, 2H), 7.87 (s, 1H), 7.84 (dd, J=8.1, 5.7 Hz, 2H), 7.54-7.39 (n, J=2.3 Hz, 2H), 7.36 (t, J=8.7 Hz, 2H), 4.42-4.20 (i, 1H), 3.78 (s, 3H), 3.47-3.38 (i, 1H), 3.34-3.29 (m, 1H), 3.13-3.04 (m, 1H), 3.03-2.94 (m, 1H), 2.63-2.59 (m, 1H), 2.09-1.88 (m, 2H), 1.74-1.58 (m, 2H), 0.99-0.87 (m, 4H).MS/[M+H]⁺:539.24

The following compounds of examples 8-116 were synthesized with reference to the specific synthetic steps of example 1, guided by the synthetic route of general formula I.

| | | | Mass |
|---|---|---|---|
| | | | spectrum/actual |
| | | | measurement |
| Example | Structure | H-NMR | [M + H]⁺ |
| 8 | 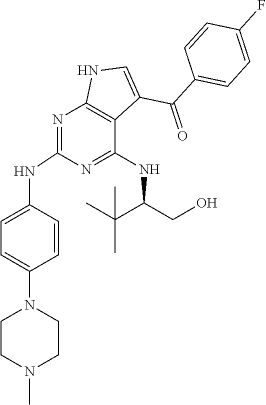 [SMILES: CN1CCN(c2ccc(Nc3nc(N[C@@H](CO)C(C)(C)C)c4c(C(=O)c5ccc(F)cc5)c[nH]c4n3)cc2)CC1] | ¹H NMR (600 MHz, DMSO-d₆) δ 12.12 (s, 1H), 8.79 (d, J = 9.3 Hz, 1H), 8.62 (s, 1H), 7.84 (dd, J = 8.3, 5.7 Hz, 2H), 7.67 (d, J = 8.9 Hz, 2H), 7.43 (s, 1H), 7.35 (t, J = 8.7 Hz, 2H), 6.84 (d, J = 8.9 Hz, 2H), 4.58 (s, 1H), 4.33-4.22 (m, 1H), 3.80-3.70 (m, 1H), 3.58-3.49 (m, 1H), 3.15-2.97 (m, 4H), 2.55-2.52 (m, 4H), 2.26 (s, 3H), 1.04 (s, 9H). | 546.28 |
| 9 | 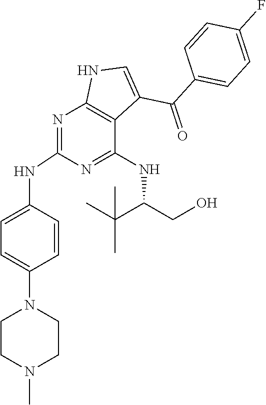 [SMILES: CN1CCN(c2ccc(Nc3nc(N[C@H](CO)C(C)(C)C)c4c(C(=O)c5ccc(F)cc5)c[nH]c4n3)cc2)CC1] | ¹H NMR (600 MHz, DMSO-d₆) δ 12.11 (s, 1H), 8.79 (d, J = 9.2 Hz, 1H), 8.62 (s, 1H), 7.91-7.78 (m, 2H), 7.67 (d, J = 8.5 Hz, 2H), 7.43 (s, 1H), 7.35 (t, J = 8.4 Hz, 2H), 6.84 (d, J = 8.5 Hz, 2H), 4.58 (s, 1H), 4.33-4.22 (m, 1H), 3.80-3.69 (m, 1H), 3.59-3.49 (m, 1H), 3.14-2.99 (m, 4H), 2.54-2.52 (m, 4H), 2.26 (s, 3H), 1.04 (s, 9H). | 546.27 |
| 10 | 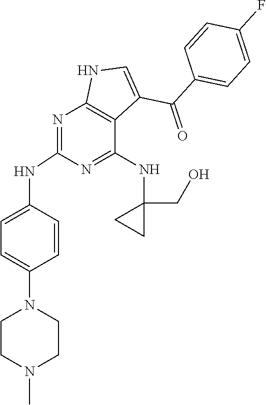 [SMILES: CN1CCN(c2ccc(Nc3nc(NC4(CO)CC4)c4c(C(=O)c5ccc(F)cc5)c[nH]c4n3)cc2)CC1] | ¹H NMR (600 MHz, DMSO-d₆) δ 12.13 (s, 1H), 9.00 (s, 1H), 8.72 (s, 1H), 7.82 (dd, J = 8.5, 5.6 Hz, 2H), 7.77 (d, J = 8.9 Hz, 2H), 7.46 (s, 1H), 7.36 (t, J = 8.8 Hz, 2H), 6.86 (d, J = 9.0 Hz, 2H), 4.97-4.66 (m, 1H), 3.75-3.60 (m, 2H), 3.14-3.00 (m, 4H), 2.59-2.53 (m, 4H), 2.29 (s, 3H), 0.97- 0.89 (m, 2H), 0.82-0.76 (m, 2H). | 516.22 |
| 11 | 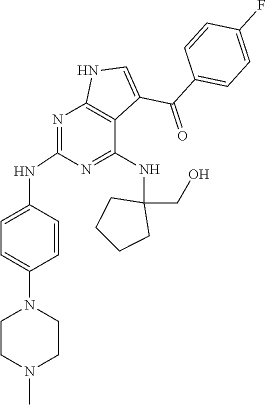 [SMILES: CN1CCN(c2ccc(Nc3nc(NC4(CO)CCCC4)c4c(C(=O)c5ccc(F)cc5)c[nH]c4n3)cc2)CC1] | ¹H NMR (600 MHz, DMSO-d₆) δ 12.10 (s, 1H), 8.82 (s, 1H), 8.53 (s, 1H), 7.82 (dd, J = 8.6, 5.6 Hz, 2H), 7.61 (d, J = 9.0 Hz, 2H), 7.40 (s, 1H), 7.35 (t, J = 8.8 Hz, 2H), 6.85 (d, J = 9.0 Hz, 2H), 4.83 (t, J = 5.2 Hz, 1H), 3.82 (d, J = 5.2 Hz, 2H), 3.13- 2.98 (m, 4H), 2.54-2.51 (m, 4H), 2.25 (s, 3H), 2.13-2.04 (m, 2H), 1.94-1.86 (m, 2H), 1.76-1.59 (m, 4H). | 544.26 |
| 12 | 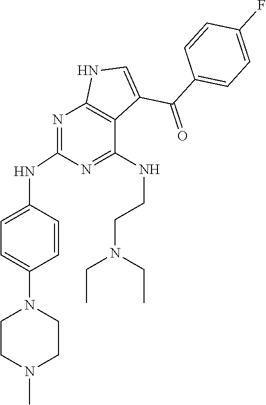 [SMILES: CCN(CC)CCNc1nc(Nc2ccc(N3CCN(C)CC3)cc2)nc2[nH]cc(C(=O)c3ccc(F)cc3)c12] | ¹H NMR (600 MHz, DMSO-d₆) δ 12.12 (s, 1H), 8.69 (t, J = 5.4 Hz, 1H), 8.63 (s, 1H), 7.86-7.79 (m, 2H), 7.64 (d, J = 9.0 Hz, 2H), 7.43 (s, 1H), 7.35 (t, J = 8.8 Hz, 2H), 6.85 (d, J = 9.1 Hz, 2H), 3.70-3.61 (m, 2H), 3.08-3.02 (m, 4H), 2.84-2.71 (m, 2H), 2.71-2.57 (m, 4H), 2.55-2.52 (m, 4H), 2.24 (s, 3H), 1.01 (t, J = 7.0 Hz, 6H). | 545.31 |
| 13 | 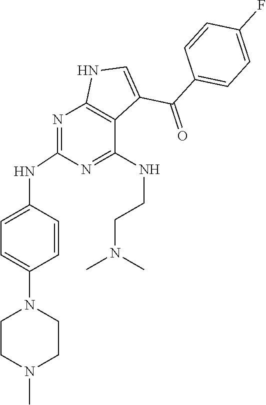 [SMILES: CN(C)CCNc1nc(Nc2ccc(N3CCN(C)CC3)cc2)nc2[nH]cc(C(=O)c3ccc(F)cc3)c12] | ND | 517.28 |
| 14 | 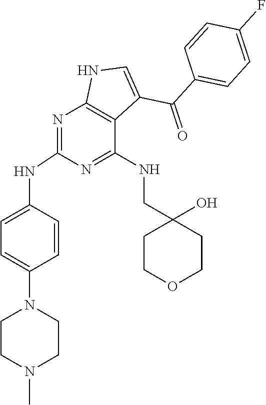 [SMILES: CN1CCN(c2ccc(Nc3nc(NCC4(O)CCOCC4)c4c(C(=O)c5ccc(F)cc5)c[nH]c4n3)cc2)CC1] | ¹H NMR (600 MHz, DMSO-d₆) δ 12.13 (s, 1H), 8.84 (t, J = 5.5 Hz, 1H), 8.73 (s, 1H), 7.86-7.82 (m, 2H), 7.76-7.69 (m, 2H), 7.45 (d, J = 2.6 Hz, 1H), 7.37-7.34 (m, 2H), 6.92 (d, J = 9.1 Hz, 2H), 4.73 (s, 1H), 3.70-3.49 (m, 10H), 2.81 (s, 3H), 2.52-2.52 (m, 4H), 1.65-1.61 (m, 2H), 1.54-1.50 (m, 2H). | 560.27 |
| 15 | 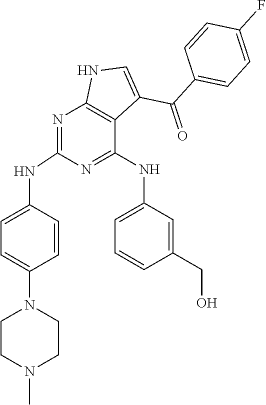 [SMILES: CN1CCN(c2ccc(Nc3nc(Nc4cccc(CO)c4)c4c(C(=O)c5ccc(F)cc5)c[nH]c4n3)cc2)CC1] | ¹H NMR (600 MHz, DMSO-d₆) δ 12.43 (s, 1H), 11.23 (s, 1H), 9.74 (s, 1H), 8.99 (s, 1H), 8.01 (d, J = 5.4 Hz, 1H), 7.95-7.86 (m, 2H), 7.68 (d, J = 8.7 Hz, 2H), 7.66- 7.60 (m, 2H), 7.39 (t, J = 8.8 Hz, 2H), 7.31 (t, J = 7.8 Hz, 1H), 7.01 (d, J = 7.5 Hz, 1H), 6.97 (d, J = 9.0 Hz, 2H), 5.39-5.01 (m, 1H), 4.52 (s, 2H), 3.80-3.47 (m, 4H), 3.24-3.15 (m, 2H), 3.00-2.83 (m, 5H). | 552.25 |
| 16 | 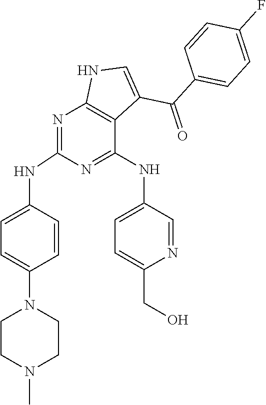 [SMILES: CN1CCN(c2ccc(Nc3nc(Nc4ccc(CO)nc4)c4c(C(=O)c5ccc(F)cc5)c[nH]c4n3)cc2)CC1] | ¹H NMR (600 MHz, DMSO-d₆) δ 12.49 (s, 1H), 11.24 (s, 1H), 9.03 (s, 1H), 8.91 (s, 1H), 8.47 (dd, J = 8.5, 2.3 Hz, 1H), 7.93-7.89 (m, 2H), 7.65 (s, 1H), 7.62 (d, J = 8.5 Hz, 2H), 7.44 (d, J = 8.5 Hz, 1H), 7.41-7.37 (m, 2H), 6.89 (d, J = 9.1 Hz, 2H), 5.35 (t, J = 5.6 Hz, 1H), 4.56 (d, J = 5.2 Hz, 2H), 3.13-3.05 (m, 4H), 2.52-2.52 (m, 4H), 2.27 (s, 3H). | 553.24 |
| 17 | 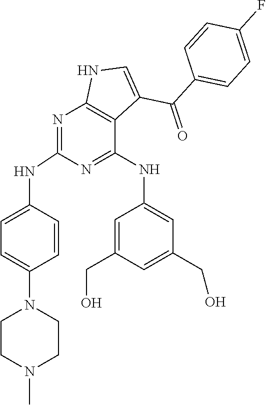 [SMILES: CN1CCN(c2ccc(Nc3nc(Nc4cc(CO)cc(CO)c4)c4c(C(=O)c5ccc(F)cc5)c[nH]c4n3)cc2)CC1] | ND | 582.26 |
| 18 | 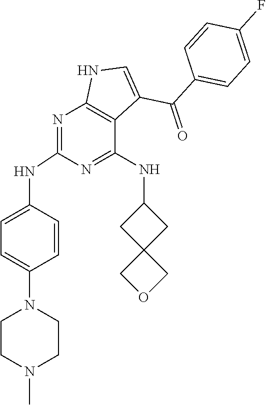 [SMILES: CN1CCN(c2ccc(Nc3nc(NC4CC5(COC5)C4)c4c(C(=O)c5ccc(F)cc5)c[nH]c4n3)cc2)CC1] | ¹H NMR (600 MHz, DMSO-d₆) δ 12.12 (s, 1H), 8.82 (d, J = 7.2 Hz, 1H), 8.68 (s, 1H), 7.91-7.80 (m, 2H), 7.70 (d, J = 9.0 Hz, 2H), 7.43 (d, J = 2.8 Hz, 1H), 7.38- 7.31 (m, 2H), 6.87 (d, J = 9.1 Hz, 2H), 4.68-4.53 (m, 3H), 3.52-3.48 (m, 2H), 3.23-3.00 (m, 4H), 2.74 (s, 3H), 2.52-2.52 (m, 4H), 2.28-2.21 (m, 2H), 1.87-1.78 (m, 2H). | 542.26 |
| 19 | 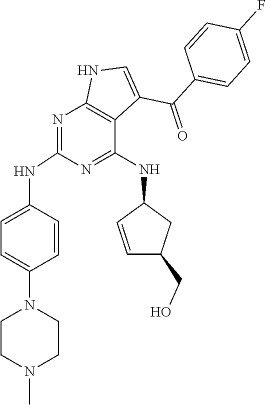 [SMILES: CN1CCN(c2ccc(Nc3nc(N[C@@H]4C=C[C@H](CO)C4)c4c(C(=O)c5ccc(F)cc5)c[nH]c4n3)cc2)CC1] | ¹H NMR (600 MHz, DMSO-d₆) δ 12.17 (s, 1H), 8.77 (s, 1H), 8.72 (d, J = 7.7 Hz, 1H), 7.86-7.81 (m, 2H), 7.77-7.70 (m, 2H), 7.45 (s, 1H), 7.35 (t, J = 8.8 Hz, 2H), 6.91 (d, J = 9.1 Hz, 2H), 6.03-5.97 (m, 1H), 5.97-5.89 (m, 1H), 5.30-5.23 (m, 1H), 4.72-4.68 (m, 2H), 3.47-3.38 (m, 5H), 3.25-3.18 (m, 4H), 2.78 (s, 3H), 2.57- 2.51 (m, 2H), 1.49-1.37 (m, 1H). | 542.26 |
| 20 | 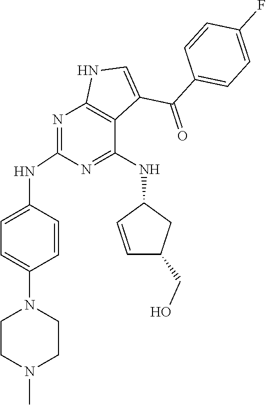 [SMILES: CN1CCN(c2ccc(Nc3nc(N[C@H]4C=C[C@@H](CO)C4)c4c(C(=O)c5ccc(F)cc5)c[nH]c4n3)cc2)CC1] | ¹H NMR (600 MHz, DMSO-d₆) δ 12.16 (s, 1H), 8.79 (s, 1H), 8.72 (d, J = 7.6 Hz, 1H), 7.82 (dd, J = 8.3, 5.7 Hz, 2H), 7.74 (d, J = 9.0 Hz, 2H), 7.45 (s, 1H), 7.35 (t, J = 8.8 Hz, 2H), 6.92 (d, J = 8.9 Hz, 2H), 6.05-5.96 (m, 1H), 5.96-5.87 (m, 1H), 5.35-5.20 (m, 1H), 4.99-4.49 (m, 2H), 3.49-3.37 (m, 4H), 3.32-3.15 (m, 4H), 2.85 (s, 3H), 2.82-2.75 (m, 1H), 2.58-2.51 (m, 2H), 1.48-1.37 (m, 1H). | 542.26 |
| 21 | 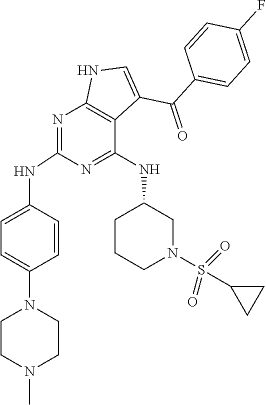 [SMILES: CN1CCN(c2ccc(Nc3nc(N[C@H]4CCCN(S(=O)(=O)C5CC5)C4)c4c(C(=O)c5ccc(F)cc5)c[nH]c4n3)cc2)CC1] | ¹H NMR (600 MHz, DMSO-d₆) δ 12.21 (s, 1H), 8.85 (d, J = 7.9 Hz, 1H), 8.74 (s, 1H), 7.87-7.81 (m, 2H), 7.63 (d, J = 9.0 Hz, 2H), 7.47 (s, 1H), 7.39-7.32 (m, 2H), 6.85 (d, J = 9.1 Hz, 2H), 4.42-4.32 (m, 1H), 3.68-3.58 (m, 1H), 3.33-3.31 (m, 1H), 3.16-3.07 (m, 2H), 3.07-2.97 (m, 4H), 2.63-2.58 (m, 1H), 2.49-2.46 (m, 4H), 2.23 (s, 3H), 2.04-1.88 (m, 2H), 1.72- 1.57 (m, 2H), 0.99-0.88 (m, 4H). | 633.31 |
| 22 | 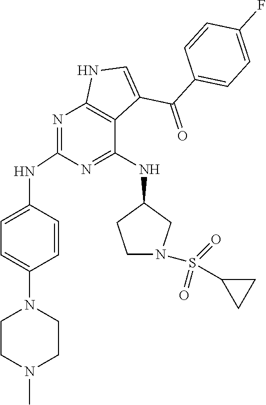 [SMILES: CN1CCN(c2ccc(Nc3nc(N[C@@H]4CCN(S(=O)(=O)C5CC5)C4)c4c(C(=O)c5ccc(F)cc5)c[nH]c4n3)cc2)CC1] | ¹H NMR (600 MHz, DMSO-d₆) δ 12.24 (s, 1H), 8.93 (d, J = 6.1 Hz, 1H), 8.78 (s, 1H), 7.84 (dd, J = 8.4, 5.7 Hz, 2H), 7.64 (d, J = 8.9 Hz, 2H), 7.49 (s, 1H), 7.36 (t, J = 8.8 Hz, 2H), 6.87 (d, J = 9.0 Hz, 2H), 4.80-4.57 (m, 1H), 3.84-3.73 (m, 1H), 3.60-3.45 (m, 2H), 3.33-3.31 (m, 1H), 3.11-2.98 (m, 4H), 2.73-2.64 (m, 1H), 2.49-2.46 (m, 4H), 2.43-2.39 (m, 1H), 2.24 (s, 3H), 2.09-2.01 (m, 1H), 0.98-0.87 (m, 4H). | 619.29 |
| 23 | 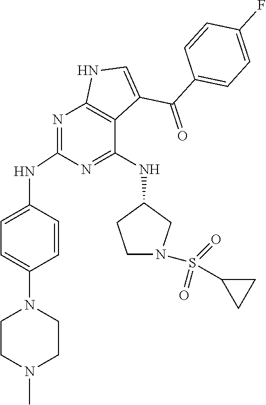 [SMILES: CN1CCN(c2ccc(Nc3nc(N[C@H]4CCN(S(=O)(=O)C5CC5)C4)c4c(C(=O)c5ccc(F)cc5)c[nH]c4n3)cc2)CC1] | ¹H NMR (600 MHz, DMSO-d₆) δ 9.05 (s, 1H), 8.98 (d, J = 6.2 Hz, 1H), 7.98-7.93 (m, 2H), 7.87 (s, 1H), 7.80 (d, J = 8.9 Hz, 2H), 7.51-7.46 (m, 2H), 6.97 (d, J = 9.1 Hz, 2H), 4.85-4.74 (m, 1H), 3.92-3.85 (m, 1H), 3.66-3.58 (m, 2H), 3.43-3.43 (m, 1H), 3.21-3.09 (m, 4H), 2.83-2.76 (m, 1H), 2.59-2.56 (m, 4H), 2.55-2.50 (m, 1H), 2.34 (s, 3H), 2.19-2.10 (m, 1H), 1.06- 0.99 (m, 4H). | 618.73 |
| 24 | 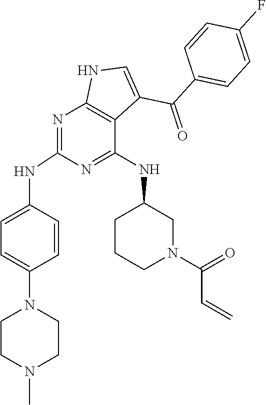 [SMILES: C=CC(=O)N1CCC[C@@H](Nc2nc(Nc3ccc(N4CCN(C)CC4)cc3)nc3[nH]cc(C(=O)c4ccc(F)cc4)c23)C1] | ¹H NMR (600 MHz, DMSO-d₆) δ 12.18 (s, 1H), 8.82 (d, J = 6.8 Hz, 1H), 8.72 (s, 1H), 8.01-7.68 (m, 2H), 7.63 (d, J = 8.3 Hz, 2H), 7.50-7.43 (m, 1H), 7.40-7.28 (m, 2H), 6.90-6.51 (m, 3H), 6.12-5.92 (m, 1H), 5.71-5.39 (m, 1H), 4.32-4.18 (m, 1H), 3.88-3.71 (m, 1H), 3.68-3.40 (m, 2H), 3.33-3.30 (m, 1H), 3.08-2.95 (m, 4H), 2.48-2.44 (m, 4H), 2.23 (s, 3H), 2.11- 2.03 (m, 1H), 1.92-1.80 (m, 1H), 1.79- 1.63 (m, 1H), 1.61-1.50 (m, 1H). | 583.32 |
| 25 | 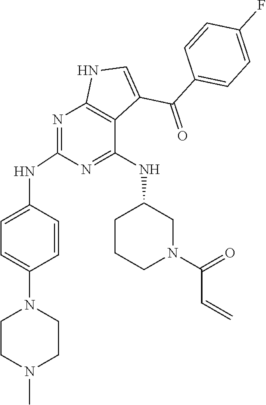 [SMILES: C=CC(=O)N1CCC[C@H](Nc2nc(Nc3ccc(N4CCN(C)CC4)cc3)nc3[nH]cc(C(=O)c4ccc(F)cc4)c23)C1] | ¹H NMR (600 MHz, DMSO-d₆) δ 12.19 (s, 1H), 8.82 (d, J = 7.0 Hz, 1H), 8.72 (s, 1H), 7.88-7.75 (m, 2H), 7.63 (d, J = 8.1 Hz, 2H), 7.46 (s, 1H), 7.38-7.28 (m, 2H), 6.95-6.47 (m, 3H), 6.13-5.90 (m, 1H), 5.70-5.39 (m, 1H), 4.33-4.17 (m, 1H), 3.89-3.70 (m, 1H), 3.66-3.40 (m, 3H), 3.09-2.96 (m, 4H), 2.49-2.45 (m, 4H), 2.25 (s, 3H), 2.10-2.04 (m, 1H), 1.90-1.80 (m, 1H), 1.79-1.64 (m, 1H), 1.61-1.51 (m, 1H). | 583.37 |
| 26 | 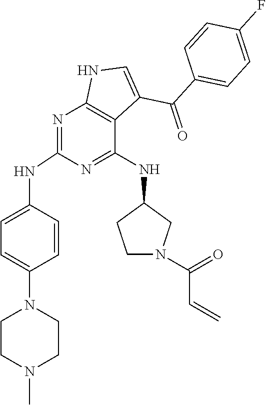 [SMILES: C=CC(=O)N1CC[C@@H](Nc2nc(Nc3ccc(N4CCN(C)CC4)cc3)nc3[nH]cc(C(=O)c4ccc(F)cc4)c23)C1] | ¹H NMR (600 MHz, DMSO-d₆) δ 12.23 (s, 1H), 8.96-8.88 (m, 1H), 8.79 (s, 1H), 7.90-7.79 (m, 2H), 7.71-7.62 (m, 2H), 7.51-7.44 (m, 1H), 7.35 (t, J = 8.6 Hz, 2H), 6.95-6.82 (m, 2H), 6.69-6.55 (m, 1H), 6.19-6.12 (m, 1H), 5.71-5.63 (m, 1H), 4.74-4.60 (m, 1H), 4.11-3.74 (m, 2H), 3.67-3.42 (m, 3H), 3.33-3.32 (m, 1H), 3.13-2.97 (m, 4H), 2.53-2.52 (m, 4H), 2.39-2.21 (m, 4H), 2.13-1.97 (m, 1H). | 569.29 |
| 27 | 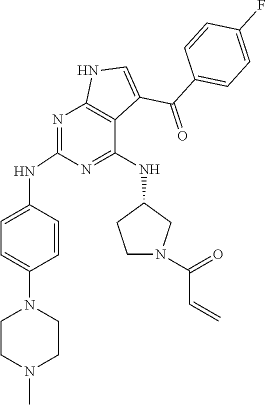 [SMILES: C=CC(=O)N1CC[C@H](Nc2nc(Nc3ccc(N4CCN(C)CC4)cc3)nc3[nH]cc(C(=O)c4ccc(F)cc4)c23)C1] | ¹H NMR (600 MHz, DMSO-d₆) δ 12.23 (s, 1H), 8.91 (dd, J = 15.1, 6.3 Hz, 1H), 8.79 (s, 1H), 7.86-7.78 (m, 2H), 7.66 (dd, J = 11.9, 9.0 Hz, 2H), 7.48 (s, 1H), 7.35 (t, J = 8.6 Hz, 2H), 6.86 (dd, J = 9.2, 2.6 Hz, 2H), 6.70-6.53 (m, 1H), 6.15 (ddd, J = 16.8, 5.3, 2.4 Hz, 1H), 5.68 (ddd, J = 19.4, 10.3, 2.3 Hz, 1H), 4.75-4.59 (m, 1H), 4.11-3.81 (m, 1H), 3.80-3.73 (m, 1H), 3.67-3.46 (m, 3H), 3.33-3.32 (m, 1H), 3.10-3.00 (m, 4H), 2.53-2.51 (m, 4H), 2.38-2.21 (m, 4H), 2.14-1.93 (m, 1H). | 569.32 |
| 28 | 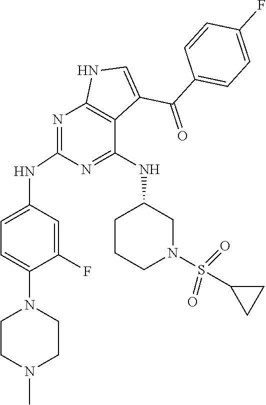 [SMILES: CN1CCN(c2ccc(Nc3nc(N[C@H]4CCCN(S(=O)(=O)C5CC5)C4)c4c(C(=O)c5ccc(F)cc5)c[nH]c4n3)cc2F)CC1] | ¹H NMR (600 MHz, DMSO-d₆) δ 12.31 (s, 1H), 9.07 (s, 1H), 8.92 (d, J = 7.8 Hz, 1H), 7.94-7.79 (m, 3H), 7.53 (s, 1H), 7.41 (d, J = 8.0 Hz, 1H), 7.36 (t, J = 8.7 Hz, 2H), 6.94 (t, J = 9.4 Hz, 1H), 4.46-4.33 (m, 1H), 3.66-3.54 (m, J = 8.9 Hz, 1H), 3.21- 3.10 (m, 2H), 3.07-2.86 (m, 4H), 2.64- 2.57 (m, 2H), 2.56-2.52 (m, 4H), 2.26 (s, 3H), 2.05-1.89 (m, 2H), 1.73-1.59 (m, 2H), 0.98-0.88 (m, 4H). | 651.29 |
| 29 | 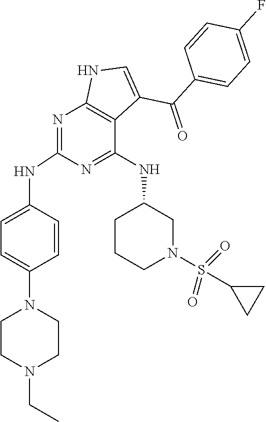 [SMILES: CCN1CCN(c2ccc(Nc3nc(N[C@H]4CCCN(S(=O)(=O)C5CC5)C4)c4c(C(=O)c5ccc(F)cc5)c[nH]c4n3)cc2)CC1] | ¹H NMR (600 MHz, DMSO-d₆) δ 12.21 (s, 1H), 8.85 (d, J = 7.8 Hz, 1H), 8.74 (s, 1H), 7.84 (dd, J = 8.5, 5.6 Hz, 2H), 7.63 (d, J = 8.9 Hz, 2H), 7.47 (s, 1H), 7.36 (t, J = 8.8 Hz, 2H), 6.86 (d, J = 8.9 Hz, 2H), 4.43-4.31 (m, 1H), 3.69-3.59 (m, 1H), 3.18-2.97 (m, 6H), 2.64-2.58 (m, 2H), 2.57-2.52 (m, 4H), 2.46-2.36 (m, 2H), 2.05-1.88 (m, 2H), 1.74-1.58 (m, 2H), 1.05 (t, J = 7.0 Hz, 3H), 0.97-0.87 (m, 4H). | 647.33 |
| 30 | 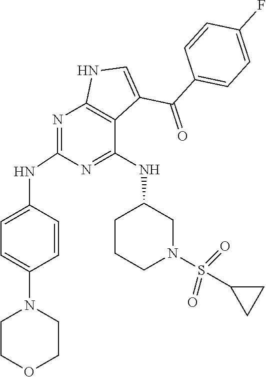 [SMILES: O=C(c1ccc(F)cc1)c1c[nH]c2nc(Nc3ccc(N4CCOCC4)cc3)nc(N[C@H]3CCCN(S(=O)(=O)C4CC4)C3)c12] | ¹H NMR (600 MHz, DMSO-d₆) δ 12.21 (s, 1H), 8.85 (d, J = 7.6 Hz, 1H), 8.76 (s, 1H), 7.98-7.74 (m, 2H), 7.65 (d, J = 8.6 Hz, 2H), 7.48 (s, 1H), 7.36 (t, J = 8.6 Hz, 2H), 6.86 (d, J = 8.6 Hz, 2H), 4.49-4.26 (m, 1H), 3.82-3.68 (m, 4H), 3.68-3.58 (m, 1H), 3.33-3.32 (m, 1H), 3.20-3.06 (m, 2H), 3.06-2.95 (m, 4H), 2.61-2.58 (m, 1H), 2.08-1.86 (m, 2H), 1.75-1.57 (m, 2H), 1.03-0.87 (m, 4H). | 620.28 |
| 31 | 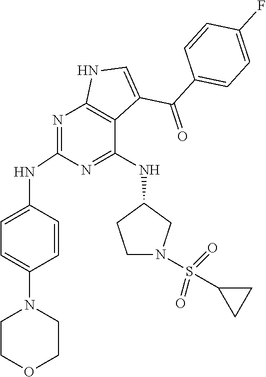 [SMILES: O=C(c1ccc(F)cc1)c1c[nH]c2nc(Nc3ccc(N4CCOCC4)cc3)nc(N[C@H]3CCN(S(=O)(=O)C4CC4)C3)c12] | ¹H NMR (600 MHz, DMSO-d₆) δ 12.24 (s, 1H), 8.94 (d, J = 6.2 Hz, 1H), 8.80 (s, 1H), 7.90-7.77 (m, 2H), 7.67 (dd, J = 9.0, 3.7 Hz, 2H), 7.50 (d, J = 3.0 Hz, 1H), 7.41- 7.31 (m, 2H), 6.88 (d, J = 9.1 Hz, 2H), 4.73-4.63 (m, 1H), 3.81-3.77 (m, 1H), 3.76-3.72 (m, 4H), 3.59-3.47 (m, 2H), 3.33-3.31 (m, 1H), 3.06-2.99 (m, 4H), 2.72-2.65 (m, 1H), 2.43-2.38 (m, 1H), 2.08-2.01 (m, 1H), 0.98-0.89 (m, 4H). | 606.26 |
| 32 | 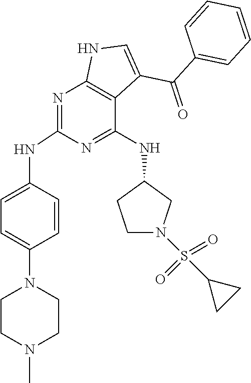 [SMILES: CN1CCN(c2ccc(Nc3nc(N[C@H]4CCN(S(=O)(=O)C5CC5)C4)c4c(C(=O)c5ccccc5)c[nH]c4n3)cc2)CC1] | ¹H NMR (600 MHz, DMSO-d₆) δ 12.22 (s, 1H), 9.00 (d, J = 6.2 Hz, 1H), 8.78 (s, 1H), 7.78-7.73 (m, 2H), 7.67-7.60 (m, 3H), 7.54 (t, J = 7.6 Hz, 2H), 7.45 (s, 1H), 6.87 (d, J = 9.1 Hz, 2H), 4.74-4.63 (m, 1H), 3.83-3.76 (m, 1H), 3.58-3.48 (m, 2H), 3.33-3.31 (m, 1H), 3.14-3.00 (m, 4H), 2.73-2.66 (m, 1H), 2.54-2.51 (m, 4H), 2.44-2.39 (m, 1H), 2.25 (s, 3H), 2.10- 2.02 (m, 1H), 0.97-0.89 (m, 4H). | 601.30 |
| 33 | 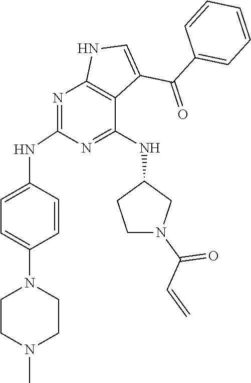 [SMILES: C=CC(=O)N1CC[C@H](Nc2nc(Nc3ccc(N4CCN(C)CC4)cc3)nc3[nH]cc(C(=O)c4ccccc4)c23)C1] | ¹H NMR (600 MHz, DMSO-d₆) δ 12.21 (s, 1H), 8.99 (dd, J = 15.2, 6.1 Hz, 1H), 8.79 (s, 1H), 7.74 (d, J = 7.4 Hz, 2H), 7.70- 7.59 (m, 3H), 7.53 (t, J = 7.4 Hz, 2H), 7.43 (s, 1H), 6.86 (d, J = 6.6 Hz, 2H), 6.62 (ddd, J = 45.6, 16.7, 10.3 Hz, 1H), 6.16 (dd, J = 16.6, 2.5 Hz, 1H), 5.68 (dd, J = 18.6, 10.3 Hz, 1H), 4.79-4.56 (m, 1H), 4.12-3.75 (m, 2H), 3.64-3.44 (m, 2H), 3.14-2.99 (m, 4H), 2.53-2.52 (m, 4H), 2.40-2.17 (m, 4H), 2.15-1.95 (m, 1H). | 551.30 |
| 34 | 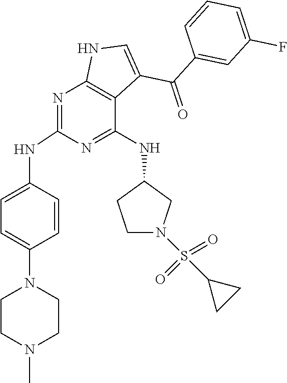 [SMILES: CN1CCN(c2ccc(Nc3nc(N[C@H]4CCN(S(=O)(=O)C5CC5)C4)c4c(C(=O)c5cccc(F)c5)c[nH]c4n3)cc2)CC1] | ¹H NMR (600 MHz, DMSO-d₆) δ 12.28 (s, 1H), 8.91 (d, J = 6.1 Hz, 1H), 8.80 (s, 1H), 7.64 (d, J = 8.9 Hz, 2H), 7.60-7.51 (m, 4H), 7.49-7.44 (m, 1H), 6.87 (d, J = 9.0 Hz, 2H), 4.75-4.61 (m, 1H), 3.84-3.74 (m, 1H), 3.59-3.48 (m, 2H), 3.33-3.31 (m, 1H), 3.12-3.00 (m, 4H), 2.73-2.66 (m, 1H), 2.53-2.52 (m, 4H), 2.44-2.39 (m, 1H), 2.26 (s, 3H), 2.14-1.93 (m, 1H), 0.97- 0.89 (m, 4H). | 619.28 |
| 35 | 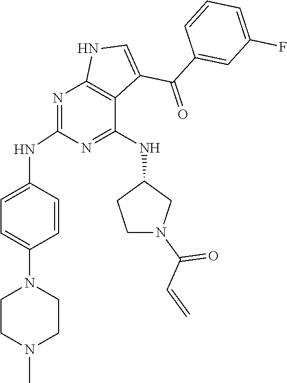 [SMILES: C=CC(=O)N1CC[C@H](Nc2nc(Nc3ccc(N4CCN(C)CC4)cc3)nc3[nH]cc(C(=O)c4cccc(F)c4)c23)C1] | ¹H NMR (600 MHz, DMSO-d₆) δ 12.27 (s, 1H), 8.89 (dd, J = 14.0, 6.3 Hz, 1H), 8.80 (s, 1H), 7.66 (dd, J = 12.0, 9.0 Hz, 2H), 7.60-7.50 (m, 4H), 7.49-7.43 (m, 1H), 6.86 (dd, J = 9.2, 2.7 Hz, 2H), 6.62 (ddd, J = 46.8, 16.8, 10.3 Hz, 1H), 6.15 (ddd, J = 16.8, 5.3, 2.4 Hz, 1H), 5.68 (ddd, J = 19.3, 10.3, 2.4 Hz, 1H), 4.84-4.54 (m, 1H), 4.11-3.73 (m, 2H), 3.71-3.43 (m, 2H), 3.11-2.97 (m, 4H), 2.53-2.52 (m, 4H), 2.39-2.19 (m, 4H), 2.16-1.95 (m, 1H). | 569.30 |
| 36 | 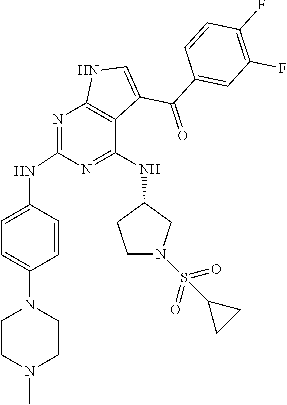 [SMILES: CN1CCN(c2ccc(Nc3nc(N[C@H]4CCN(S(=O)(=O)C5CC5)C4)c4c(C(=O)c5ccc(F)c(F)c5)c[nH]c4n3)cc2)CC1] | ¹H NMR (600 MHz, DMSO-d₆) δ 12.30 (s, 1H), 8.85 (d, J = 6.2 Hz, 1H), 8.80 (s, 1H), 7.86-7.81 (m, 1H), 7.68-7.56 (m, 5H), 6.87 (d, J = 9.1 Hz, 2H), 4.73-4.61 (m, 1H), 3.84-3.75 (m, 1H), 3.57-3.48 (m, 2H), 3.33-3.32 (m, 1H), 3.12-2.98 (m, 4H), 2.73-2.66 (m, 1H), 2.54-2.52 (m, 4H), 2.44-2.39 (m, 1H), 2.25 (s, 3H), 2.07- 2.00 (m, 1H), 0.99-0.87 (m, 4H). | 637.28 |
| 37 | 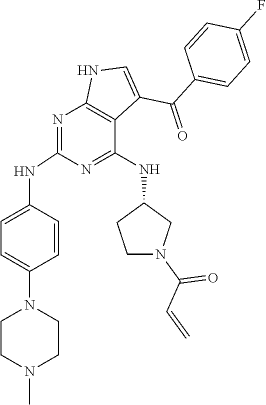 [SMILES: C=CC(=O)N1CC[C@H](Nc2nc(Nc3ccc(N4CCN(C)CC4)cc3)nc3[nH]cc(C(=O)c4ccc(F)cc4)c23)C1] | ¹H NMR (600 MHz, DMSO-d₆) δ 12.29 (s, 1H), 8.97-8.73 (m, 2H), 7.85-7.80 (m, 1H), 7.69-7.54 (m, 5H), 6.86 (dd, J = 9.0, 2.4 Hz, 2H), 6.62 (ddd, J = 47.6, 16.7, 10.3 Hz, 1H), 6.15 (ddd, J = 16.8, 5.4, 2.3 Hz, 1H), 5.68 (ddd, J = 20.0, 10.3, 2.2 Hz, 1H), 4.77-4.59 (m, 1H), 4.10-3.46 (m, 4H), 3.13-3.00 (m, 4H), 2.53-2.52 (m, 4H), 2.38-2.19 (m, 4H), 2.12-1.96 (m, 1H). | 587.26 |
| 38 | 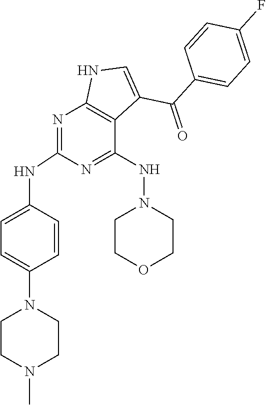 [SMILES: CN1CCN(c2ccc(Nc3nc(NN4CCOCC4)c4c(C(=O)c5ccc(F)cc5)c[nH]c4n3)cc2)CC1] | ¹H NMR (600 MHz, DMSO-d₆) δ 12.24 (s, 1H), 9.59 (s, 1H), 8.87 (s, 1H), 7.85 (dd, J = 8.6, 5.6 Hz, 2H), 7.80 (d, J = 8.4 Hz, 2H), 7.49 (s, 1H), 7.36 (t, J = 8.8 Hz, 2H), 6.90-6.82 (m, 2H), 3.86-3.69 (m, 4H), 3.14-3.01 (m, 4H), 3.01-2.88 (m, 4H), 2.58-2.53 (m, 4H), 2.29 (s, 3H). | 531.27 |
| 39 | 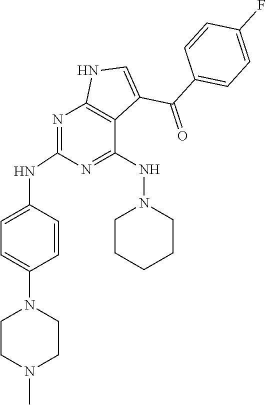 [SMILES: CN1CCN(c2ccc(Nc3nc(NN4CCCCC4)c4c(C(=O)c5ccc(F)cc5)c[nH]c4n3)cc2)CC1] | ¹H NMR (600 MHz, DMSO) δ 12.20 (s, 1H), 9.56 (s, 1H), 8.83 (s, 1H), 7.92-7.83 (m, 2H), 7.79 (d, J = 8.5 Hz, 2H), 7.46 (s, 1H), 7.40-7.30 (m, 2H), 6.84 (d, J = 9.1 Hz, 2H), 3.11-2.99 (m, 4H), 2.97-2.76 (m, 4H), 2.54-2.52 (m, 4H), 2.26 (s, 3H), 1.75- 1.65 (m, 4H), 1.52-1.43 (m, 2H). | 529.23 |
| 40 | 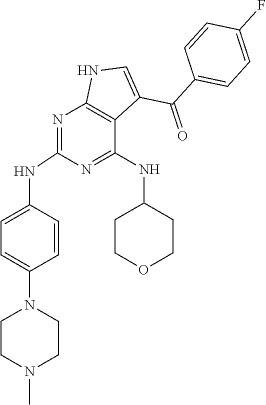 [SMILES: CN1CCN(c2ccc(Nc3nc(NC4CCOCC4)c4c(C(=O)c5ccc(F)cc5)c[nH]c4n3)cc2)CC1] | ¹H NMR (600 MHz, DMSO-d₆) δ 12.16 (s, 1H), 8.77 (d, J = 7.2 Hz, 1H), 8.71 (s, 1H), 7.90-7.77 (m, 2H), 7.69-7.62 (m, 2H), 7.46 (s, 1H), 7.39-7.32 (m, 2H), 6.88- 6.83 (m, 2H), 4.32-4.21 (m, 1H), 3.96- 3.89 (m, 2H), 3.56-3.48 (m, 2H), 3.10- 2.99 (m, 4H), 2.49-2.45 (m, 4H), 2.24 (s, 3H), 2.13-2.00 (m, 2H), 1.59-1.50 (m, 2H). | 530.23 |
| 41 | 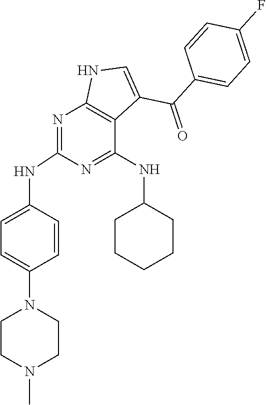 [SMILES: CN1CCN(c2ccc(Nc3nc(NC4CCCCC4)c4c(C(=O)c5ccc(F)cc5)c[nH]c4n3)cc2)CC1] | ¹H NMR (600 MHz, DMSO-d₆) δ 12.12 (s, 1H), 8.74 (d, J = 7.6 Hz, 1H), 8.67 (s, 1H), 7.87-7.81 (m, 2H), 7.71-7.65 (m, 2H), 7.43 (s, 1H), 7.38-7.33 (m, 2H), 6.84 (d, J = 9.1 Hz, 2H), 4.18-4.03 (m, 1H), 3.10-2.98 (m, 4H), 2.49-2.46 (m, 4H), 2.24 (s, 3H), 2.09-1.95 (m, 2H), 1.82-1.73 (m, 2H), 1.63-1.30 (m, 6H). | 528.25 |
| 42 | 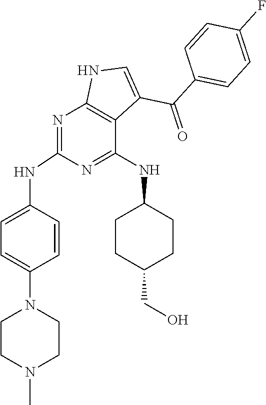 [SMILES: CN1CCN(c2ccc(Nc3nc(N[C@H]4CC[C@H](CO)CC4)c4c(C(=O)c5ccc(F)cc5)c[nH]c4n3)cc2)CC1] | ¹H NMR (600 MHz, DMSO-d₆) δ 12.13 (s, 1H), 8.86-8.49 (m, 2H), 7.88-7.79 (m, 2H), 7.67 (dd, J = 9.8, 2.7 Hz, 2H), 7.43 (s, 1H), 7.39-7.31 (m, 2H), 6.84 (d, J = 9.1 Hz, 2H), 4.47 (t, J = 5.3 Hz, 1H), 4.08- 3.91 (m, 1H), 3.32-3.26 (m, 2H), 3.11- 2.97 (m, 4H), 2.49-2.46 (m, 4H), 2.24 (s, 3H), 2.19-2.10 (m, 2H), 1.88-1.79 (m, 2H), 1.48-1.38 (m, 1H), 1.32-1.24 (m, 2H), 1.14-1.02 (m, 2H). | 558.27 |
| 43 | 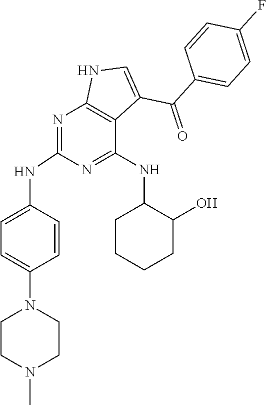 [SMILES: CN1CCN(c2ccc(Nc3nc(NC4CCCCC4O)c4c(C(=O)c5ccc(F)cc5)c[nH]c4n3)cc2)CC1] | ¹H NMR (600 MHz, DMSO-d₆) δ 12.10 (s, 1H), 8.76 (d, J = 7.4 Hz, 1H), 8.65 (s, 1H), 7.88-7.79 (m, 2H), 7.71-7.64 (m, 2H), 7.42 (d, J = 2.4 Hz, 1H), 7.39-7.30 (m, 2H), 6.84 (d, J = 9.1 Hz, 2H), 4.80 (d, J = 4.2 Hz, 1H), 4.03-3.93 (m, 1H), 3.53- 3.42 (m, 1H), 3.12-2.97 (m, 4H), 2.54- 2.51 (m, 4H), 2.24 (s, 3H), 2.21-2.14 (m, 1H), 1.98-1.89 (m, 1H), 1.75-1.62 (m, 2H), 1.40-1.25 (m, 4H). | 544.30 |
| 44 | 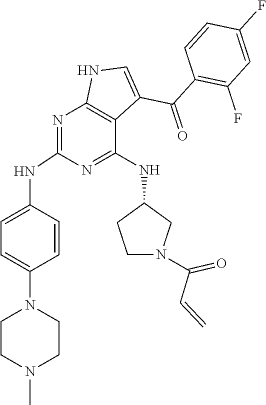 [SMILES: C=CC(=O)N1CC[C@H](Nc2nc(Nc3ccc(N4CCN(C)CC4)cc3)nc3[nH]cc(C(=O)c4ccc(F)cc4F)c23)C1] | ¹H NMR (600 MHz, DMSO-d₆) δ 12.29 (s, 1H), 9.06-8.63 (m, 2H), 7.74-7.37 (m, 5H), 7.21 (t, J = 7.4 Hz, 1H), 6.86 (dd, J = 9.2, 2.8 Hz, 2H), 6.62 (ddd, J = 44.7, 16.8, 10.3 Hz, 1H), 6.15 (ddd, J = 16.8, 4.6, 2.4 Hz, 1H), 5.68 (ddd, J = 18.8, 10.3, 2.3 Hz, 1H), 4.81-4.54 (m, 1H), 4.11-3.74 (m, 2H), 3.65-3.45 (m, 2H), 3.11-2.98 (m, 4H), 2.53-2.52 (m, 4H), 2.38-2.28 (m, 1H), 2.25 (s, 3H), 2.15-1.97 (m, 1H). | 587.27 |
| 45 | 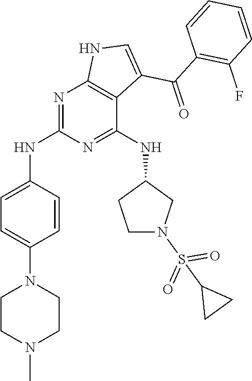 [SMILES: CN1CCN(c2ccc(Nc3nc(N[C@H]4CCN(S(=O)(=O)C5CC5)C4)c4c(C(=O)c5ccccc5F)c[nH]c4n3)cc2)CC1] | ¹H NMR (600 MHz, DMSO-d₆) δ 12.29 (s, 1H), 8.87 (d, J = 6.1 Hz, 1H), 8.81 (s, 1H), 7.67-7.57 (m, 4H), 7.42-7.29 (m, 3H), 6.87 (d, J = 9.1 Hz, 2H), 4.76-4.63 (m, 1H), 3.84-3.72 (m, 1H), 3.61-3.46 (m, 2H), 3.36-3.35 (m, 1H), 3.10-3.02 (m, 4H), 2.70-2.64 (m, 1H), 2.50-2.45 (m, 4H), 2.45-2.37 (m, 1H), 2.24 (s, 3H), 2.10- 2.01 (m, 1H), 0.97-0.87 (m, 4H). | 619.26 |
| 46 | 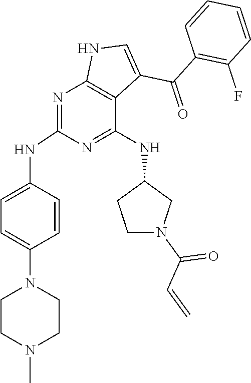 [SMILES: C=CC(=O)N1CC[C@H](Nc2nc(Nc3ccc(N4CCN(C)CC4)cc3)nc3[nH]cc(C(=O)c4ccccc4F)c23)C1] | ¹H NMR (600 MHz, DMSO-d₆) δ 12.27 (s, 1H), 8.98-8.68 (m, 2H), 7.72-7.55 (m, 4H), 7.41-7.28 (m, 3H), 6.86 (dd, J = 9.2, 2.7 Hz, 2H), 6.62 (ddd, J = 43.7, 16.7, 10.3 Hz, 1H), 6.16 (ddd, J = 16.8, 4.6, 2.4 Hz, 1H), 5.68 (ddd, J = 18.2, 10.3, 2.3 Hz, 1H), 4.80-4.58 (m, 1H), 4.14-3.81 (m, 1H), 3.81-3.72 (m, 1H), 3.63-3.47 (m, 2H), 3.10-3.00 (m, 4H), 2.50-2.44 (m, 4H), 2.42-2.29 (m, 1H), 2.25 (s, 3H), 2.16-1.97 (m, 1H). | 569.28 |
| 47 | 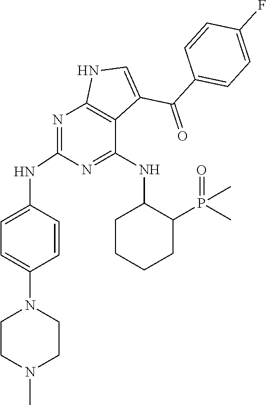 [SMILES: CN1CCN(c2ccc(Nc3nc(NC4CCCCC4P(C)(C)=O)c4c(C(=O)c5ccc(F)cc5)c[nH]c4n3)cc2)CC1] | ¹H NMR (600 MHz, DMSO-d₆) δ 12.41 (s, 1H), 10.32 (s, 1H), 8.77 (s, 1H), 7.95- 7.85 (m, 3H), 7.73-7.63 (m, 3H), 7.57- 7.41 (m, 3H), 7.41-7.34 (m, 2H), 6.71 (d, J = 8.0 Hz, 2H), 3.09-2.92 (m, 4H), 2.55- 2.51 (m, 4H), 2.26 (s, 3H), 1.68 (d, J = 13.4 Hz, 6H). | 598.26 |
| 48 | 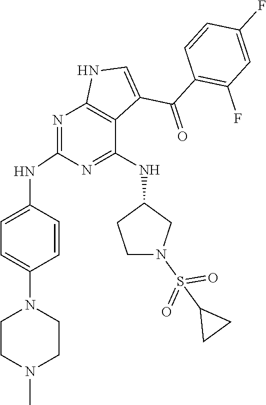 [SMILES: CN1CCN(c2ccc(Nc3nc(N[C@H]4CCN(S(=O)(=O)C5CC5)C4)c4c(C(=O)c5ccc(F)cc5F)c[nH]c4n3)cc2)CC1] | ¹H NMR (600 MHz, DMSO-d₆) δ 12.31 (s, 1H), 8.82 (s, 1H), 8.81 (s, 1H), 7.72- 7.61 (m, 3H), 7.45 (s, 1H), 7.42 (dd, J = 9.8, 2.3 Hz, 1H), 7.25-7.19 (m, J = 8.4, 2.1 Hz, 1H), 6.87 (d, J = 9.1 Hz, 2H), 4.79- 4.64 (m, 1H), 3.85-3.72 (m, 1H), 3.58- 3.47 (m, 2H), 3.33-3.28 (m, 1H), 3.09- 3.03 (m, 4H), 2.70-2.64 (m, 1H), 2.49- 2.46 (m, 4H), 2.44-2.38 (m, 1H), 2.24 (s, 3H), 2.09-2.01 (m, 1H), 0.96-0.89 (m, 4H). | 637.05 |
| 49 | 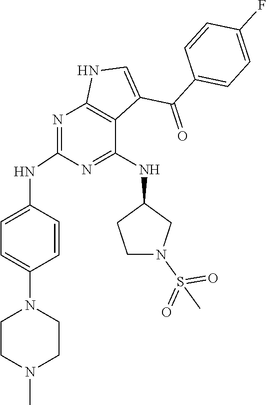 [SMILES: CN1CCN(c2ccc(Nc3nc(N[C@@H]4CCN(S(C)(=O)=O)C4)c4c(C(=O)c5ccc(F)cc5)c[nH]c4n3)cc2)CC1] | ¹H NMR (600 MHz, DMSO-d₆) δ 12.24 (s, 1H), 8.92 (d, J = 6.2 Hz, 1H), 8.78 (s, 1H), 7.89-7.80 (m, 2H), 7.68-7.61 (m, 2H), 7.50 (s, 1H), 7.39-7.33 (m, 2H), 6.87 (d, J = 9.1 Hz, 2H), 4.71-4.60 (m, 1H), 3.79-3.70 (m, 1H), 3.54-3.41 (m, 2H), 3.27-3.20 (m, 1H), 3.11-2.98 (m, 4H), 2.93 (s, 3H), 2.49-2.45 (m, 4H), 2.41-2.35 (m, 1H), 2.24 (s, 3H), 2.09-1.99 (m, 1H). | 593.18 |
| 50 | 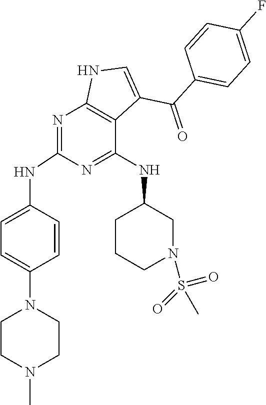 [SMILES: CN1CCN(c2ccc(Nc3nc(N[C@@H]4CCCN(S(C)(=O)=O)C4)c4c(C(=O)c5ccc(F)cc5)c[nH]c4n3)cc2)CC1] | ¹H NMR (600 MHz, DMSO-d₆) δ 12.21 (s, 1H), 8.85 (d, J = 7.9 Hz, 1H), 8.75 (s, 1H), 7.89-7.78 (m, 2H), 7.66-7.59 (m, 2H), 7.47 (s, 1H), 7.35 (dd, J = 12.3, 5.4 Hz, 2H), 6.86 (d, J = 9.1 Hz, 2H), 4.45- 4.32 (m, 1H), 3.61-3.54 (m, 1H), 3.31- 3.27 (m, 1H), 3.10-2.97 (m, 6H), 2.89 (s, 3H), 2.49-2.46 (m, 4H), 2.24 (s, 3H), 2.05- 1.86 (m, 2H), 1.74-1.54 (m, 2H). | 607.22 |
| 51 | 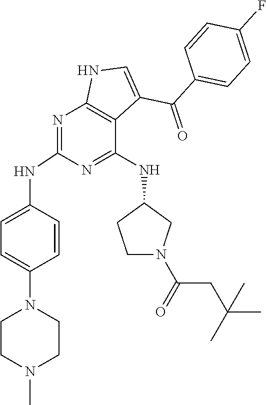 [SMILES: CN1CCN(c2ccc(Nc3nc(N[C@H]4CCN(C(=O)CC(C)(C)C)C4)c4c(C(=O)c5ccc(F)cc5)c[nH]c4n3)cc2)CC1] | ¹H NMR (600 MHz, DMSO-d₆) δ 12.21 (s, 1H), 9.00-8.84 (m, 1H), 8.78 (d, J = 6.1 Hz, 1H), 7.88-7.73 (m, 2H), 7.71-7.61 (m, 2H), 7.48 (s, 1H), 7.41-7.31 (m, 2H), 6.86 (d, J = 7.7 Hz, 2H), 4.75-4.56 (m, 1H), 3.96-3.39 (m, 4H), 3.14-2.98 (m, 4H), 2.53-2.52 (m, 4H), 2.37-2.25 (m, 4H), 2.21-2.10 (m, 2H), 2.08-1.91 (m, 1H), 0.98 (d, J = 16.9 Hz, 9H). | 613.31 |
| 52 | 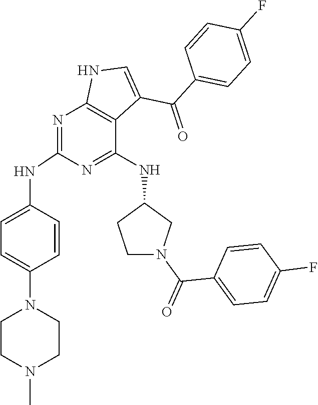 [SMILES: CN1CCN(c2ccc(Nc3nc(N[C@H]4CCN(C(=O)c5ccc(F)cc5)C4)c4c(C(=O)c5ccc(F)cc5)c[nH]c4n3)cc2)CC1] | ¹H NMR (600 MHz, DMSO-d₆) δ 12.23 (s, 1H), 8.95 (dd, J = 39.8, 6.0 Hz, 1H), 8.75 (d, J = 53.9 Hz, 1H), 7.88-7.81 (m, 2H), 7.71-7.61 (m, 3H), 7.55 (d, J = 8.5 Hz, 1H), 7.49 (d, J = 13.7 Hz, 1H), 7.41- 7.33 (m, 2H), 7.32-7.20 (m, 2H), 6.87 (d, J = 8.8 Hz, 1H), 6.78 (d, J = 8.7 Hz, 1H), 4.76-4.58 (m, 1H), 4.00-3.89 (m, 1H), 3.77-3.44 (m, 3H), 3.08-2.99 (m, 4H), 2.48-2.44 (m, 4H), 2.39-2.28 (m, 1H), 2.23 (s, 3H), 2.13-1.97 (m, 1H). | 637.23 |
| 53 | 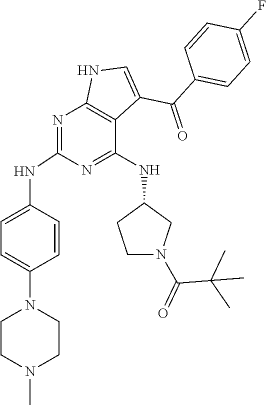 [SMILES: CN1CCN(c2ccc(Nc3nc(N[C@H]4CCN(C(=O)C(C)(C)C)C4)c4c(C(=O)c5ccc(F)cc5)c[nH]c4n3)cc2)CC1] | ¹H NMR (600 MHz, DMSO-d₆) δ 12.21 (s, 1H), 8.90 (d, J = 6.4 Hz, 1H), 8.78 (s, 1H), 7.82 (dd, J = 8.5, 5.6 Hz, 2H), 7.66 (d, J = 8.8 Hz, 2H), 7.48 (s, 1H), 7.35 (t, J = 8.8 Hz, 2H), 6.86 (d, J = 9.0 Hz, 2H), 4.82-4.48 (m, 1H), 4.00-3.44 (m, 4H), 3.15-2.98 (m, 4H), 2.54-2.52 (m, 4H), 2.35-2.18 (m, 4H), 2.10-1.86 (m, 1H), 1.17 (s, 9H). | 599.27 |
| 54 | 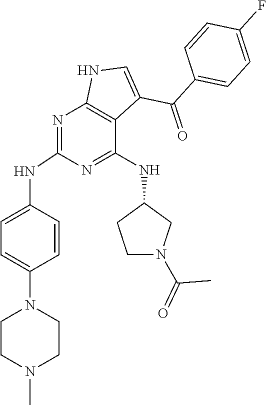 [SMILES: CC(=O)N1CC[C@H](Nc2nc(Nc3ccc(N4CCN(C)CC4)cc3)nc3[nH]cc(C(=O)c4ccc(F)cc4)c23)C1] | ¹H NMR (600 MHz, DMSO-d₆) δ 12.23 (s, 1H), 8.91 (dd, J = 21.6, 6.4 Hz, 1H), 8.77 (s, 1H), 7.87-7.80 (m, 2H), 7.69-7.62 (m, 2H), 7.48 (d, J = 3.2 Hz, 1H), 7.35 (t, J = 8.8 Hz, 2H), 6.86 (dd, J = 9.1, 2.2 Hz, 2H), 4.75-4.57 (m, 1H), 3.96-3.42 (m, 4H), 3.11-3.00 (m, 4H), 2.50-2.46 (m, 4H), 2.40-2.27 (m, 1H), 2.24 (s, 3H), 2.11- 1.93 (m, 4H). | 557.21 |
| 55 | 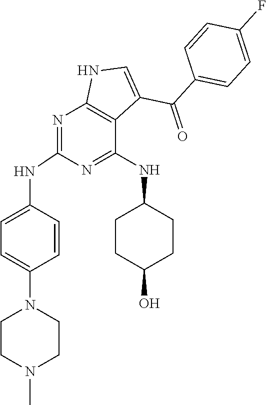 [SMILES: CN1CCN(c2ccc(Nc3nc(N[C@H]4CC[C@@H](O)CC4)c4c(C(=O)c5ccc(F)cc5)c[nH]c4n3)cc2)CC1] | ¹H NMR (600 MHz, DMSO-d₆) δ 12.10 (s, 1H), 8.82 (d, J = 7.5 Hz, 1H), 8.63 (s, 1H), 7.89-7.81 (m, 2H), 7.71-7.62 (m, 2H), 7.42 (s, 1H), 7.38-7.32 (m, 2H), 6.83 (d, J = 9.1 Hz, 2H), 4.52 (d, J = 3.4 Hz, 1H), 4.24-4.13 (m, 1H), 3.73-3.65 (m, 1H), 3.07-3.01 (m, 4H), 2.47-2.43 (m, 4H), 2.22 (s, 3H), 1.83-1.70 (m, 4H), 1.70- 1.62 (m, 4H). | 544.23 |
| 56 | 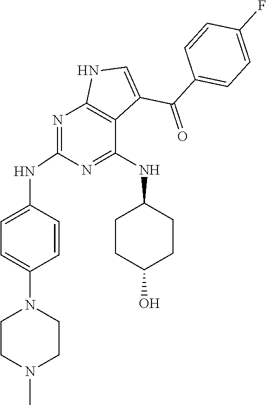 [SMILES: CN1CCN(c2ccc(Nc3nc(N[C@H]4CC[C@H](O)CC4)c4c(C(=O)c5ccc(F)cc5)c[nH]c4n3)cc2)CC1] | ¹H NMR (600 MHz, DMSO-d₆) δ 12.10 (s, 1H), 8.69-8.60 (m, J = 8.5 Hz, 2H), 7.90-7.77 (m, 2H), 7.71-7.62 (m, 2H), 7.42 (s, 1H), 7.39-7.30 (m, 2H), 6.84 (d, J = 9.1 Hz, 2H), 4.60 (d, J = 4.4 Hz, 1H), 4.08-3.95 (m, 1H), 3.60-3.51 (m, 1H), 3.09-3.00 (m, 4H), 2.49-2.41 (m, 4H), 2.23 (s, 3H), 2.15-2.07 (m, 2H), 1.95-1.87 (m, 2H), 1.40-1.31 (m, 4H). | 544.24 |
| 57 | 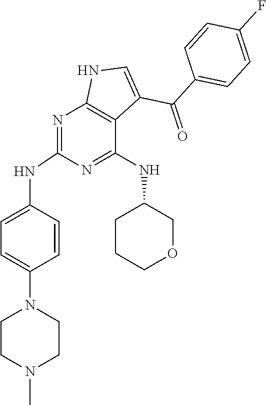 [SMILES: CN1CCN(c2ccc(Nc3nc(N[C@H]4CCCOC4)c4c(C(=O)c5ccc(F)cc5)c[nH]c4n3)cc2)CC1] | ¹H NMR (600 MHz, DMSO-d₆) δ 12.18 (s, 1H), 8.81 (d, J = 7.7 Hz, 1H), 8.72 (s, 1H), 7.88-7.81 (m, 2H), 7.70-7.60 (m, 2H), 7.45 (d, J = 2.1 Hz, 1H), 7.35 (t, J = 8.8 Hz, 2H), 6.84 (d, J = 9.1 Hz, 2H), 4.28- 4.22 (m, 1H), 3.98-3.90 (m, 1H), 3.73- 3.66 (m, 1H), 3.60-3.52 (m, 1H), 3.42- 3.39 (m, 1H), 3.08-3.02 (m, 4H), 2.54- 2.52 (m, 4H), 2.25 (s, 3H), 2.09-2.02 (m, 1H), 1.86-1.79 (m, 1H), 1.73-1.57 (m, 2H). | 530.23 |
| 58 | 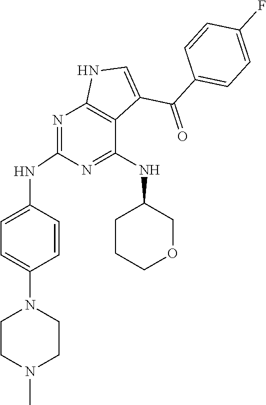 [SMILES: CN1CCN(c2ccc(Nc3nc(N[C@@H]4CCCOC4)c4c(C(=O)c5ccc(F)cc5)c[nH]c4n3)cc2)CC1] | ¹H NMR (600 MHz, DMSO-d₆) δ 12.20 (s, 1H), 8.83 (d, J = 7.7 Hz, 1H), 8.81 (s, 1H), 7.89-7.80 (m, 2H), 7.71 (d, J = 9.1 Hz, 2H), 7.47 (d, J = 2.8 Hz, 1H), 7.41- 7.32 (m, 2H), 6.91 (d, J = 9.1 Hz, 2H), 4.29-4.21 (m, 1H), 3.98-3.90 (m, 1H), 3.74-3.65 (m, 1H), 3.60-3.53 (m, 1H), 3.42-3.39 (m, 1H), 3.30-3.17 (m, 4H), 2.81 (s, 3H), 2.54-2.52 (m, 4H), 2.10-2.02 (m, 1H), 1.88-1.79 (m, 1H), 1.74-1.56 (m, 2H). | 530.22 |
| 59 | 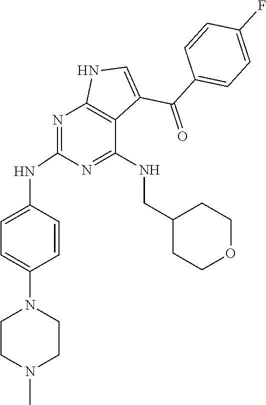 [SMILES: CN1CCN(c2ccc(Nc3nc(NCC4CCOCC4)c4c(C(=O)c5ccc(F)cc5)c[nH]c4n3)cc2)CC1] | ¹H NMR (600 MHz, DMSO-d₆) δ 12.17 (s, 1H), 8.78 (t, J = 5.6 Hz, 1H), 8.70 (s, 1H), 7.86-7.80 (m, 2H), 7.67 (d, J = 9.1 Hz, 2H), 7.45 (s, 1H), 7.39-7.32 (m, 2H), 6.85 (d, J = 9.1 Hz, 2H), 3.94-3.85 (m, 2H), 3.51-3.43 (m, 2H), 3.32-3.28 (m, 2H), 3.12-2.99 (m, 4H), 2.55-2.51 (m, 4H), 2.25 (s, 3H), 1.97-1.84 (m, 1H), 1.74- 1.67 (m, 2H), 1.40-1.28 (m, 2H). | 544.24 |
| 60 | 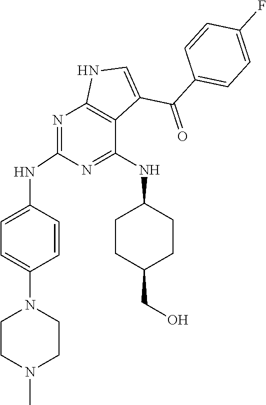 [SMILES: CN1CCN(c2ccc(Nc3nc(N[C@H]4CC[C@@H](CO)CC4)c4c(C(=O)c5ccc(F)cc5)c[nH]c4n3)cc2)CC1] | ¹H NMR (600 MHz, DMSO-d₆) δ 12.13 (s, 1H), 8.96 (d, J = 7.7 Hz, 1H), 8.66 (s, 1H), 7.89-7.80 (m, 2H), 7.66 (d, J = 9.1 Hz, 2H), 7.43 (d, J = 2.2 Hz, 1H), 7.35 (t, J = 8.8 Hz, 2H), 6.84 (d, J = 9.1 Hz, 2H), 4.47 (t, J = 5.3 Hz, 1H), 4.45-4.38 (m, 1H), 3.31-3.27 (m, 2H), 3.11-2.99 (m, 4H), 2.54-2.51 (m, 4H), 2.24 (s, 3H), 1.89-1.80 (m, 2H), 1.71-1.62 (m, 4H), 1.58-1.48 (m, 1H), 1.43-1.33 (m, 2H). | 558.27 |
| 61 | 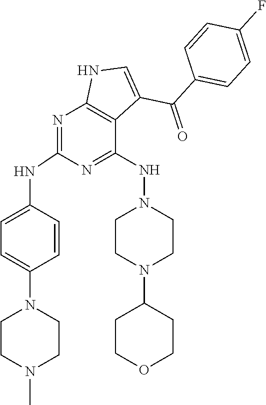 [SMILES: CN1CCN(c2ccc(Nc3nc(NN4CCN(C5CCOCC5)CC4)c4c(C(=O)c5ccc(F)cc5)c[nH]c4n3)cc2)CC1] | ¹H NMR (600 MHz, DMSO-d₆) δ 12.15 (s, 1H), 8.74 (d, J = 5.7 Hz, 1H), 8.68 (s, 1H), 7.94-7.75 (m, 2H), 7.65 (d, J = 8.4 Hz, 2H), 7.44 (s, 1H), 7.36 (t, J = 8.4 Hz, 2H), 6.85 (d, J = 8.5 Hz, 2H), 4.14-4.04 (m, 1H), 3.97-3.84 (m, 2H), 3.30-3.27 (m, 2H), 3.12-2.99 (m, 4H), 2.98-2.85 (m, 2H), 2.55-2.52 (m, 6H), 2.43-2.41 (m, 1H), 2.24 (s, 3H), 2.12-2.01 (m, 2H), 1.78- 1.67 (m, 2H), 1.60-1.42 (m, 4H). | 613.31 |
| 62 | 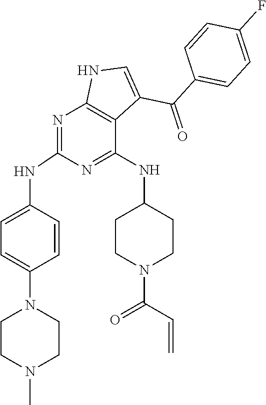 [SMILES: C=CC(=O)N1CCC(Nc2nc(Nc3ccc(N4CCN(C)CC4)cc3)nc3[nH]cc(C(=O)c4ccc(F)cc4)c23)CC1] | ¹H NMR (600 MHz, DMSO-d₆) δ 12.20 (s, 1H), 8.91-8.76 (m, 2H), 7.88-7.79 (m, 2H), 7.72 (d, J = 9.1 Hz, 2H), 7.48 (d, J = 2.9 Hz, 1H), 7.39-7.33 (m, 2H), 6.93 (d, J = 9.1 Hz, 2H), 6.87 (dd, J = 16.7, 10.5 Hz, 1H), 6.13 (dd, J = 16.7, 2.4 Hz, 1H), 5.69 (dd, J = 10.5, 2.4 Hz, 1H), 4.37-4.27 (m, 1H), 4.27-4.17 (m, 1H), 4.08-3.96 (m, 1H), 3.33-3.18 (m, 4H), 3.18-3.08 (m, 2H), 2.82 (s, 3H), 2.53-2.52 (m, 4H), 2.17- 2.06 (m, 2H), 1.59-1.40 (m, 2H). | 583.27 |
| 63 | 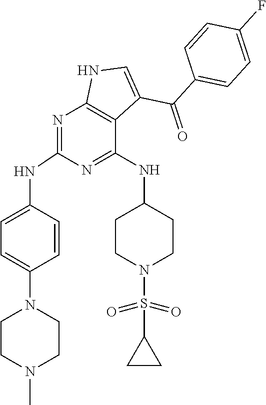 [SMILES: CN1CCN(c2ccc(Nc3nc(NC4CCN(S(=O)(=O)C5CC5)CC4)c4c(C(=O)c5ccc(F)cc5)c[nH]c4n3)cc2)CC1] | ¹H NMR (600 MHz, DMSO-d₆) δ 12.19 (s, 1H), 8.81 (d, J = 7.1 Hz, 1H), 8.72 (s, 1H), 7.89-7.79 (m, 2H), 7.65 (d, J = 9.0 Hz, 2H), 7.47 (s, 1H), 7.35 (t, J = 8.8 Hz, 2H), 6.86 (d, J = 9.1 Hz, 2H), 4.26-4.13 (m, 1H), 3.68-3.57 (m, 2H), 3.18-3.11 (m, 2H), 3.09-3.01 (m, 4H), 2.72-2.65 (m, 1H), 2.54-2.52 (m, 4H), 2.24 (s, 3H), 2.19- 2.13 (m, 2H), 1.69-1.58 (m, 2H), 1.05- 0.95 (m, 4H). | 633.25 |
| 64 | 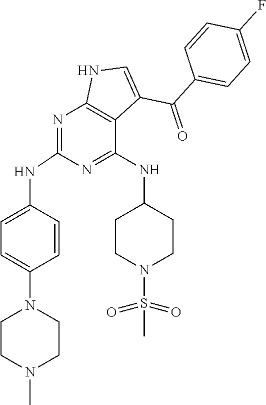 [SMILES: CN1CCN(c2ccc(Nc3nc(NC4CCN(S(C)(=O)=O)CC4)c4c(C(=O)c5ccc(F)cc5)c[nH]c4n3)cc2)CC1] | ¹H NMR (600 MHz, DMSO-d₆) δ 12.19 (s, 1H), 8.81 (d, J = 7.1 Hz, 1H), 8.72 (s, 1H), 7.84 (dd, J = 8.5, 5.6 Hz, 2H), 7.64 (d, J = 9.0 Hz, 2H), 7.47 (s, 1H), 7.36 (t, J = 8.8 Hz, 2H), 6.92-6.83 (m, 2H), 4.24- 4.14 (m, 1H), 3.61-3.52 (m, 2H), 3.10- 2.99 (m, 6H), 2.95 (s, 3H), 2.55-2.51 (m, 4H), 2.25 (s, 3H), 2.20-2.12 (m, 2H), 1.70- 1.58 (m, 2H). | 607.25 |
| 65 | 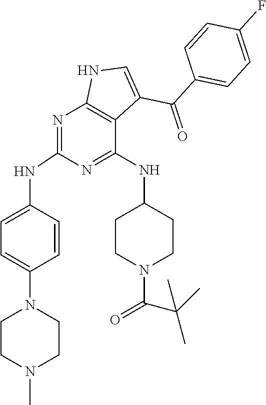 [SMILES: CN1CCN(c2ccc(Nc3nc(NC4CCN(C(=O)C(C)(C)C)CC4)c4c(C(=O)c5ccc(F)cc5)c[nH]c4n3)cc2)CC1] | ¹H NMR (600 MHz, DMSO-d₆) δ 12.17 (s, 1H), 8.80 (d, J = 7.3 Hz, 1H), 8.73 (s, 1H), 7.83 (dd, J = 8.4, 5.7 Hz, 2H), 7.66 (d, J = 8.9 Hz, 2H), 7.46 (s, 1H), 7.35 (t, J = 8.8 Hz, 2H), 6.87 (d, J = 9.0 Hz, 2H), 4.38-4.26 (m, 1H), 4.15-4.06 (m, 2H), 3.26-3.15 (m, 2H), 3.11-2.99 (m, 4H), 2.58-2.46 (m, 4H), 2.24 (s, 3H), 2.19-2.01 (m, 2H), 1.53-1.29 (m, 2H), 1.23 (s, 9H). | 613.32 |
| 66 | 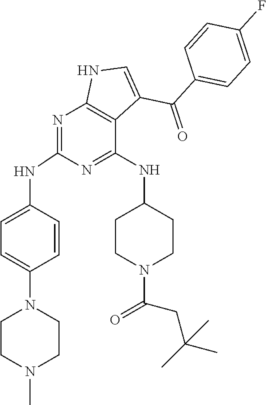 [SMILES: CN1CCN(c2ccc(Nc3nc(NC4CCN(C(=O)CC(C)(C)C)CC4)c4c(C(=O)c5ccc(F)cc5)c[nH]c4n3)cc2)CC1] | ¹H NMR (600 MHz, DMSO-d₆) δ 12.20 (s, 1H), 8.86-8.77 (m, 2H), 7.84 (dd, J = 8.6, 5.6 Hz, 2H), 7.72 (d, J = 9.0 Hz, 2H), 7.48 (d, J = 2.6 Hz, 1H), 7.36 (t, J = 8.8 Hz, 2H), 6.93 (d, J = 9.1 Hz, 2H), 4.33- 4.21 (m, 2H), 4.00-3.92 (m, 1H), 3.28- 2.99 (m, 6H), 2.80 (s, 3H), 2.55-2.51 (m, 4H), 2.28 (d, J = 1.7 Hz, 2H), 2.15-2.03 (m, 2H), 1.55-1.34 (m, 2H), 1.01 (s, 9H) | 627.34 |
| 67 | 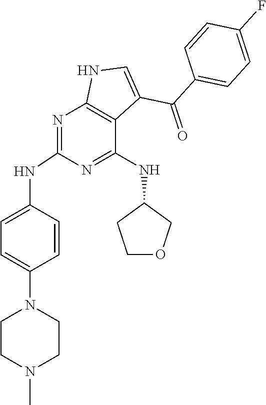 [SMILES: CN1CCN(c2ccc(Nc3nc(N[C@H]4CCOC4)c4c(C(=O)c5ccc(F)cc5)c[nH]c4n3)cc2)CC1] | ¹H NMR (600 MHz, DMSO-d₆) δ 12.20 (s, 1H), 8.90 (d, J = 6.5 Hz, 1H), 8.74 (s, 1H), 7.89-7.81 (m, 2H), 7.66 (d, J = 9.0 Hz, 2H), 7.47 (s, 1H), 7.35 (t, J = 8.8 Hz, 2H), 6.86 (d, J = 9.1 Hz, 2H), 4.73-4.65 (m, 1H), 4.02-3.97 (m, 1H), 3.96-3.89 (m, 1H), 3.85-3.78 (m, 1H), 3.70-3.63 (m, 1H), 3.11-3.00 (m, 4H), 2.53-2.51 (m, 4H), 2.38-2.30 (m, 1H), 2.25 (s, 3H), 1.95- 1.85 (m, 1H). | 516.23 |
| 68 | 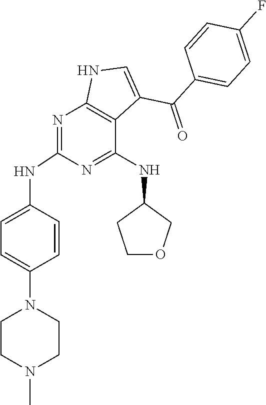 [SMILES: CN1CCN(c2ccc(Nc3nc(N[C@@H]4CCOC4)c4c(C(=O)c5ccc(F)cc5)c[nH]c4n3)cc2)CC1] | ¹H NMR (600 MHz, DMSO-d₆) δ 12.20 (s, 1H), 8.90 (d, J = 6.5 Hz, 1H), 8.74 (s, 1H), 7.88-7.79 (m, 2H), 7.66 (d, J = 9.0 Hz, 2H), 7.47 (s, 1H), 7.35 (t, J = 8.8 Hz, 2H), 6.86 (d, J = 9.1 Hz, 2H), 4.73-4.64 (m, 1H), 4.02-3.96 (m, 1H), 3.96-3.88 (m, 1H), 3.85-3.76 (m, 1H), 3.70-3.62 (m, 1H), 3.12-3.00 (m, 4H), 2.54-2.51 (m, 4H), 2.38-2.31 (m, 1H), 2.25 (s, 3H), 1.95- 1.86 (m, 1H). | 516.23 |
| 69 | 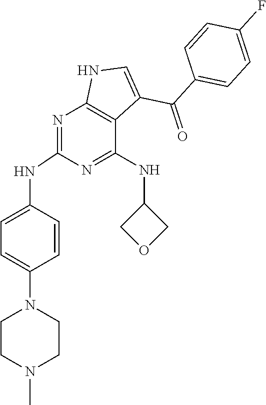 [SMILES: CN1CCN(c2ccc(Nc3nc(NC4COC4)c4c(C(=O)c5ccc(F)cc5)c[nH]c4n3)cc2)CC1] | ¹H NMR (600 MHz, DMSO-d₆) δ 12.88 (s, 1H), 9.34 (s, 1H), 9.11 (s, 1H), 7.97- 7.91 (m, 2H), 7.85 (s, 1H), 7.44-7.36 (m, 4H), 7.01 (d, J = 9.0 Hz, 2H), 4.56-4.48 (m, 2H), 4.44-4.38 (m, 1H), 3.78-3.62 (m, 2H), 3.30-3.09 (m, 4H), 2.76 (s, 3H), 2.54- 2.51 (m, 4H). | 502.21 |
| 70 | 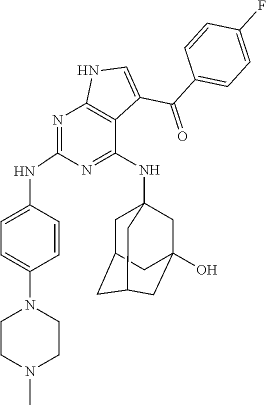 [SMILES: CN1CCN(c2ccc(Nc3nc(NC45CC6CC(CC(O)(C6)C4)C5)c4c(C(=O)c5ccc(F)cc5)c[nH]c4n3)cc2)CC1] | ¹H NMR (600 MHz, DMSO-d₆) δ 12.07 (s, 1H), 8.83 (s, 1H), 8.49 (s, 1H), 7.84- 7.78 (m, 2H), 7.57 (d, J = 8.9 Hz, 2H), 7.38 (s, 1H), 7.36-7.32 (m, 2H), 6.86 (d, J = 9.1 Hz, 2H), 4.52 (s, 1H), 3.14-2.98 (m, 4H), 2.53-2.52 (m, 4H), 2.26 (s, 3H), 2.23- 1.98 (m, 9H), 1.68-1.46 (m, 6H). (84-2) | 596.29 |
| 71 | 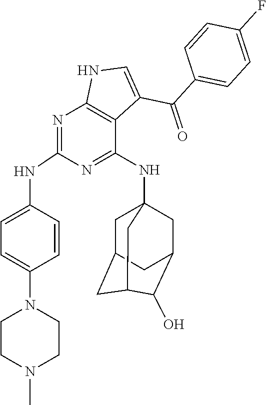 [SMILES: CN1CCN(c2ccc(Nc3nc(NC45CC6CC(C4)C(O)C(C6)C5)c4c(C(=O)c5ccc(F)cc5)c[nH]c4n3)cc2)CC1] | ¹H NMR (600 MHz, DMSO-d₆) δ 12.13 (s, 1H), 9.08 (d, J = 7.8 Hz, 1H), 8.69 (s, 1H), 7.88-7.82 (m, 2H), 7.67 (d, J = 9.1 Hz, 2H), 7.45 (d, J = 2.2 Hz, 1H), 7.35 (t, J = 8.8 Hz, 2H), 6.85 (d, J = 9.1 Hz, 2H), 4.55 (s, 1H), 4.37-4.27 (m, 1H), 3.13-3.01 (m, 4H), 2.53-2.51 (m, 4H), 2.32 (s, 3H), 2.23-2.17 (m, 2H), 2.14-2.09 (m, 1H), 2.04-1.98 (m, 2H), 1.87-1.81 (m, 2H), 1.75-1.70 (m, 2H), 1.69-1.66 (m, 2H), 1.52-1.43 (m, 2H). | 596.30 |
| 72 | 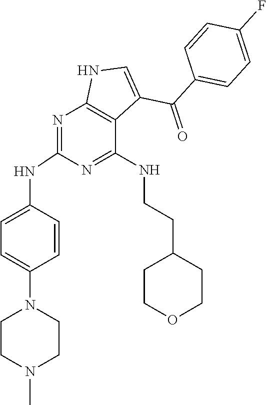 [SMILES: CN1CCN(c2ccc(Nc3nc(NCCC4CCOCC4)c4c(C(=O)c5ccc(F)cc5)c[nH]c4n3)cc2)CC1] | ¹H NMR (600 MHz, DMSO-d₆) δ 12.15 (s, 1H), 8.76-8.59 (m, 2H), 7.87-7.80 (m, 2H), 7.70-7.62 (m, 2H), 7.44 (s, 1H), 7.35 (t, J = 8.8 Hz, 2H), 6.84 (d, J = 9.1 Hz, 2H), 3.88-3.80 (m, 2H), 3.63-3.53 (m, 2H), 3.32-3.25 (m, 2H), 3.09-3.01 (m, 4H), 2.53-2.51 (m, 4H), 2.24 (s, 3H), 1.71- 1.58 (m, 5H), 1.25-1.22 (m, 2H). | 558.27 |
| 73 | 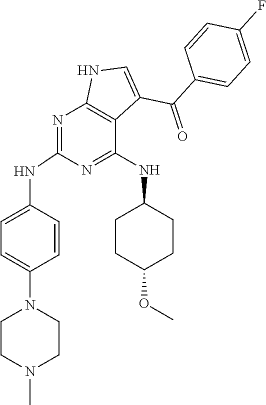 [SMILES: CO[C@H]1CC[C@H](Nc2nc(Nc3ccc(N4CCN(C)CC4)cc3)nc3[nH]cc(C(=O)c4ccc(F)cc4)c23)CC1] | ¹H NMR (600 MHz, DMSO-d₆) δ 12.14 (s, 1H), 8.70 (d, J = 7.4 Hz, 1H), 8.69 (s, 1H), 7.87-7.80 (m, 2H), 7.67 (d, J = 9.0 Hz, 2H), 7.44 (s, 1H), 7.35 (t, J = 8.8 Hz, 2H), 6.84 (d, J = 9.1 Hz, 2H), 4.09-4.01 (m, 1H), 3.27 (s, 3H), 3.27-3.23 (m, 1H), 3.09-3.00 (m, 4H), 2.49-2.45 (m, 4H), 2.24 (s, 3H), 2.16-2.02 (m, 4H), 1.42-1.31 (m, 4H). | 558.27 |
| 74 | 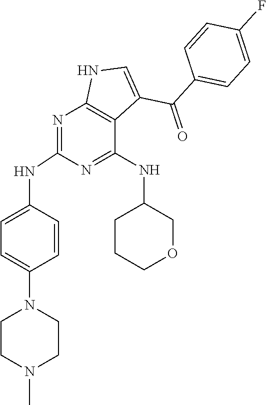 [SMILES: CN1CCN(c2ccc(Nc3nc(NC4CCCOC4)c4c(C(=O)c5ccc(F)cc5)c[nH]c4n3)cc2)CC1] | ¹H NMR (600 MHz, DMSO-d₆) δ 12.17 (s, 1H), 8.82 (d, J = 7.7 Hz, 1H), 8.78 (s, 1H), 7.92-7.78 (m, 2H), 7.74-7.68 (m, 2H), 7.46 (s, J = 2.8 Hz, 1H), 7.39-7.33 (m, 2H), 6.92 (d, J = 8.8 Hz, 2H), 4.32- 4.20 (m, 1H), 3.99-3.90 (m, 1H), 3.73- 3.66 (m, 1H), 3.58-3.54 (m, 1H), 3.42- 3.39 (m, 1H), 3.32-3.28 (m, 4H), 2.82 (s, 3H), 2.52-2.52 (m, 4H), 2.13-1.94 (m, 1H), 1.91-1.76 (m, 1H), 1.73-1.56 (m, 2H). | 530.26 |
| 75 | 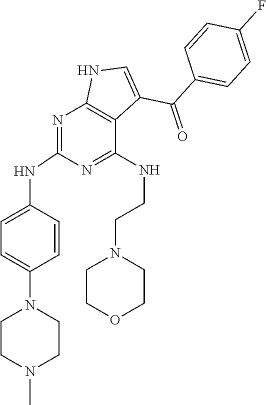 [SMILES: CN1CCN(c2ccc(Nc3nc(NCCN4CCOCC4)c4c(C(=O)c5ccc(F)cc5)c[nH]c4n3)cc2)CC1] | ¹H NMR (600 MHz, DMSO-d₆) δ 12.11 (s, 1H), 8.76 (t, J = 5.3 Hz, 1H), 8.65 (s, 1H), 7.89-7.79 (m, 2H), 7.72-7.63 (m, 2H), 7.43 (s, 1H), 7.35 (t, J = 8.8 Hz, 2H), 6.85 (d, J = 9.0 Hz, 2H), 3.71-3.64 (m, 2H), 3.64-3.58 (m, 4H), 3.12-3.02 (m, 4H), 2.63-2.53 (m, 6H), 2.49-2.42 (m, 4H), 2.30 (s, 3H). | 559.29 |
| 76 | 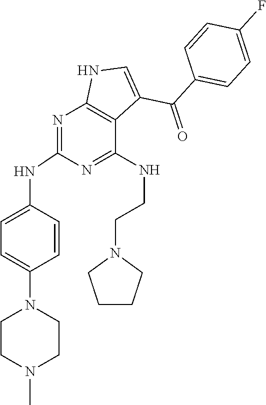 [SMILES: CN1CCN(c2ccc(Nc3nc(NCCN4CCCC4)c4c(C(=O)c5ccc(F)cc5)c[nH]c4n3)cc2)CC1] | ¹H NMR (600 MHz, DMSO-d₆) δ 12.30 (s, 1H), 8.88-8.71 (m, 2H), 7.92-7.77 (m, 2H), 7.66 (d, J = 8.8 Hz, 2H), 7.55 (s, 1H), 7.38 (t, J = 8.8 Hz, 2H), 6.95 (d, J = 8.9 Hz, 2H), 3.92-3.89 (m, 2H), 3.73-3.61 (m, 4H), 3.56-3.51 (m, 2H), 3.51-3.45 (m, 2H), 3.22-3.02 (m, 4H), 2.95-2.83 (m, 5H), 2.07-1.77 (m, 4H). | 543.30 |
| 77 | 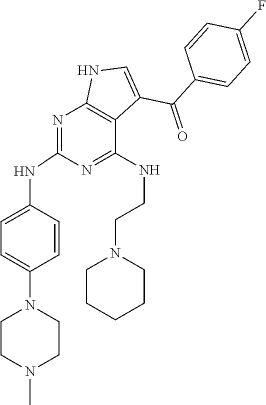 [SMILES: CN1CCN(c2ccc(Nc3nc(NCCN4CCCCC4)c4c(C(=O)c5ccc(F)cc5)c[nH]c4n3)cc2)CC1] | ND | 557.31 |
| 78 | 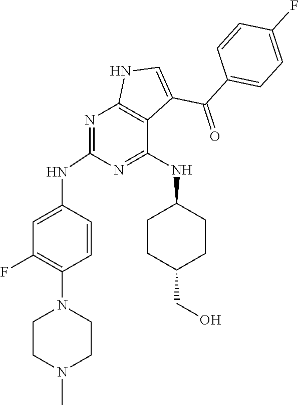 [SMILES: CN1CCN(c2ccc(Nc3nc(N[C@H]4CC[C@H](CO)CC4)c4c(C(=O)c5ccc(F)cc5)c[nH]c4n3)cc2F)CC1] | ¹H NMR (600 MHz, DMSO-d₆) δ 12.18 (s, 1H), 8.97 (s, 1H), 8.71 (d, J = 7.5 Hz, 1H), 8.00 (dd, J = 16.0, 2.3 Hz, 1H), 7.88- 7.79 (m, 2H), 7.47 (d, J = 2.5 Hz, 1H), 7.35 (t, J = 8.8 Hz, 2H), 7.31 (dd, J = 8.7, 1.8 Hz, 1H), 7.02-6.83 (m, 1H), 4.45 (t, J = 5.3 Hz, 1H), 4.03-3.94 (m, 1H), 3.29- 3.26 (m, 2H), 3.02-2.88 (m, 4H), 2.52- 2.52 (m, 4H), 2.27 (s, 3H), 2.21-2.12 (m, 2H), 1.89-1.81 (m, 2H), 1.48-1.39 (m, 1H), 1.35-1.25 (m, 2H), 1.15-1.04 (m, 2H). | 576.29 |
| 79 | 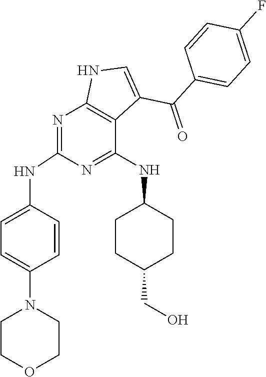 [SMILES: O=C(c1ccc(F)cc1)c1c[nH]c2nc(Nc3ccc(N4CCOCC4)cc3)nc(N[C@H]3CC[C@H](CO)CC3)c12] | ¹H NMR (600 MHz, DMSO-d₆) δ 12.11 (s, 1H), 8.65 (s, 1H), 8.63 (d, J = 7.7 Hz, 1H), 7.88-7.79 (m, 2H), 7.75-7.66 (m, 2H), 7.42 (d, J = 2.8 Hz, 1H), 7.35 (t, J = 8.8 Hz, 2H), 6.84 (d, J = 9.1 Hz, 2H), 4.44 (t, J = 5.3 Hz, 1H), 4.07-3.91 (m, 1H), 3.78-3.71 (m, 4H), 3.30-3.28 (m, 2H), 3.06-2.97 (m, 4H), 2.20-2.10 (m, 2H), 1.89-1.80 (m, 2H), 1.47-1.39 (m, 1H), 1.31-1.24 (m, 2H), 1.13-1.04 (m, 2H). | 545.26 |
| 80 | 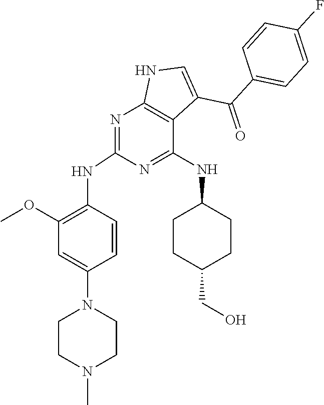 [SMILES: COc1cc(N2CCN(C)CC2)ccc1Nc1nc(N[C@H]2CC[C@H](CO)CC2)c2c(C(=O)c3ccc(F)cc3)c[nH]c2n1] | ¹H NMR (600 MHz, DMSO-d₆) δ 12.15 (s, 1H), 8.66 (d, J = 7.5 Hz, 1H), 8.15 (d, J = 8.8 Hz, 1H), 7.83 (dd, J = 8.6, 5.6 Hz, 2H), 7.43 (s, 1H), 7.34 (t, J = 8.8 Hz, 2H), 7.19 (s, 1H), 6.64 (d, J = 2.3 Hz, 1H), 6.45 (dd, J = 8.8, 2.3 Hz, 1H), 4.44 (t, J = 4.8 Hz, 1H), 4.04-3.89 (m, 1H), 3.86 (s, 3H), 3.29-3.27 (m, 2H), 3.17-3.05 (m, 4H), 2.28 (s, 3H), 2.18-2.08 (m, 2H), 1.89-1.76 (m, 2H), 1.48-1.37 (m, 1H), 1.32-1.22 (m, 2H), 1.14-1.02 (m, 2H). | 588.31 |
| 81 | 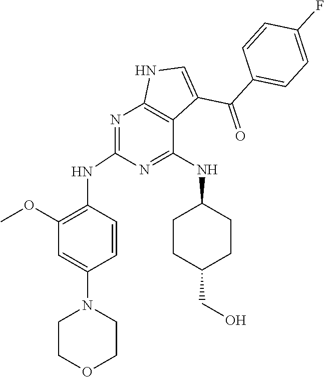 [SMILES: COc1cc(N2CCOCC2)ccc1Nc1nc(N[C@H]2CC[C@H](CO)CC2)c2c(C(=O)c3ccc(F)cc3)c[nH]c2n1] | ¹H NMR (600 MHz, DMSO-d₆) δ 12.16 (s, 1H), 8.67 (d, J = 7.4 Hz, 1H), 8.17 (d, J = 8.9 Hz, 1H), 7.88-7.79 (m, 2H), 7.44 (s, 1H), 7.34 (t, J = 8.8 Hz, 2H), 7.20 (s, 1H), 6.65 (d, J = 2.4 Hz, 1H), 6.46 (dd, J = 8.8, 2.4 Hz, 1H), 4.44 (t, J = 5.3 Hz, 1H), 3.98-3.89 (m, 1H), 3.86 (s, 3H), 3.79-3.72 (m, 4H), 3.29-3.25 (m, 2H), 3.11-3.03 (m, 4H), 2.22-2.06 (m, 2H), 1.91-1.76 (m, 2H), 1.46-1.38 (m, 1H), 1.31-1.22 (m, 2H), 1.13-1.04 (m, 2H). | 575.27 |
| 82 | 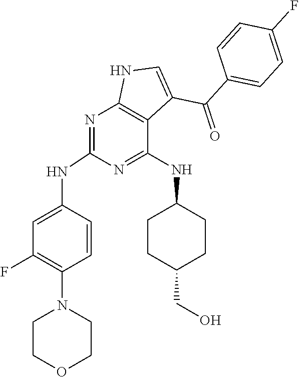 [SMILES: O=C(c1ccc(F)cc1)c1c[nH]c2nc(Nc3ccc(N4CCOCC4)c(F)c3)nc(N[C@H]3CC[C@H](CO)CC3)c12] | ¹H NMR (600 MHz, DMSO-d₆) δ 12.19 (s, 1H), 8.98 (s, 1H), 8.72 (d, J = 7.5 Hz, 1H), 8.02 (dd, J = 16.0, 2.3 Hz, 1H), 7.89- 7.76 (m, 2H), 7.47 (d, J = 3.0 Hz, 1H), 7.40-7.28 (m, 3H), 6.96-6.90 (m, 1H), 4.65-4.33 (m, 1H), 4.01-3.95 (m, 1H), 3.77-3.70 (m, 4H), 3.29-3.27 (m, 2H), 2.96-2.89 (m, 4H), 2.21-2.14 (m, 2H), 1.89-1.82 (m, 2H), 1.48-1.39 (m, 1H), 1.35-1.24 (m, 2H), 1.15-1.05 (m, 2H). | 563.25 |
| 83 | 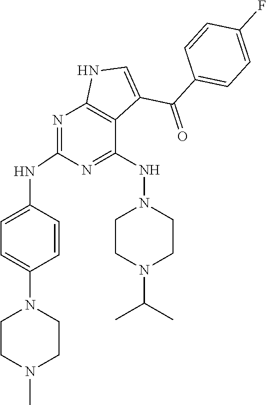 [SMILES: CC(C)N1CCN(Nc2nc(Nc3ccc(N4CCN(C)CC4)cc3)nc3[nH]cc(C(=O)c4ccc(F)cc4)c23)CC1] | ¹H NMR (600 MHz, DMSO-d₆) δ 12.14 (s, 1H), 8.73 (s, 1H), 8.65 (s, 1H), 7.88- 7.78 (m, 2H), 7.70-7.60 (m, 2H), 7.44 (s, 1H), 7.35 (t, J = 8.8 Hz, 2H), 6.84 (d, J = 9.0 Hz, 2H), 4.18-4.01 (m, 1H), 3.31-3.29 (m, 2H), 3.09-3.02 (m, 4H), 3.01-2.64 (m, 3H), 2.48-2.43 (m, 4H), 2.23 (s, 3H), 2.16- 1.99 (m, 2H), 1.68-1.43 (m, 2H), 1.15- 0.93 (m, 6H). | 571.33 |
| 84 | 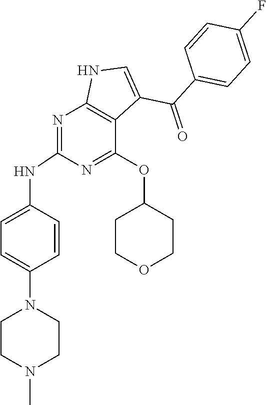 [SMILES: CN1CCN(c2ccc(Nc3nc(OC4CCOCC4)c4c(C(=O)c5ccc(F)cc5)c[nH]c4n3)cc2)CC1] | ¹H NMR (600 MHz, DMSO-d₆) δ 12.15 (s, 1H), 9.02 (s, 1H), 7.89-7.79 (m, 2H), 7.68-7.56 (m, 2H), 7.53 (s, 1H), 7.32 (t, J = 8.8 Hz, 2H), 6.88 (d, J = 9.0 Hz, 2H), 5.35-5.21 (m, 1H), 3.77-3.68 (m, 2H), 3.46-3.38 (m, 2H), 3.12-3.00 (m, 4H), 2.49-2.41 (m, 4H), 2.23 (s, 3H), 1.90-1.80 (m, 2H), 1.39-1.30 (m, 2H). | 531.25 |
| 85 | 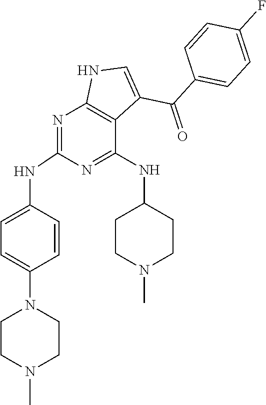 [SMILES: CN1CCC(Nc2nc(Nc3ccc(N4CCN(C)CC4)cc3)nc3[nH]cc(C(=O)c4ccc(F)cc4)c23)CC1] | ¹H NMR (600 MHz, DMSO-d₆) δ 12.15 (s, 1H), 8.72 (d, J = 6.8 Hz, 1H), 8.66 (s, 1H), 7.88-7.79 (m, 2H), 7.69-7.62 (m, 2H), 7.44 (s, 1H), 7.39-7.32 (m, 2H), 6.85 (d, J = 9.1 Hz, 2H), 4.14-4.03 (m, 1H), 3.09-3.00 (m, 4H), 2.93-2.79 (m, 2H), 2.54-2.52 (m, 4H), 2.38-2.26 (m, 5H), 2.24 (s, 3H), 2.13-2.04 (m, 2H), 1.68-1.56 (m, 2H). | 543.30 |
| 86 | 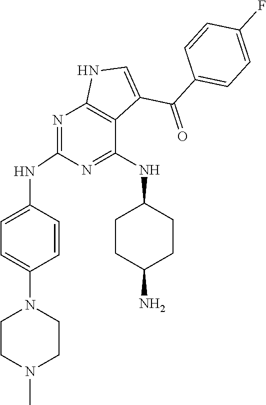 [SMILES: CN1CCN(c2ccc(Nc3nc(N[C@H]4CC[C@@H](N)CC4)c4c(C(=O)c5ccc(F)cc5)c[nH]c4n3)cc2)CC1] | ¹H NMR (600 MHz, DMSO-d₆) δ 12.17 (s, 1H), 8.90 (d, J = 6.5 Hz, 1H), 8.67 (s, 1H), 8.20-7.89 (m, 2H), 7.88-7.82 (m, 2H), 7.71-7.63 (m, 2H), 7.46 (s, 1H), 7.39- 7.33 (m, 2H), 6.85 (d, J = 9.1 Hz, 2H), 4.31-4.19 (m, 1H), 3.22-3.15 (m, 1H), 3.11-3.00 (m, 4H), 2.53-2.52 (m, 4H), 2.27 (s, 3H), 2.06-1.95 (m, 2H), 1.90-1.69 (m, 6H). | 543.30 |
| 87 | 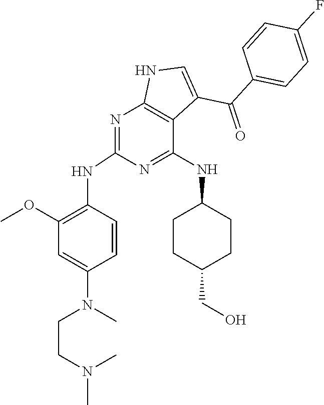 [SMILES: COc1cc(N(C)CCN(C)C)ccc1Nc1nc(N[C@H]2CC[C@H](CO)CC2)c2c(C(=O)c3ccc(F)cc3)c[nH]c2n1] | ND | 590.32 |
| 88 | 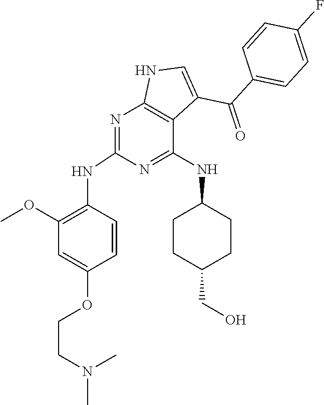 [SMILES: COc1cc(OCCN(C)C)ccc1Nc1nc(N[C@H]2CC[C@H](CO)CC2)c2c(C(=O)c3ccc(F)cc3)c[nH]c2n1] | ND | 577.29 |
| 89 | 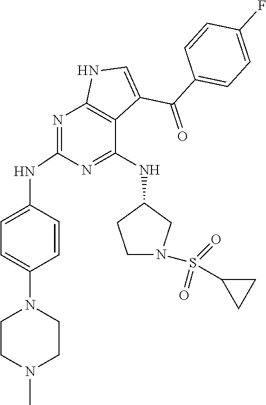 [SMILES: CN1CCN(c2ccc(Nc3nc(N[C@H]4CCN(S(=O)(=O)C5CC5)C4)c4c(C(=O)c5ccc(F)cc5)c[nH]c4n3)cc2)CC1] | ¹H NMR (600 MHz, DMSO-d₆) δ 12.26 (s, 1H), 8.99 (s, 1H), 8.91 (d, J = 6.2 Hz, 1H), 8.58 (s, 1H), 8.01 (dd, J = 9.0, 2.4 Hz, 1H), 7.84 (dd, J = 8.7, 5.6 Hz, 2H), 7.78 (s, 1H), 7.39 (t, J = 8.8 Hz, 2H), 6.83 (d, J = 9.1 Hz, 1H), 4.72-4.64 (m, 1H), 3.79- 3.73 (m, 1H), 3.52-3.39 (m, 5H), 3.33- 3.32 (m, 2H), 2.71-2.67 (m, 1H), 2.59- 2.53 (m, 4H), 2.43-2.39 (m, 1H), 2.33 (s, 3H), 2.09-2.00 (m, 1H), 0.96-0.90 (m, 4H). | 620.25 |
| 90 | 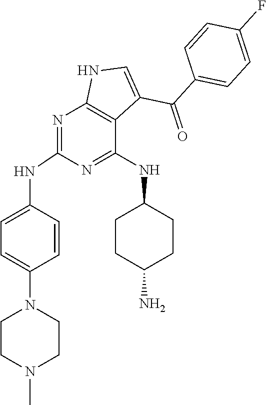 [SMILES: CN1CCN(c2ccc(Nc3nc(N[C@H]4CC[C@H](N)CC4)c4c(C(=O)c5ccc(F)cc5)c[nH]c4n3)cc2)CC1] | ND | 543.30 |
| 91 | 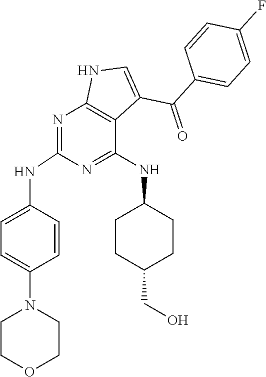 [SMILES: O=C(c1ccc(F)cc1)c1c[nH]c2nc(Nc3ccc(N4CCOCC4)cc3)nc(N[C@H]3CC[C@H](CO)CC3)c12] | ¹H NMR (600 MHz, DMSO-d₆) δ 12.20 (s, 1H), 8.74 (s, 1H), 8.63 (s, 1H), 7.90- 7.78 (m, 1H), 7.69 (d, J = 7.5 Hz, 2H), 7.65-7.49 (m, 3H), 7.00-6.75 (m, 2H), 4.40 (s, 1H), 4.02-3.94 (m, 1H), 3.81-3.71 (m, 4H), 3.29 (d, J = 6.2 Hz, 2H), 3.10- 2.98 (m, 4H), 2.25-2.04 (m, 2H), 1.92- 1.76 (m, 2H), 1.48-1.38 (m, 1H), 1.35- 1.22 (m, 2H), 1.14-1.01 (m, 2H). | 563.25 |
| 92 | 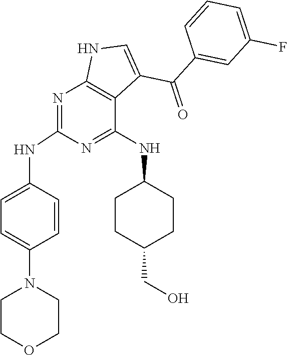 [SMILES: O=C(c1cccc(F)c1)c1c[nH]c2nc(Nc3ccc(N4CCOCC4)cc3)nc(N[C@H]3CC[C@H](CO)CC3)c12] | ¹H NMR (600 MHz, DMSO-d₆) δ 12.17 (s, 1H), 8.73 (s, 1H), 8.62 (s, 1H), 7.69 (d, J = 8.6 Hz, 2H), 7.65-7.55 (m, 2H), 7.40- 7.26 (m, 3H), 6.87 (d, J = 6.7 Hz, 2H), 4.41 (s, 1H), 4.05-3.94 (m, 1H), 3.81-3.71 (m, 4H), 3.29 (d, J = 6.2 Hz, 2H), 3.11- 2.95 (m, 4H), 2.24-2.09 (m, 2H), 1.93- 1.77 (m, 2H), 1.48-1.37 (m, 1H), 1.34- 1.24 (m, 2H), 1.15-1.01 (m, 2H). | 545.26 |
| 93 | 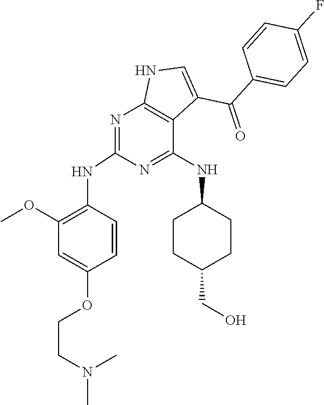 [SMILES: COc1cc(OCCN(C)C)ccc1Nc1nc(N[C@H]2CC[C@H](CO)CC2)c2c(C(=O)c3ccc(F)cc3)c[nH]c2n1] | ND | 576.67 |
| 94 | 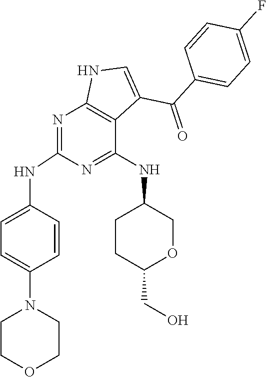 [SMILES: O=C(c1ccc(F)cc1)c1c[nH]c2nc(Nc3ccc(N4CCOCC4)cc3)nc(N[C@@H]3CC[C@@H](CO)OC3)c12] | ¹H NMR (600 MHz, DMSO-d₆) δ 12.19 (s, 1H), 8.76 (s, 1H), 8.57 (d, J = 7.3 Hz, 1H), 7.86-7.80 (m, 2H), 7.69 (d, J = 8.9 Hz, 2H), 7.45 (s, 1H), 7.38-7.29 (m, 2H), 6.86 (d, J = 8.3 Hz, 2H), 4.65 (s, 1H), 4.26- 4.13 (m, 2H), 3.79-3.70 (m, 4H), 3.46- 3.42 (m, 1H), 3.38-3.35 (m, 2H), 3.15- 3.07 (m, 1H), 3.07-2.94 (m, 4H), 2.27- 2.11 (m, 1H), 1.85-1.73 (m, 1H), 1.64- 1.47 (m, 1H), 1.47-1.29 (m, 1H). | 546.60 |
| 95 | 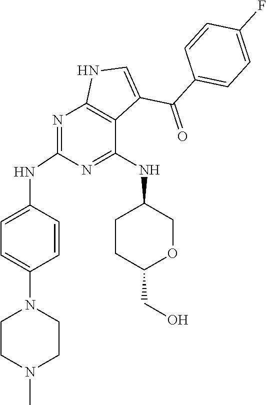 [SMILES: CN1CCN(c2ccc(Nc3nc(N[C@@H]4CC[C@@H](CO)OC4)c4c(C(=O)c5ccc(F)cc5)c[nH]c4n3)cc2)CC1] | ¹H NMR (600 MHz, DMSO-d₆) δ 12.21 (s, 1H), 8.80 (s, 1H), 8.56 (d, J = 7.5 Hz, 1H), 7.89-7.80 (m, 2H), 7.79-7.68 (m, 2H), 7.46 (d, J = 2.7 Hz, 1H), 7.35 (t, J = 8.8 Hz, 2H), 6.89 (d, J = 9.1 Hz, 2H), 4.65 (s, 1H), 4.25-4.11 (m, 2H), 3.48-3.41 (m, 1H), 3.29-3.15 (m, 4H), 3.15-3.06 (m, 4H), 2.69 (s, 3H), 2.24-2.13 (m, 1H), 1.86- 1.75 (m, 1H), 1.61-1.47 (m, 1H), 1.47- 1.32 (m, 1H). | 559.65 |
| 96 | 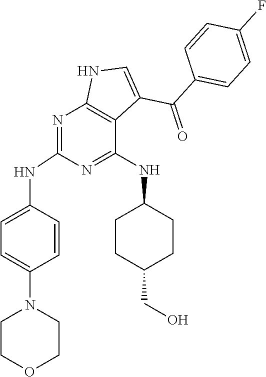 [SMILES: O=C(c1ccc(F)cc1)c1c[nH]c2nc(Nc3ccc(N4CCOCC4)cc3)nc(N[C@H]3CC[C@H](CO)CC3)c12] | ¹H NMR (600 MHz, DMSO-d₆) δ 12.18 (s, 1H), 8.75 (s, 1H), 8.63 (s, 1H), 7.69 (d, J = 8.5 Hz, 2H), 7.63-7.55 (m, 2H), 7.40- 7.25 (m, 3H), 6.88 (d, J = 5.8 Hz, 2H), 4.42 (s, 1H), 4.05-3.94 (m, 1H), 3.78-3.72 (m, 4H), 3.29 (d, J = 6.2 Hz, 2H), 3.10- 2.98 (m, 4H), 2.24-2.09 (m, 2H), 1.92- 1.77 (m, 2H), 1.49-1.38 (m, 1H), 1.36- 1.25 (m, 2H), 1.14-1.04 (m, 2H). | 562.62 |
| 97 | 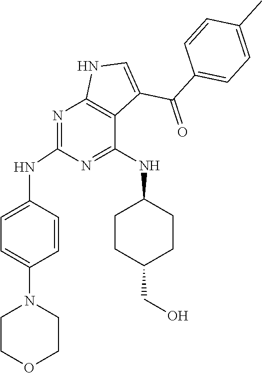 [SMILES: Cc1ccc(C(=O)c2c[nH]c3nc(Nc4ccc(N5CCOCC5)cc4)nc(N[C@H]4CC[C@H](CO)CC4)c23)cc1] | ¹H NMR (600 MHz, DMSO-d₆) δ 12.15 (s, 1H), 9.39-8.51 (m, 2H), 7.78-7.60 (m, 4H), 7.43 (s, 1H), 7.34 (d, J = 7.9 Hz, 2H), 6.92 (d, J = 6.1 Hz, 2H), 4.44 (s, 1H), 4.03- 3.93 (m, 1H), 3.80-3.73 (m, 4H), 3.29 (d, J = 6.2 Hz, 2H), 3.12-3.02 (m, 4H), 2.41 (s, 3H), 2.20-2.09 (m, 2H), 1.89-1.80 (m, 2H), 1.51-1.39 (m, 1H), 1.35-1.25 (m, 2H), 1.13-1.03 (m, 2H). | 541.32 |
| 98 | 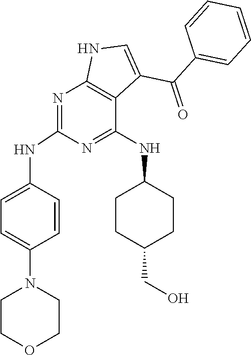 [SMILES: O=C(c1ccccc1)c1c[nH]c2nc(Nc3ccc(N4CCOCC4)cc3)nc(N[C@H]3CC[C@H](CO)CC3)c12] | ¹H NMR (600 MHz, DMSO-d₆) δ 12.20 (s, 1H), 9.28-8.55 (m, 2H), 7.79-7.74 (m, 2H), 7.69 (d, J = 7.9 Hz, 2H), 7.65-7.60 (m, 1H), 7.54 (t, J = 7.6 Hz, 2H), 7.43 (s, 1H), 6.93 (d, J = 6.0 Hz, 2H), 4.24 (s, 1H), 4.01-3.95 (m, 1H), 3.79-3.74 (m, 4H), 3.29 (d, J = 6.2 Hz, 2H), 3.16-3.02 (m, 4H), 2.24-2.09 (m, 2H), 1.93-1.77 (m, 2H), 1.49-1.39 (m, 1H), 1.36-1.26 (m, 2H), 1.14-1.04 (m, 2H). | 527.33 |
| 99 | 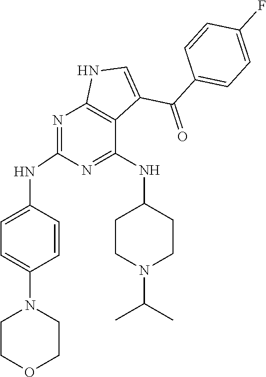 [SMILES: CC(C)N1CCC(Nc2nc(Nc3ccc(N4CCOCC4)cc3)nc3[nH]cc(C(=O)c4ccc(F)cc4)c23)CC1] | ¹H NMR (600 MHz, DMSO-d₆) δ 12.28 (s, 1H), 9.19 (s, 1H), 8.81 (d, J = 8.6 Hz, 1H), 7.85-7.82 (m, 2H), 7.66 (d, J = 8.8 Hz, 2H), 7.51 (s, 1H), 7.38-7.35 (m, 2H), 6.94 (d, J = 8.9 Hz, 2H), 4.25-4.16 (m, 1H), 3.77-3.75 (m, 4H), 3.55-3.52 (m, 2H), 3.27-3.18 (m, 1H), 3.18-3.10 (m, 2H), 3.08-3.05 (m, 4H), 2.47-2.40 (m, 2H), 1.87-1.72 (m, 2H), 1.30 (d, J = 6.6 Hz, 6H). | 558.33 |
| 100 | 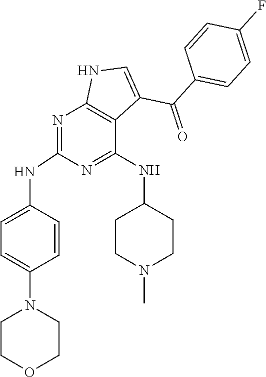 [SMILES: CN1CCC(Nc2nc(Nc3ccc(N4CCOCC4)cc3)nc3[nH]cc(C(=O)c4ccc(F)cc4)c23)CC1] | ND | 530.29 |
| 101 | 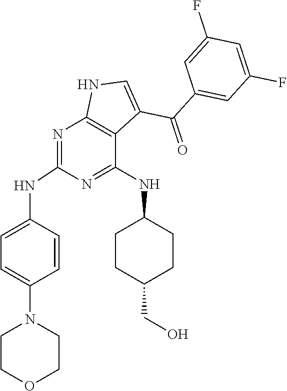 [SMILES: O=C(c1cc(F)cc(F)c1)c1c[nH]c2nc(Nc3ccc(N4CCOCC4)cc3)nc(N[C@H]3CC[C@H](CO)CC3)c12] | ¹H NMR (600 MHz, DMSO-d₆) δ 12.20 (s, 1H), 8.68 (s, 1H), 8.54 (d, J = 7.5 Hz, 1H), 7.68 (d, J = 8.8 Hz, 2H), 7.56 (s, 1H), 7.50 (t, J = 9.2 Hz, 1H), 7.44 (d, J = 5.6 Hz, 2H), 6.85 (d, J = 8.9 Hz, 2H), 4.44 (t, J = 5.2 Hz, 1H), 4.07-3.94 (m, 1H), 3.78- 3.69 (m, 4H), 3.30-3.27 (m, 2H), 3.08- 2.96 (m, 4H), 2.21-2.09 (m, 2H), 1.89- 1.78 (m, 2H), 1.49-1.37 (m, 1H), 1.33- 1.21 (m, 2H), 1.14-1.03 (m, 2H). | 563.30 |
| 102 | 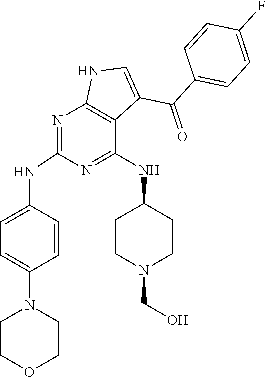 | ¹H NMR (600 MHz, DMSO-d₆) δ 12.17 (s, 1H), 9.06 (s, 1H), 8.76 (s, 1H), 7.92- 7.78 (m, 2H), 7.69 (d, J = 7.9 Hz, 2H), 7.45 (s, 1H), 7.35 (t, J = 8.7 Hz, 2H), 6.90 (d, J = 6.2 Hz, 2H), 4.40 (s, 1H), 3.81-3.71 (m, 4H), 3.30 (d, J = 6.6 Hz, 2H), 3.12- 2.97 (m, 4H), 1.89-1.79 (m, 2H), 1.73- 1.62 (m, 4H), 1.59-1.49 (m, 1H), 1.44- 1.33 (m, 2H). | 545.32 |
| 103 | 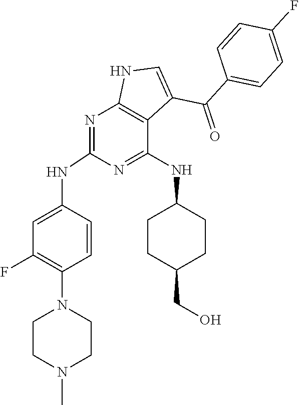 [SMILES: CN1CCN(c2ccc(Nc3nc(N[C@H]4CC[C@@H](CO)CC4)c4c(C(=O)c5ccc(F)cc5)c[nH]c4n3)cc2F)CC1] | ¹H NMR (600 MHz, DMSO-d₆) δ 12.26 (s, 1H), 9.11 (s, 1H), 9.08 (d, J = 7.4 Hz, 1H), 8.05-7.96 (m, 1H), 7.90-7.80 (m, 2H), 7.51 (s, 1H), 7.46-7.32 (m, 3H), 7.01 (t, J = 9.4 Hz, 1H), 4.44-4.36 (m, 1H), 3.54-3.49 (m, 1H), 3.44-3.37 (m, 2H), 3.31 (d, J = 6.7 Hz, 2H), 3.26-3.19 (m, 2H), 3.03-2.95 (m, 2H), 2.88 (s, 3H), 1.91- 1.81 (m, 2H), 1.74-1.64 (m, 4H), 1.57- 1.49 (m, 1H), 1.46-1.34 (m, 2H). | 576.33 |
| 104 | 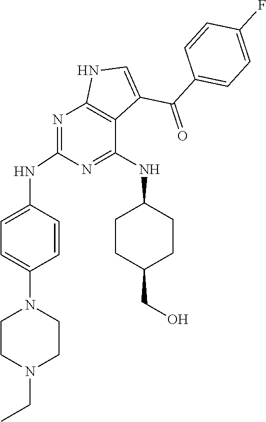 [SMILES: CCN1CCN(c2ccc(Nc3nc(N[C@H]4CC[C@@H](CO)CC4)c4c(C(=O)c5ccc(F)cc5)c[nH]c4n3)cc2)CC1] | ¹H NMR (600 MHz, DMSO-d₆) δ 12.11 (s, 1H), 8.96 (d, J = 7.5 Hz, 1H), 8.65 (s, 1H), 7.90-7.77 (m, 2H), 7.68 (d, J = 8.5 Hz, 2H), 7.43 (s, 1H), 7.35 (t, J = 8.7 Hz, 2H), 6.86 (d, J = 8.4 Hz, 2H), 4.45 (t, J = 5.1 Hz, 1H), 4.43-4.36 (m, 1H), 3.31-3.23 (m, 4H), 3.19-2.94 (m, 4H), 2.84-2.54 (m, 4H), 1.90-1.80 (m, 2H), 1.72-1.61 (m, 4H), 1.58-1.48 (m, 1H), 1.44-1.34 (m, 2H), 1.15-1.01 (m, 3H). | 572.35 |
| 105 | 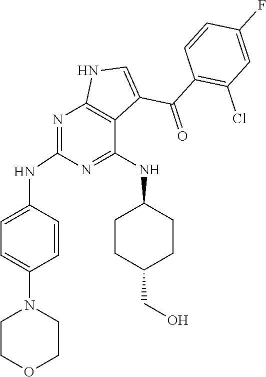 [SMILES: O=C(c1ccc(F)cc1Cl)c1c[nH]c2nc(Nc3ccc(N4CCOCC4)cc3)nc(N[C@H]3CC[C@H](CO)CC3)c12] | ¹H NMR (600 MHz, DMSO-d₆) δ 12.35 (s, 1H), 8.84 (s, 1H), 7.68-7.62 (m, 2H), 7.28-7.20 (m, 1H), 6.93-6.77 (m, 4H), 5.54-5.38 (m, 1H), 4.05-3.89 (m, 1H), 3.78-3.70 (m, 4H), 3.30-3.26 (m, 2H), 3.05-2.98 (m, 4H), 2.17-2.07 (m, 2H), 1.88-1.78 (m, 2H), 1.47-1.38 (m, 1H), 1.33-1.20 (m, 2H), 1.14-1.02 (m, 2H). | 579.22815 |
| 106 | 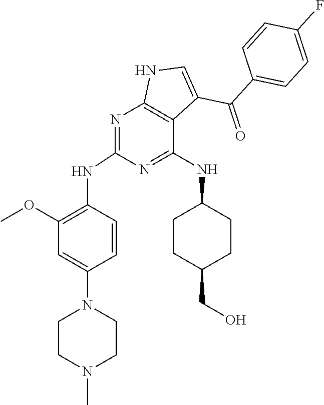 [SMILES: COc1cc(N2CCN(C)CC2)ccc1Nc1nc(N[C@H]2CC[C@@H](CO)CC2)c2c(C(=O)c3ccc(F)cc3)c[nH]c2n1] | ¹H NMR (600 MHz, DMSO-d₆) δ 12.17 (s, 1H), 8.99 (d, J = 7.2 Hz, 1H), 8.19 (d, J = 8.5 Hz, 1H), 7.93-7.77 (m, 2H), 7.45 (s, 1H), 7.35 (t, J = 8.6 Hz, 2H), 7.19 (s, 1H), 6.64 (s, 1H), 6.48 (d, J = 8.5 Hz, 1H), 4.56-4.40 (m, 1H), 4.41-4.30 (m, 1H), 3.86 (s, 3H), 3.30-3.28 (m, 2H), 3.20-3.02 (m, 4H), 2.56-2.52 (m, 4H), 2.28 (s, 3H), 1.89-1.80 (m, 2H), 1.72-1.60 (m, 4H), 1.58-1.47 (m, 1H), 1.43-1.32 (m, 2H). | 588.30927 |
| 107 | 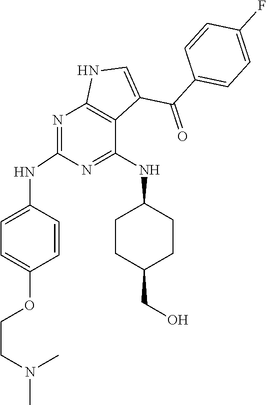 [SMILES: CN(C)CCOc1ccc(Nc2nc(N[C@H]3CC[C@@H](CO)CC3)c3c(C(=O)c4ccc(F)cc4)c[nH]c3n2)cc1] | ¹H NMR (600 MHz, DMSO-d₆) δ 12.13 (s, 1H), 8.98 (d, J = 7.6 Hz, 1H), 8.78 (s, 1H), 7.84 (dd, J = 8.3, 5.7 Hz, 2H), 7.76 (d, J = 9.0 Hz, 2H), 7.45 (s, 1H), 7.35 (t, J = 8.7 Hz, 2H), 6.91 (d, J = 8.9 Hz, 2H), 4.46 (t, J = 5.2 Hz, 1H), 4.42-4.35 (m, 1H), 4.29-4.18 (m, 2H), 3.40-3.37 (m, 2H), 3.30-3.28 (m, 2H), 2.79 (s, 6H), 1.90-1.80 (m, 2H), 1.72-1.60 (m, 4H), 1.58-1.50 (m, 1H), 1.45-1.33 (m, 2H). | 547.28 |
| 108 | 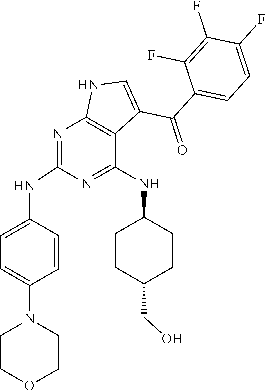 [SMILES: O=C(c1ccc(F)c(F)c1F)c1c[nH]c2nc(Nc3ccc(N4CCOCC4)cc3)nc(N[C@H]3CC[C@H](CO)CC3)c12] | ¹H NMR (600 MHz, DMSO-d₆) δ 12.25 (s, 1H), 8.70 (s, 1H), 8.43 (d, J = 7.5 Hz, 1H), 7.68 (dd, J = 9.1, 3.4 Hz, 2H), 7.54 (s, 1H), 7.51-7.40 (m, 2H), 6.85 (d, J = 9.1 Hz, 2H), 4.45 (t, J = 5.3 Hz, 1H), 4.02- 3.96 (m, 1H), 3.76-3.72 (m, 4H), 3.29 (t, J = 5.7 Hz, 2H), 3.03-2.99 (m, 4H), 2.20- 2.11 (m, 2H), 1.88-1.81 (m, 2H), 1.47- 1.39 (m, 1H), 1.32-1.23 (m, 2H), 1.13- 1.05 (m, 2H). | 581.30 |
| 109 | 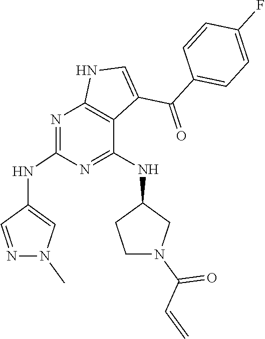 [SMILES: C=CC(=O)N1CC[C@@H](Nc2nc(Nc3cnn(C)c3)nc3[nH]cc(C(=O)c4ccc(F)cc4)c23)C1] | ¹H NMR (600 MHz, DMSO-d₆) δ 12.18 (s, 1H), 8.93 (d, J = 9.8 Hz, 1H), 8.86 (s, 1H), 7.93-7.71 (m, 3H), 7.52 (s, 1H), 7.48 (d, J = 2.8 Hz, 1H), 7.35 (t, J = 8.8 Hz, 2H), 6.62 (ddd, J = 42.2, 16.7, 10.3 Hz, 1H), 6.15 (ddd, J = 16.8, 5.8, 2.4 Hz, 1H), 5.68 (ddd, J = 17.9, 10.3, 2.4 Hz, 1H), 4.82-4.57 (m, 1H), 4.08-3.76 (m, 5H), 3.68-3.45 (m, 2H), 2.44-2.26 (m, 1H), 2.17-1.93 (m, 1H). | 475.22 |
| 110 | 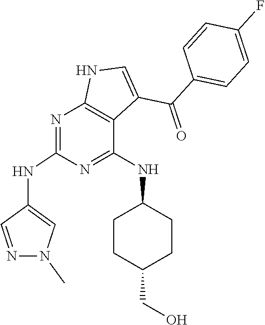 [SMILES: Cn1cc(Nc2nc(N[C@H]3CC[C@H](CO)CC3)c3c(C(=O)c4ccc(F)cc4)c[nH]c3n2)cn1] | ¹H NMR (600 MHz, DMSO-d₆) δ 12.05 (s, 1H), 8.72 (s, 1H), 8.67 (d, J = 4.6 Hz, 1H), 7.93-7.76 (m, 3H), 7.51 (s, 1H), 7.41 (d, J = 2.7 Hz, 1H), 7.37-7.32 (m, 2H), 4.72-4.24 (m, 1H), 4.01-3.92 (m, 1H), 3.79 (s, 3H), 3.30-3.26 (m, 2H), 2.25-2.08 (m, 2H), 1.91-1.79 (m, 2H), 1.50-1.38 (m, 1H), 1.33-1.22 (m, 2H), 1.17-1.04 (m, 2H). | 464.22 |
| 111 | 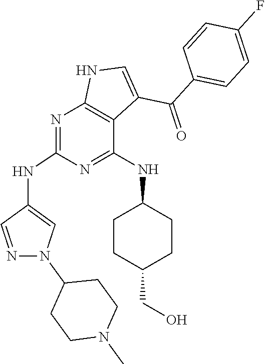 [SMILES: CN1CCC(n2cc(Nc3nc(N[C@H]4CC[C@H](CO)CC4)c4c(C(=O)c5ccc(F)cc5)c[nH]c4n3)cn2)CC1] | ND | 547.29 |
| 112 | 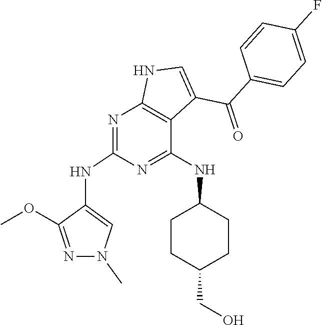 [SMILES: COc1nn(C)cc1Nc1nc(N[C@H]2CC[C@H](CO)CC2)c2c(C(=O)c3ccc(F)cc3)c[nH]c2n1] | ¹H NMR (600 MHz, DMSO-d₆) δ 12.10 (s, 1H), 8.59 (d, J = 6.3 Hz, 1H), 7.85-7.78 (m, 2H), 7.66 (s, 1H), 7.43 (s, 1H), 7.37 (s, 1H), 7.33 (t, J = 8.8 Hz, 2H), 4.43 (s, 1H), 3.99-3.84 (m, 1H), 3.79 (s, 3H), 3.66 (s, 3H), 3.29-3.24 (m, 2H), 2.20-2.05 (m, 2H), 1.87-1.78 (m, 2H), 1.48-1.35 (m, 1H), 1.29-1.21 (m, 2H), 1.11-1.00 (m, 2H). | 494.26 |
| 113 | 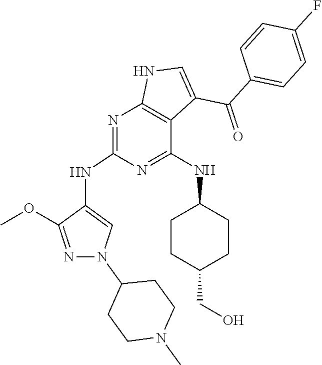 [SMILES: COc1nn(C2CCN(C)CC2)cc1Nc1nc(N[C@H]2CC[C@H](CO)CC2)c2c(C(=O)c3ccc(F)cc3)c[nH]c2n1] | ND | 577.36 |
| 114 | 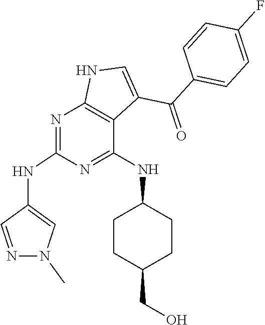 [SMILES: Cn1cc(Nc2nc(N[C@H]3CC[C@@H](CO)CC3)c3c(C(=O)c4ccc(F)cc4)c[nH]c3n2)cn1] | ¹H NMR (600 MHz, DMSO-d₆) δ 12.19 (s, 1H), 9.21 (s, 1H), 8.93 (s, 1H), 7.95- 7.77 (m, 3H), 7.53 (s, 1H), 7.47 (s, 1H), 7.36 (t, J = 8.6 Hz, 2H), 4.43-4.33 (m, 1H), 3.80 (s, 3H), 3.30 (d, J = 6.4 Hz, 2H), 1.93- 1.81 (m, 2H), 1.74-1.61 (m, 4H), 1.59- 1.49 (m, 1H), 1.44-1.30 (m, 2H). | 464.22 |
| 115 | 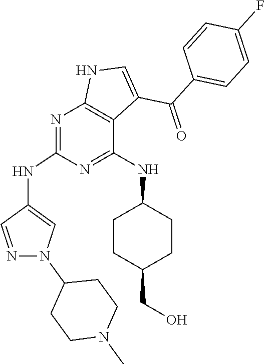 [SMILES: CN1CCC(n2cc(Nc3nc(N[C@H]4CC[C@@H](CO)CC4)c4c(C(=O)c5ccc(F)cc5)c[nH]c4n3)cn2)CC1] | ND | 547.28259 |
| 116 | 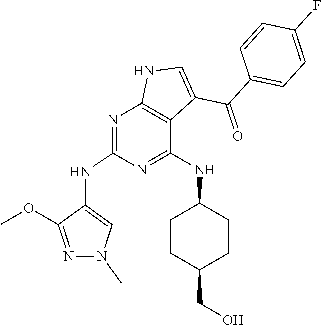 [SMILES: COc1nn(C)cc1Nc1nc(N[C@H]2CC[C@@H](CO)CC2)c2c(C(=O)c3ccc(F)cc3)c[nH]c2n1] | ¹H NMR (600 MHz, DMSO-d₆) δ 12.11 (s, 1H), 8.90 (d, J = 7.6 Hz, 1H), 7.82 (dd, J = 8.6, 5.6 Hz, 2H), 7.64 (s, 1H), 7.43- 7.29 (m, 4H), 4.43 (t, J = 5.3 Hz, 1H), 4.39-4.27 (m, 1H), 3.78 (s, 3H), 3.66 (s, 3H), 3.30-3.24 (m, 2H), 1.85-1.77 (m, 2H), 1.70-1.57 (m, 4H), 1.56-1.46 (m, 1H), 1.42-1.31 (m, 2H). | 494.23 |

### Example 117 In-Vitro Enzyme Experiment 1

The compound of the present invention had a good inhibitory activity on an EGFR kinase, and the IC50 values were all less than 10 μM. The specific experiment was as follows:
1. Preparation of 1× concentration kinase buffer
1 volume of 5 times concentration of an enzyme buffer diluted with 4 volumes of distilled water; 5 mM magnesium chloride; 1 mM DTT; and 1 mM MnCl₂.
2. Test method of activity of compound
a) a compound diluent was transferred to each well of an assay plate (784075, Greiner) using Echo 550;
b) the assay plate was sealed and centrifuged at 1,000 g for 1 min;
c) 2×EGFR (D770-N771 ins NPG T790M) was prepared in a 1× kinase buffer;
d) 5 μl of the 2×EGFR (D770-N771 ins NPG T790M) was added to a 384-well assay plate (784075, Greiner);
e) the assay plate was centrifuged at 1,000 g for 30 s and incubated at room temperature for 10 min;
f) 2× TK-substrate-biotin (2 μM) and ATP were added to the 1× kinase buffer and mixed;
g) the reaction was started after adding 5 μl of the TK-substrate-biotin (2 μM) and ATP mixture;
h) the assay plate was centrifuged at 1,000 g for 30 s; and the assay plate was sealed and incubated at room temperature for 40 min;
i) 4× Sa-XL 665 was prepared in an HTRF detection buffer;
j) 5 μl of the Sa-XL 665 and 5 μl of a TK-antibody-cryptate were added to each well of the assay plate;
k) the assay plate was centrifuged at 1,000 g for 30 s and incubated at room temperature for 1 h; and
l) fluorescence signals at 615 nm (cryptate) and 665 nm (XL665) were read on an Envision 2104 reader.
3. Data analysis
3.1 Calculation of ratio (665/615 nm) of each well
3.2 Calculation of inhibition rate

$$Inhibition rate = 100 - ( signal mpd - Signal Ave\_PC ) / ( Signal Ave\_VC - Signal Ave\_PC ) \times 100.$$

3.3 Calculation of IC₅₀ value of compound and drawing dose-effect curve of compound
IC₅₀ values were calculated using GraphPad 6.0 by applying a non-linear regression curve (dose-effect relationship-variable slope) of the logarithm of the compound concentration versus the inhibition rate.

Y=lowest value+(highest value−lowest value)/(1+10\hat{ }((Log IC₅₀−X)×slope))
X: logarithm of inhibitor concentration; and Y: inhibition rate.
3.4 Double test report
3.4.1 After one tester completed a report, another tester again checked the report to ensure that data analysis was correct.
3.4.2 The data was exported from the software and manually analyzed.
3.4.2.1 The rate was converted into the inhibition rate. All the IC₅₀ values were first calculated with the inhibition rate using Prism GraphPad 6.0.
3.4.2.2 Then the IC₅₀ value was recalculated using the ratio to check the accuracy of the analysis.
3.4.3 The number of all the compounds was ensured to be correct.

The activity of the compound of the present invention on a D770-N771 ins NPG kinase was determined by using the method.

The results of activity of some compounds were shown in Table 1.

TABLE 1
Some compounds of the present invention on activity of
D770-N771 ins NPG/T790M kinase and D770-N771 ins
NPG kinase and BaF3-EGFR D770-N771 ins NPG cells

| | D770-N771 | | KC-1362 |
|---|---|---|---|
| | ins | D770-N771 | BaF3-EGFR |
| | NPG/T790M | ins NPG | D770-N771 |
| Example | kinase | kinase | ins NPG cells |
| No. | IC₅₀ (nM) | IC₅₀ (nM) | IC₅₀ (μM) |
| 60 | 8.80 | 2.82 | 0.070 |
| 114 | 18.21 | 4.12 | 0.16 |
| 115 | 8.16 | 2.46 | 0.56 |
| 71 | 9.27 | 4.54 | 0.070 |
| 104 | 11.39 | 2.72 | 0.16 |
| 18 | ND | 28.20 | 0.15 |
| 64 | 93.64 | 24.25 | 0.20 |
| 109 | 7.27 | 3.00 | 0.12 |
| 26 | 6.31 | 3.52 | 0.15 |
| 70 | ND | 6.55 | 0.054 |
| 42 | ND | 4.44 | 0.035 |

ND indicated not detected

The compound of the present invention had significant inhibitory activity and anti-proliferative activity on EGFR D770-N771 ins NPG and EGFR D770-N771 ins NPG/T790M kinases and BaF3-EGFR D770-N771 ins NPG cells carrying an insertion mutation. It was suggested that the compound of the present invention can inhibit EGFR D770-N771 ins NPG mutation and EGFR D770-N771 ins NPG/T790M mutation.

### Example 118 In-Vitro Enzyme Experiment 2

The preparation of the kinase buffer, the test method of the activity of the compound, and the data analysis referred to the relevant definitions in example 117, wherein step c) of the test method of the activity of the compound was replaced by preparing 2×EGFR Del19 T790M C797S in the 1× kinase buffer.

The results of activity of some compounds were shown in Table 2.

TABLE 2
Results of enzyme inhibition IC₅₀ (nM)

| Target | | Target | |
|---|---|---|---|
| compound | EGFR- | compound | EGFR- |
| corresponding | Del19/ | corresponding | Del19/ |
| to example | T790M/ | to example | T790M/ |
| No. | C797S | No. | C797S |
| 41 | C | 110 | B |
| 43 | C | 84 | C |
| 49 | C | 85 | C |
| 50 | C | 86 | A |
| 55 | B | Osimertinib | C |
| 56 | B | 94 | B |
| 57 | C | 95 | B |
| 58 | C | 96 | B |
| 59 | C | 112 | C |
| 60 | A | 18 | B |
| 63 | C | 98 | C |
| 64 | C | 102 | B |
| 67 | C | 103 | B |
| 68 | C | 104 | A |
| 70 | B | 114 | A |
| 71 | B | 106 | B |
| 10 | B | 115 | A |
| 11 | C | 107 | B |
| 73 | C | 116 | B |
| 78 | B | 19 | B |
| 80 | C | 20 | B |
| 82 | C | 108 | C |

A: <10 nM;
B: 10-100 nM; and
C: 100-600 nM.
The compound of the present invention had a very good inhibition effect on an EGFR-Del19/T790M/C797S kinase.

### Example 119 Cell Proliferation Inhibition Experiment

### 1. Experimental Grouping

Experimental group (As): cell+medium+drug+cck-8; control group (A₀): cell +medium+cck-8, and no drug; and blank group (Ac): medium+cck-8, and no cell and no drug.

### 2. Experimental Cell Lines

### Human Lung Cancer Cells:

HCC827^{EGFR-Del19};
PC-9^{EGFR-Del19}.
H1975^{EGFR-L858R/T790M};
A549^{EGFR-WT};
KC-0122:BaF3^{EGFR-L858R/T790M/C797S};
KC-0116:BaF3^{EGFR-Del19/T790M/C797S};
KC-1474:PC-9^{EGFR-Del19/T790M/C797S}; and
KC-0178:NCI-1975^{EGFR-L858R/T790M/C797S}.

### 3. Cell Culture

### 3.1. Cell Recovery

Cryopreservation tubes of cells were taken out from liquid nitrogen, immediately put into warm water at 37° C., and quickly shaken until cryopreserved liquid was completely dissolved; the cell cryopreserved liquid was transferred into a centrifuge tube, about 3 ml of a culture solution was added, an obtained mixture was gently pipetted, uniformly mixed, and centrifuged at 1,000 r/min for 5 min; and a supernatant was discarded, and an appropriate amount of the culture solution was added, and the cells were transferred to a culture flask for culture.

### 3.2 Cell Passage

When the cell covering rate in the culture flask reached 80-90%, the original culture solution was sucked and a proper amount of pancreatin was added; after the cells became round, the pancreatin was discarded, an appropriate amount of the culture solution was added, and an obtained mixture was pipetted and uniformly mixed; and an appropriate amount of a cell suspension was discarded, an equal amount of the culture solution was added, and the cells were placed into an incubator for continuous culture.

### 4. Effect of Drug on Cell Line

The cells in the logarithmic growth phase were taken, the density of the cells was adjusted to be 2-3×10₄ cells/ml by using the culture solution, the cells were inoculated into a 96-well plate with 100 μl per well, and 200 μl of normal saline per well was added on the outermost periphery. After the cells were cultured in the incubator for 24 h, different concentrations of compounds were added, 6 replicate wells were set per concentration, and 100 μl of the compounds was administered per well. The cells were cultured for 72 h and then a CCK-8 solution was added at 15 al/well. After the cells were continuously cultured for 2 h, the absorbance value was detected at the wavelength of 450 nm using a microplate reader. Inhibition rate %=[(A0−AS)/(A0−AC)]×100%. The IC₅₀ values of the compounds on the cells were calculated using an SPSS software.

### 5. Test Results

TABLE 3
Results of cell proliferation inhibition experiment IC₅₀ (μM)

| Target | | | | | | |
|---|---|---|---|---|---|---|
| compound | | | | | | |
| corresponding | | | | | | |
| to example | KC- | KC- | | | | |
| No. | 1474 | 0178 | HCC827 | PC-9 | H1975 | A549 |
| 40 | * | ND | ** | ** | ND | ** |
| 42 | * | *** | ** | * | ** | *** |
| 43 | ND | ND | ** | ** | ** | ND |
| 46 | ND | ND | ** | * | * | ND |
| 109 | ND | ND | ** | * | ** | ** |
| 55 | * | ** | ND | * | ** | ** |
| 56 | * | *** | ND | * | ** | ** |
| 60 | * | *** | ND | * | ** | ** |
| 78 | * | *** | ND | * | ** | ND |
| 79 | ND | * | * | ** | ** | ** |
| 110 | ** | * | * | * | ND | ** |
| 86 | * | *** | ND | * | ** | ND |
| 92 | ND | ND | * | ** | * | ND |
| 95 | ** | * | ND | ND | ND | ND |
| 97 | ** | ** | ND | ND | ND | ND |
| 102 | ** | ** | ND | * | ** | ** |
| 103 | * | *** | ND | * | ** | ** |
| 104 | * | ** | ND | * | * | ND |
| 114 | * | ** | ND | * | * | ** |
| 106 | ** | *** | ND | ND | ND | ND |
| 115 | ** | *** | ND | ** | ** | *** |
| 107 | ** | *** | ND | * | ** | ND |
| 116 | ** | *** | ND | * | ** | *** |
| 19 | ** | *** | ND | * | ** | ** |
| 20 | ** | *** | ND | * | ** | ** |
| Avitinib | ** | *** | ND | ND | ND | ND |
| Osimertinib | *** | *** | ND | ND | ND | ND |

*: <0.1 μM;
**: 0.1-1.0 μM;
***: 1.0-2.0 μM; and
ND represented not detected.

The compound of the present invention has a very good inhibitory effect on the cell proliferation of HCC827^{EGF-Del19} and PC-9^{EGFR-Dell9} mutant cell lines, and an H1975^{EGEF-L858R/T790M} double mutant cell line.

The compound of the present invention has a very good inhibitory effect on the cell proliferation of a KC-1474:PC-9^{EGR-Del19/T790M/C797S} three-mutation cell line and a KC-0178:NC-1975^{EGFR-L858R/T790M/C797S} three-mutation cell line.

TABLE 4
Results of cell proliferation
inhibition experiment IC₅₀ (μM)

| Target | | | Target | | |
|---|---|---|---|---|---|
| compound | | | compound | | |
| corresponding | | | corresponding | | |
| to example | KC- | KC- | to example | KC- | KC- |
| No. | 0122 | 0116 | No. | 0122 | 0116 |
| 22 | *** | ** | 72 | ** | ** |
| 23 | ** | ** | 10 | ** | ** |
| 24 | ** | ** | 74 | ** | ** |
| 25 | ** | ** | 75 | ** | ** |
| 27 | ** | ** | 12 | ** | ** |
| 28 | *** | ND | 13 | * | * |
| 30 | ** | * | 76 | ** | ** |
| 31 | ** | ND | 77 | ** | ** |
| 32 | ND | ** | 14 | ** | ** |
| 33 | ND | ** | 78 | * | ** |
| 34 | ND | ** | 79 | * | * |
| 35 | ND | ** | 80 | ND | * |
| 36 | ND | ** | 81 | ND | * |
| 37 | ND | ** | 82 | ND | * |
| 38 | ND | ** | 110 | ** | * |
| 39 | ND | ** | 86 | ** | * |
| 40 | * | * | 15 | ND | ** |
| 42 | * | * | 16 | ND | ** |
| 43 | ** | ** | 89 | ND | * |
| 44 | ** | ** | 90 | ND | ** |
| 45 | ** | ** | 111 | ND | ** |
| 46 | ** | ** | 91 | * | * |
| 109 | *** | *** | 92 | * | * |
| 47 | ND | *** | 95 | ** | ** |
| 48 | ** | ** | 96 | ND | * |
| 51 | ** | ** | 113 | ND | ** |
| 52 | ** | ** | 97 | * | * |
| 53 | ** | ** | 98 | * | * |
| 54 | ND | ** | 99 | ND | * |
| 55 | * | * | 100 | ND | * |
| 56 | * | * | 101 | * | * |
| 59 | * | * | 102 | * | ** |
| 60 | * | * | 103 | ** | ** |
| 2 | *** | ** | 104 | * | * |
| 62 | ** | ** | 14 | * | ** |
| 64 | * | * | 105 | ND | * |
| 65 | ** | ** | 106 | * | ** |
| 66 | ** | ** | 115 | ** | ** |
| 69 | ** | ** | 107 | ** | ** |
| 5 | ** | ** | 116 | ** | ** |
| 70 | * | * | 19 | ** | ** |
| 71 | ** | * | 20 | ** | ** |
| 6 | ** | ** | Brigatinib | ** | ** |
| 7 | ** | ** | Avitinib | *** | ** |
| 8 | ** | ** | Osimertinib | *** | *** |
| 9 | ** | ** | | | |

*: <0.1 μM;
**: 0.1-1.0 μM;
***: 1.0-2.0 μM; and ND represented not detected.

The compound of the present invention has a very good inhibitory effect on the cell proliferation of a KC-0122:Ba/F3^{EGFR-L58R/T790M/C797S} three-mutation cell line and a KC-0116:Ba/F3^{EGFR-Del19/T79OM/C797S} three-mutation cell line.

TABLE 5
Results of cell proliferation inhibition experiment IC₅₀ (μM)

| Target | | | | Target | | | |
|---|---|---|---|---|---|---|---|
| compound | | | | compound | | | |
| corresponding | | | | corresponding | | | |
| to example | | KC- | KC- | to example | | KC- | KC- |
| No. | A549 | 0122 | 0116 | No. | A549 | 0122 | 0116 |
| 3 | 2.67 | 0.14 | 0.15 | 91 | 0.45 | 0.016 | 0.025 |
| 4 | 6.53 | 0.45 | 0.31 | 92 | 0.070 | 0.0063 | 0.0075 |
| 1 | 7.00 | 0.36 | 0.65 | 99 | 0.76 | ND | 0.076 |
| 21 | 0.61 | 0.27 | 0.15 | 101 | 0.46 | 0.04 | 0.043 |
| 26 | 0.52 | 0.68 | 0.17 | 105 | 0.29 | ND | 0.085 |
| 40 | 0.23 | 0.051 | 0.060 | Brigatini | 6.13 | 0.49 | 0.43 |
| 61 | 0.28 | 0.054 | 0.040 | Avitinib | 1.06 | 1.50 | 0.97 |
| 60 | 0.23 | 0.088 | 0.038 | Osimertinib | 3.01 | 2.95 | 1.74 |
| 81 | 0.92 | ND | 0.077 | | | | |

The compound of the present invention has a very good inhibitory effect on the cell proliferation of a KC-0122:Ba/F3^{EGFR-L858R/T790M/C797S} three-mutation cell line and a KC-0116:Ba/F3^{EGFR-Del19/T790M/C797S} three-mutation cell line, and a weaker inhibitory effect on an EGFR wild-type cell line A549 with good selectivity.

### Example 120 In-Vivo Drug Efficacy Study

### 1 Materials

### 1.1 Animals

Female SPF NPG mice weighed 19-22 g were used. The experimental animals were provided by Beijing Vitalstar Biotechnology Co., Ltd. with the experimental animal production license number SCXK (Jing) 2019-0002 and the experimental animal quality certification number of No.110341211100087928.

### 1.2 Main Reagents and Instruments

### Name of Reagent:

RPMI1640, manufacturer: gibco, and lot number: 2120614; and
FBS, manufacturer: gibco, and lot number: 2094468CP;

### Name of Instrument:

carbon dioxide incubator, instrument manufacturer: Thermo, and model: BB15;
clean bench, instrument manufacturer: Suzhou Antai Airtech Co., Ltd., and model: SW-CJ-2FD;
centrifuge, instrument manufacturer: Shanghai Anting Scientific Instrument Factory, and model: TDL-5A;
inverted microscope, instrument manufacturer: OLYMPUS, and model: CKX31;
balance, instrument manufacturer: Mettler, and model: PL2002; and
vernier caliper, instrument manufacturer: SATA, and model: Shida 91512.

### 2 Experimental Procedures

### 2.1 Cell Culture and Inoculation

KC-1474 cells used in the experiment were cultured in a 5% CO₂ 37° C. incubator with an RPMI1640 medium supplemented with 10% FBS and 0.5 g/mL of puromycin. Before the cells were continuously cultured for ten passages, the KC-1474 cells were adjusted to a concentration of 1×10⁸/mL, and mixed with Matrigel at a volume ratio of 1:1, and the mixture was inoculated subcutaneously into the right lateral thorax of the NPG mice at 0.1 mL/mouse with 5×10⁶ cells per mouse.

### 2.2 Grouping and Administration

When the mean tumor volume of the mice reached about 100-130 mm³, according to the tumor volume and weight, all the mice were randomly divided into six group respectively: a model group (V), an example 60 compound oral administration group (po), an example 60 compound intravenous injection low-dose group (L), and an example 60 compound intravenous injection medium-dose group (M). The mice in all the groups were administrated once daily. The administration started on the day of the grouping and continued for 39 days. The mice in the model group were intravenously injected with the same volume of a solvent. The solvent was prepared by adding 2 g of polyethylene glycol-15 hydroxystearate (HS15) into 4 mL of dimethyl sulfoxide (DMSO), then adding 40 mL of purified water, and filtering an obtained mixture by using a 0.22-μm sterilizing filter. At the end of the experiment or the humane endpoint, the animals were euthanized using sodium pentobarbital for anesthesia.

Specific grouping and administration regimens were shown in the following table:

| Code | of Test | Administration | Administration | Administration |
|---|---|---|---|---|
| group | substance | dose (mg/kg) | frequency | route |
| V | Solvent | — | qd | iv |
| po | Example | 20 | qd | po |
| | 60 | | | |
| L | Example | 3 | qd | iv |
| | 60 | | | |
| M | Example | 10 | qd | iv |
| | 60 | | | |

### 2.3 Detection Indicators

### 2.3.1 Tumor Volume and Tumor Volume Inhibition Rate (TGI_{TV})

Tumor volume was measured once during the grouping, once per week after the grouping using a vernier caliper, and before the euthanasia. A method for measuring the tumor volume was measuring the long diameter and the short diameter of a tumor using the vernier caliper.

The tumor volume was calculated by the formula: tumor volume=0.5×long diameter×short diameter².

The tumor volume inhibition rate was calculated by the formula: TGI_{TV} (%)=[1−(Ti−T0)/(Vi−V0)]×100%

(Ti: mean tumor volume of mice in the treatment groups on day i of the administration and T0: mean tumor volume of the mice in the treatment group on day 0 of the administration; and Vi: mean tumor volume of the mice in the model group on day i of the administration and V0: mean tumor volume of the mice in the model group on day 0 of the administration).

### 2.3.2 Tumor Weight and Tumor Weight Inhibition Rate (TGI_{TW})

At the end of the experiment, the tumor tissue was stripped after the animals were euthanized, the tumor weight was weighed, and the tumor weight inhibition rate was calculated according to the following formula:

TGI_{TW}(%)=(W_{model group}−W_{treatment group})/W_{model group}×100%, wherein the W means tumor weight.

### 3 Results and Conclusions

Original data was analyzed and the results were expressed as mean±standard deviation (mean±SD). At the same time, the tumor volume and the tumor weight were subjected to statistical analysis. AP<0.05 was considered to be a significant difference.

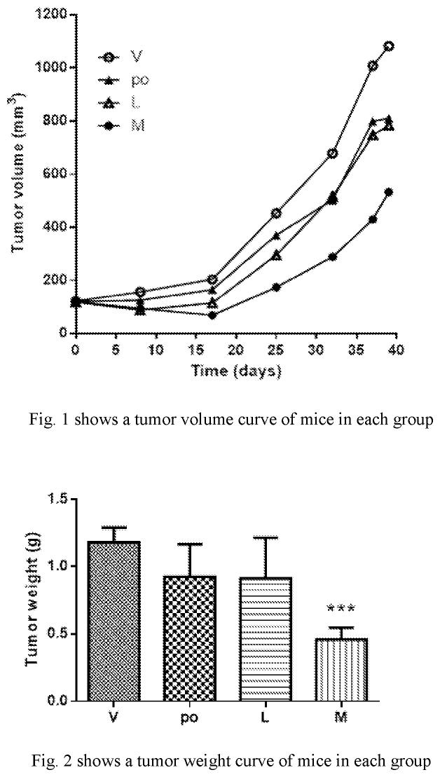

All the animals were well mobile and fed during the administration and observation with no diarrhea, vomiting, or rash. As shown in FIG. 1, at the end of the experiment, the mean tumor volume of the mice in the model group was 1,082±225 mm³, and the tumor volume inhibition rates of the mice in the po, L, and M groups were 28%, 31%, and 57%, respectively. As shown in FIG. 2, the mean tumor weight of the mice in the model group was 1.1792±0.0989 g, and the tumor weight inhibition rates of the mice in the po, L, and M groups were 21%, 22%, and 61%, respectively.

In conclusion, in the mouse model of KC-1474 cell subcutaneous transplantation tumor, compared with the model group, the growth of tumor was inhibited in the po, L, and M groups, wherein the difference between the M group and the model group was extremely significant (P <0.001). Besides, the tumor volume was reduced in the po, L, and M groups, showing a good antitumor drug efficacy.

1. A compound shown in formula I, a stereoisomer thereof, or a pharmaceutically acceptable salt thereof, 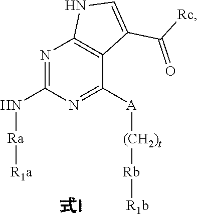
wherein the Rc is or 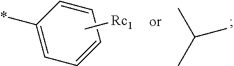
the number of the Rc₁ is 0, 1, 2, or 3; each Rc₁ is independently selected from at least one of halogen and C₁₋₃ alkyl;
the Ra is a substituted or unsubstituted phenyl, or a substituted or unsubstituted 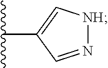 [SMILES: *c1cn[nH]c1]
the substituents are each independently selected from halogen, C₁₋₃ alkyl, and C₁₋₃ alkoxy; the number of the respective substituents of the phenyl or the 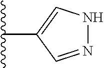 [SMILES: *c1cn[nH]c1]
is 0, 1, or 2; the halogen is F, Cl, Br, and I;
the R₁a is selected from H, 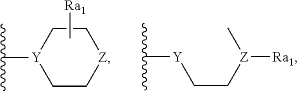
or C₁₋₃ alkyl; the Y and the Z are each independently selected from N, O, and C, wherein the N and the C are substituted by 0 or 1 C₁₋₃ alkyl and the insufficient valence bonds are complemented by H; and the Ra₁ is H or C₁₋₃ alkyl;
the A is O or N, wherein the N is substituted by at least one of H and C₁₋₃ alkyl;
the t is selected from 0, 1, 2, or 3;
the Rb is selected from groups in one or more groups of the following 1)-5):
1) a substituted or unsubstituted 5- to 8-membered spiroheterocycle, wherein a heteroatom in the spiroheterocycle is O, S, or N; the substituent of the spiroheterocycle is one or more independent substituted or unsubstituted amino, C₁₋₃ alkyl, C₁₋₃ alkoxy, and OH; and the substituent of the amino is H or one or two C₁₋₃ alkyls;
2) a substituted or unsubstituted C₈-C₁₁ bridge ring, wherein the substituent of the bridge ring is one or more independent substituted or unsubstituted amino, C₁₋₃ alkyl, C₁₋₃ alkoxy, and OH; and the substituent of the amino is H or one or two C₁₋₃ alkyls;
3) a substituted or unsubstituted amino, wherein the substituent of the amino is H or one or two C₁₋₃ alkyls, or —(CH₂)ₐ-E-(CH₂)_{b}—, and N in the amino is linked with both ends of the —(CH₂)ₐ-E-(CH₂)_{b}— to form a ring; the E is C or O, and the a is 1, 2, 3, or 4; and the b is 1, 2, 3, or 4;
4) 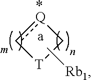
wherein the number of the double bonds a is 0, 1, 2, or 3; the position of the a is any position with a reasonable valence bond in the ring; the insufficient valence bonds are complemented by H; the m is 0, 1, 2, or 3; the n is 0, 1, 2, or 3; the Q and the T are each independently N, O, S, or C; and the insufficient valence bonds are complemented by H; and
5) 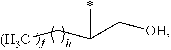
wherein the f is 1, 2, or 3; and the h is 0, 1, or 2;
the number of the Rb₁ is 0, 1, or 2; the Rb₁ is amino, hydroxy, C₁₋₃ alkyl, C₁₋₃ alkoxy, C₁₋₃ alkyl substituted by hydroxy, SO₂R₂b 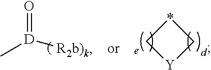
the R₂b is a substituted or unsubstituted C₁₋₃ alkyl, a substituted or unsubstituted phenyl, C₃₋₆ cycloalkyl, C₂₋₅ alkenyl, or 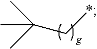
the substituent of the C₁₋₃ alkyl or the phenyl is halogen or hydroxyl; the D is selected from C, P, or S; the g is 1, 2, or 3; the e and the d are each independently selected from 0, 1, 2, or 3; and the Y is selected from C, O, or N;
the R₁b is H, NH₂, hydroxyl, C₁₋₃ alkoxy, C₁₋₃ alkyl substituted by hydroxyl, SO₂R₂b, C₁₋₃ alkyl, 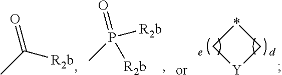
the R₂b is independently a substituted or unsubstituted C₁₋₃ alkyl, a substituted or unsubstituted phenyl, C₃₋₆ cycloalkyl, C₂₋₅ alkenyl, or 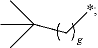
the substituent of the C₁₋₃ alkyl or the phenyl is halogen or hydroxyl; the g is 1, 2, or 3; the e and the d are each independently selected from 0, 1, 2, or 3; and the Y is selected from C, O, or N; and
the C₁₋₃ alkyl is methyl, ethyl, propyl, or isopropyl; the C₁₋₃ alkoxy is methoxy, ethoxy, propoxy, or isopropoxy; and the halogen is F, Cl, Br, or I.

2. The compound, the stereoisomer thereof, or the pharmaceutically acceptable salt thereof according to claim 1, wherein the Rc is 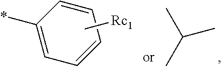
the number of the Rc₁ is 0, 1, 2, or 3; each Rc₁ is independently selected from at least one of F, Cl, Br, I, and C1-3 alkyl;
the Ra is a substituted or unsubstituted phenyl, or a substituted or unsubstituted 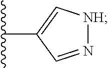 [SMILES: *c1cn[nH]c1]
the substituents are each independently selected from F, C1-3 alkyl, and C1-3 alkoxy; the number of the respective substituents of the phenyl or the 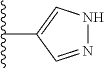 [SMILES: *c1cn[nH]c1]
is independently 0, 1, or 2;
the R₁a is selected from H 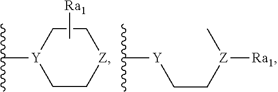
or C₁₋₃ alkyl; the Y is C or N; the Z is N or O, wherein the N and the C are substituted by 0 or 1 C₁₋₃ alkyl; and the Ra₁ is H or C₁₋₃ alkyl;
the Rb is selected from groups in one or more groups of the following 1)-5):
1) a substituted or unsubstituted 5- to 8-membered spiroheterocycle, wherein a heteroatom in the spiroheterocycle is O;
2) a substituted or unsubstituted C10 bridge ring, wherein the substituent of the bridge ring is one or more independent amino, C1-3 alkyl, C1-3 alkoxy, and OH;
3) a substituted or unsubstituted amino, wherein the substituent of the amino is one or two C₁₋₃ alkyls, or —(CH₂)ₐ-E-(CH₂)_{b}—, and N in the amino is linked with both ends of the —(CH₂)ₐ-E-(CH₂)_{b}— to form a ring; the E is C or O, and the a is 1, 2, or 3; and the b is 1, 2, or 3;
4) 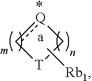
wherein the number of the double bonds a is 0, 1, 2, or 3; the position of the a is any position with a reasonable valence bond in the ring; the m is 0, 1, 2, or 3; the n is 0, 1, 2, or 3; the Q and the T are each independently N, O, S, or C; and the insufficient valence bonds are complemented by H; and
5) 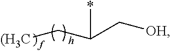
wherein the f is 1, 2, or 3; and the h is 0 or 1;
the number of the Rb₁ is 0, 1, or 2; the Rb₁ is amino, hydroxy, C₁₋₃ alkyl, C₁₋₃ alkoxy or C₁₋₃ alkyl substituted by hydroxy, SO₂R₂b, 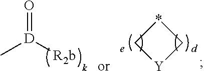
each R₂b is independently a substituted or unsubstituted C₁₋₃ alkyl, a substituted or unsubstituted phenyl, C₃₋₆ cycloalkyl, C₂₋₅ alkenyl, or 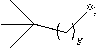
the substituent of the C₁₋₃ alkyl or the phenyl is halogen or hydroxyl; the D is selected from C, P, or S; the k is 1 or 2; the g is 1, 2, or 3; the e and the d are each independently selected from 0, 1, 2, or 3; and Y is selected from C, O, or N; and
the R₁b is H, NH₂, hydroxyl, C₁₋₃ alkoxy, C₁₋₃ alkyl substituted by hydroxyl, SO₂R₂b, C₁₋₃ alkyl, 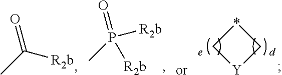
each R₂b is independently a substituted or unsubstituted C₁₋₃ alkyl, a substituted or unsubstituted phenyl, C₃₋₆ cycloalkyl, C₂₋₅ alkenyl, or 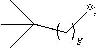
the substituent of the C₁₋₃ alkyl or the phenyl is halogen or hydroxyl; the g is 1, 2, or 3; the e and the d are each independently selected from 0, 1, 2, or 3; and the Y is selected from C, O, or N.

3. The compound, the stereoisomer thereof, or the pharmaceutically acceptable salt thereof according to claim 1, wherein the Rc is 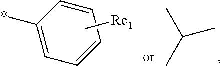
the number of the Rc₁ is 0, 1, 2, or 3; each Rc₁ is independently selected from at least one of F, Cl, Br, I, and C₁₋₃ alkyl the Ra is a substituted or unsubstituted phenyl, or a substituted or unsubstituted 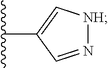 [SMILES: *c1cn[nH]c1]
the substituents are each independently selected from F, C1-3 alkyl, and C1-3 alkoxy; the number of the respective substituents of the phenyl or the 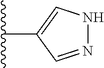 [SMILES: *c1cn[nH]c1]
is independently 0, 1, or 2;
the R₁a is selected from H, 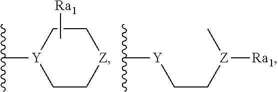
or C₁₋₃ alkyl; the Y is C or N; the Z is N or O and the Ra₁ is H or C₁₋₃ alkyl;
the Rb is selected from groups in one or more groups of the following 1)-5):
1) a substituted or unsubstituted 5- to 8-membered spiroheterocycle, wherein a heteroatom in the spiroheterocycle is O;
2) a substituted or unsubstituted C10 bridge ring, wherein the substituent of the bridge ring is one or more independent amino, C₁₋₃ alkyl, C₁₋₃ alkoxy, and OH;
3) a substituted or unsubstituted amino, wherein the substituent of the amino is one or two C₁₋₃ alkyls, or —(CH₂)ₐ-E-(CH₂)_{b}—, and N in the amino is linked with both ends of the —(CH₂)ₐ-E-(CH₂)_{b}— to form a ring; the E is C or O, and the a is 1, 2, or 3; and the b is 1, 2, or 3;
4) 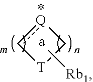
wherein the number of the double bonds a is 0, 1, 2, or 3; the position of the a is any position with a reasonable valence bond in the ring; the insufficient valence bonds are complemented by H; the m is 0, 1, 2, or 3; the n is 0, 1, 2, or 3; the Q and the T are each independently N, O, S, or C; and the insufficient valence bonds are complemented by H; and
5) 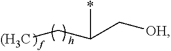
wherein the f is 1, 2, or 3; and the h is 0 or 1;
the number of the Rb₁ is 0, 1, or 2; the Rb₁ is amino, hydroxy, C₁₋₃ alkyl, C₁₋₃ alkoxy or C₁₋₃ alkyl substituted by hydroxy, SO₂R₂b, 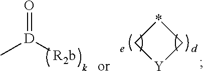
each R₂b is independently a substituted or unsubstituted C₁₋₃ alkyl, a substituted or unsubstituted phenyl, C₃₋₆ cycloalkyl, C₂₋₅ alkenyl, or 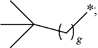
the substituent of the C₁₋₃ alkyl or the phenyl is halogen or hydroxyl; the D is selected from C, P, or S; the k is 1 or 2; the g is 1, 2, or 3; the e and the d are each independently selected from 0, 1, 2, or 3; and Y is selected from C, O, or N; and
the R₁b is H, NH₂, hydroxyl, C₁₋₃ alkoxy, C₁₋₃ alkyl substituted by hydroxyl, SO₂R₂b, C₁₋₃ alkyl, 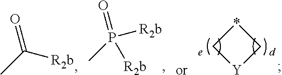
each R₂b is independently a substituted or unsubstituted C₁₋₃ alkyl, a substituted or unsubstituted phenyl, C₃₋₆ cycloalkyl, C₂₋₅ alkenyl, or 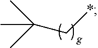
the substituent of the C₁₋₃ alkyl or the phenyl is halogen or hydroxyl; the g is 1, 2, or 3; the e and the d are each independently selected from 0, 1, 2, or 3; and the Y is selected from C, O, or N.

4. The compound, the stereoisomer thereof, or the pharmaceutically acceptable salt thereof according to claim 1, wherein the Rc is or 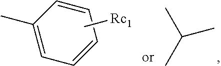
the number of the Rc₁ is 0, 1, 2, or 3; each Rc1 is independently selected from at least one of F, Cl, and C₁₋₃ alkyl;
the Ra is a substituted or unsubstituted phenyl, or a substituted or unsubstituted 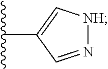 [SMILES: *c1cn[nH]c1]
the substituent of the phenyl or the 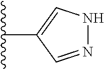 [SMILES: *c1cn[nH]c1]
in the Ra is F, methyl, and methoxy; and the number of the respective substituents of the phenyl or the 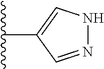 [SMILES: *c1cn[nH]c1]
is 1;
the R₁a is selected from H, 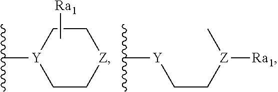
or C₁₋₃ alkyl; the Y is C or N; the Z is N or O and the Ra₁ is H, CH₃, or CH₂CH₃; t is selected from 0, 1, or 2;
the Rb is selected from groups in one or more groups of the following 1)-5):
1) a substituted or unsubstituted 5- to 8-membered spiroheterocycle, wherein a heteroatom in the spiroheterocycle is O;
2) a substituted or unsubstituted C10 bridge ring, wherein the substituent of the bridge ring is one or more independent amino, C₁₋₃ alkyl, C₁₋₃ alkoxy, and OH;
3) a substituted or unsubstituted amino, wherein the substituent of the amino is one or two C₁₋₃ alkyls, or —(CH₂)ₐ-E-(CH₂)_{b}—, and N in the amino is linked with both ends of the —(CH₂)ₐ-E-(CH₂)_{b}— to form a ring; the E is C or O, and the a is 1, 2, or 3; and the b is 1, 2, or 3;
4) 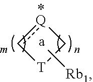
wherein the number of the double bonds a is 0, 1, 2, or 3; the position of the a is any position with a reasonable valence bond in the ring; the insufficient valence bonds are complemented by H; the m is 0, 1, 2, or 3; the n is 0, 1, 2, or 3; the Q and the T are each independently N, O, S, or C; and the insufficient valence bonds are complemented by H; and
5) 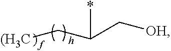
wherein the f is 1, 2, or 3; and the h is 0 or 1;
the number of the Rb₁ is 0 or 1; the Rb₁ is amino, hydroxy, C₁₋₃ alkyl, C₁₋₃ alkoxy or C₁₋₃ alkyl substituted by hydroxy, SO₂R₂b, 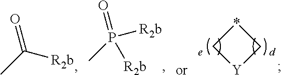
each R₂b is independently a substituted or unsubstituted C₁₋₃ alkyl, a substituted or unsubstituted phenyl, C₃₋₆ cycloalkyl, C₂₋₅ alkenyl, or 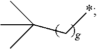
the substituent of the C₁₋₃ alkyl or the phenyl is halogen or hydroxyl; the g is 1, 2, or 3; the e and the d are each independently selected from 0, 1, 2, or 3; and the Y is selected from C, O, or N; and
the R₁b is H, NH₂, hydroxymethyl, hydroxyethylhydroxypropyl, methyl, ethyl, propyl, isopropyl, OH, SO₂R₂b, ((CH₃)₂CH₂, 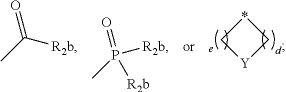
each R₂b is independently a substituted or unsubstituted C₁₋₃ alkyl, a substituted or unsubstituted phenyl, C₃₋₅ cycloalkyl, C₂₋₃ alkenyl, or 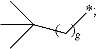
the substituent of the phenyl is halogen or hydroxyl; the g is 1, 2, or 3; the e and the d are each independently selected from 0, 1, 2, or 3; the Y is selected from C, O, or N; and the halogen is F, Cl, Br, or I.

5. The compound, the stereoisomer thereof, or the pharmaceutically acceptable salt thereof according to claim 1, wherein the Rc is 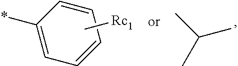
the number of the Rc₁ is 0, 1, 2, or 3; each Rc₁ is independently selected from at least one of F, Cl, and C₁₋₃ alkyl;
the Ra is a substituted or unsubstituted phenyl, or a substituted or unsubstituted 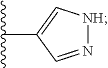 [SMILES: *c1cn[nH]c1]
each substituent of the phenyl or the 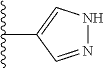 [SMILES: *c1cn[nH]c1]
in the Ra is independently selected from F, methyl, and methoxy; and the number of the respective substituents of the phenyl or the 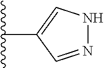 [SMILES: *c1cn[nH]c1]
is independently 0, 1, or 2;
the R₁a is selected from H, 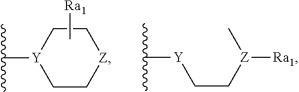
or C₁₋₃ alkyl; the Y is N, O, or C; the Z is N or O;
the Ra₁ is H, CH₃, or CH₂CH₃; A is O or N, wherein the N is substituted by an H; t is selected from 0, 1, or 2;
the Rb is selected from groups in one or more groups of the following 1)-4):
1) a substituted or unsubstituted 7-membered spiroheterocycle, wherein a heteroatom in the spiroheterocycle is O;
2) a substituted or unsubstituted adamantyl, wherein the substituent of the adamantyl is one or more OHs;
3) a substituted or unsubstituted amino, wherein the substituent of the amino is one or two C₁₋₃ alkyls; and
4) 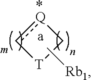
wherein the number of the double bonds a is 0 or 1; the position of the a is any position with a reasonable valence bond in the ring; the insufficient valence bonds are complemented by H; the m is 0, 1, 2, or 3; the n is 0, 1, or 2; the Q is C; the T is N, O or C; and
the insufficient valence bonds are complemented by H;
the number of the Rb₁ is 0 or 1; the Rb₁ is amino, hydroxy, C₁₋₃ alkyl, C₁₋₃ alkoxy or C₁₋₃ alkyl substituted by hydroxy, SO₂R₂b, 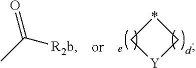
each R₂b is independently a substituted or unsubstituted C₁₋₃ alkyl, cyclopropyl, or C₂₋₃ alkenyl; the substituent of the C₁₋₃ alkyl is halogen or hydroxyl; the e and the d are each independently selected from 0, 1, 2, or 3; and the Y is selected from C, O, or N; and
the R₁b is amino, hydroxy, C₁₋₃ alkyl substituted by hydroxy, C₁₋₃ alkoxy, SO₂R₂b, or 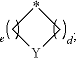
the R₂b is selected from C₁₋₃ alkyl or C₃₋₆ cycloalkyl; the e and the d are each independently selected from 1, 2, or 3; and the Y is selected from C, O, or N.

6. The compound, the stereoisomer thereof, or the pharmaceutically acceptable salt thereof according to claim 1, wherein the Rc is or 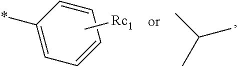
the number of the Rc₁ is 0, 1, 2, or 3; each Rc₁ is independently selected from at least one of F, Cl, and C₁₋₃ alkyl;
the Ra is a substituted or unsubstituted phenyl, or a substituted or unsubstituted 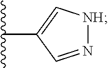 [SMILES: *c1cn[nH]c1]
the substituents are each independently selected from F, methyl, and methoxy; and the number of the respective substituents of the phenyl or the 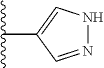 [SMILES: *c1cn[nH]c1]
is independently 0, 1, or 2;
the R₁a is selected from H, 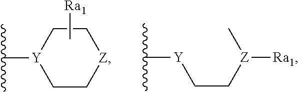
or C₁₋₃ alkyl; the Y is N, O, or C; the Z is N or O;
the Ra₁ is H, CH₃, or CH₂CH₃; A is O or N, wherein the N is substituted by an H; t is selected from 0, 1, or 2;
the Rb is selected from groups in one or more groups of the following 1)-4):
1) a substituted or unsubstituted 7-membered spiroheterocycle, wherein a heteroatom in the spiroheterocycle is O;
2) a substituted or unsubstituted adamantyl, wherein the substituent of the adamantyl is one or more OHs;
3) a substituted or unsubstituted amino, wherein the substituent of the amino is one or two C₁₋₃ alkyls; and
4) 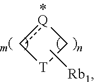
wherein the number of the double bonds a is 0 or 1; the position of the a is any position with a reasonable valence bond in the ring; the insufficient valence bonds are complemented by H; the m is 0, 1, 2, or 3; the n is 0, 1, or 2; the Q is C; the T is N, O or C; and
the insufficient valence bonds are complemented by H;
the number of the Rb₁ is 0, 1, or 2; the Rb₁ is amino, hydroxy, C₁₋₃ alkyl, C₁₋₃ alkoxy or C₁₋₃ alkyl substituted by hydroxy, SO₂R₂b, 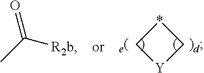
each R₂b is independently a substituted or unsubstituted C₁₋₃ alkyl, cyclopropyl, or C₂₋₃ alkenyl; the substituent of the C₁₋₃ alkyl is halogen or hydroxyl; the e and the d are each independently selected from 0, 1, 2, or 3;
and the Y is selected from C, O, or N; and
the R₁b is H, amino, hydroxy, C₁₋₃ alkyl substituted by hydroxy, C₁₋₃ alkoxy, SO₂R₂b, or 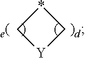
the R₂b is selected from C₁₋₃ alkyl or C₃₋₆ cycloalkyl; the e and the d are each independently selected from 1, 2, or 3; and the Y is selected from C, O, or N.

7. The compound, the stereoisomer thereof, or the pharmaceutically acceptable salt thereof according to claim 1, wherein the Rc in formula I is a substituted or unsubstituted phenyl, as shown in formula II, 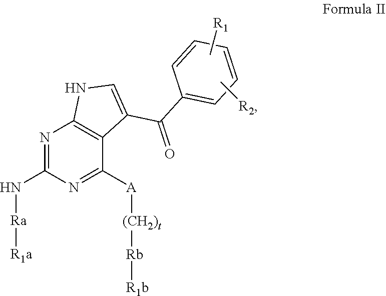
the R₁ and the R₂ are each independently selected from at least one of H, halogen, and C1-3 alkyl.

8. The compound, the stereoisomer thereof, or the pharmaceutically acceptable salt thereof according to claim 1, wherein the Rc in formula I is a substituted or unsubstituted phenyl, the Ra is a substituted or unsubstituted benzene ring, and
the Rb is 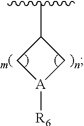
the R₁b is H, and as shown in formula III 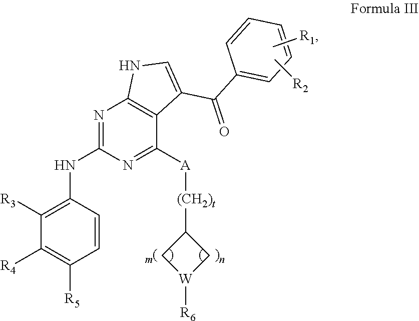
wherein the m is selected from 0, 1, or 2; the n is selected from 0, 1, or 2; the t is selected from 0, 1, or 2; the W is C, O, or N; the R₁ is selected from H, F, or Cl; the R₂ is selected from H, F, or Cl; the R₃ is selected from H or MeO; the R₄ is selected from H or F; the R₅ is selected from H, 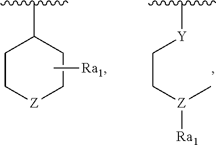
or C₁₋₃ alkyl; the Y and the Z are each independently selected from N, O, and C, and the insufficient valence bonds are complemented by H; and the Ra₁ is H or C₁₋₃ alkyl; and
R₆ is selected from H, NH₂, hydroxyl, C13 alkoxy, C₁₋₃ alkyl substituted by hydroxyl, SO₂R₂b, C₁₋₃ alkyl, 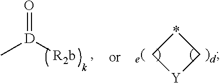
each R₂b is independently a substituted or unsubstituted C₁₋₃ alkyl, a substituted or unsubstituted phenyl, C₃₋₆ cycloalkyl, C₂₋₅ alkenyl, or 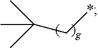
the D is selected from C, P, or S; the k is 1 or 2; the substituent of the C₁₋₃ alkyl or the phenyl is halogen or hydroxyl; the g is 1, 2, or 3; the e and the d are each independently selected from 0, 1, 2, or 3; and the Y is selected from C, O, or N.

9. The compound, the stereoisomer thereof, or the pharmaceutically acceptable salt thereof according to claim 1, wherein in formula I, the Ra is a substituted or unsubstituted 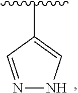 [SMILES: *c1cn[nH]c1]
and the substituent is at least one of C₁₋₃ alkoxy and C₁₋₃ alkyl; and further, the substituent is one, two or three of H, methoxy, ethoxy, propoxy, isopropoxy, methyl, ethyl, and propyl.

10. The compound, the stereoisomer thereof, or the pharmaceutically acceptable salt thereof according to claim 1, wherein in formula I, the Ra is Ra 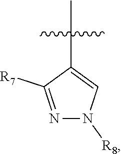
and the Rb is selected from 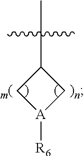
the R₁b is H, and as shown in formula IV, 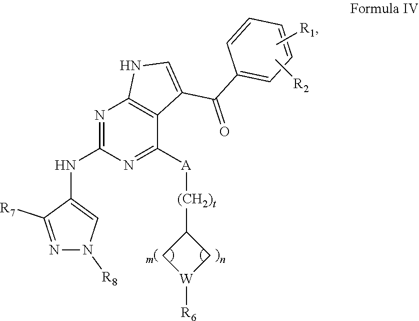
wherein the m is selected from 0, 1, or 2; the n is selected from 0, 1, or 2; the t is selected from 0, 1, or 2; the W is C, O, or N; the R₁ is selected from H, F, or Cl; the R₂ is selected from H, F, or Cl; the R₆ is selected from H, OH, CH₂OH, 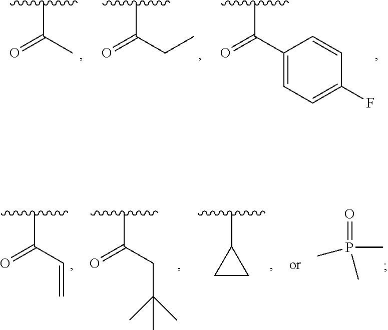
R₇ is selected from H or C₁₋₃ alkoxy; and R₈ is selected from C₁₋₃ alkyl or piperidyl substituted by C₁₋₃ alkyl.

11. The compound, the stereoisomer thereof, or the pharmaceutically acceptable salt thereof according to claim 1, wherein the compound is selected from:
(S)—N-(1-(5-(4-fluorobenzoyl)-2-((5-(4-methylpiperazin-1-yl)pyridin-2-yl)amino)-7H-pyrrolo[2,3-d]pyrimidin-4-yl)piperidin-3-yl)cyclopropanesulfonamide, (S)—N-(1-(5-(4-fluorobenzoyl)-2-((5-(4-methylpiperazin-1-yl)pyridin-2-yl)amino)-7H-pyrrolo[2,3-d]pyrimidin-4-yl)pyrrolidin-3-yl)cyclopropanesulfonamide, (S)—N-(1-(5-(4-fluorobenzoyl)-2-((6-(4-methylpiperazin-1-yl)pyridin-3-yl)amino)-7H-pyrrolo[2,3-d]pyrimidin-4-yl)pyrrolidin-3-yl)cyclopropanesulfonamide, (S)—N-(1-(5-(4-fluorobenzoyl)-2-((1-methyl-1H-pyrazol-4-yl)amino)-7H-pyrrolo[2,3-d]pyrimidin-4-yl)pyrrolidin-3-yl)cyclopropanesulfonamide, (S)—N-(1-(5-(4-fluorobenzoyl)-2-((4-(4-methylpiperazin-1-yl)phenyl)amino)-7H-pyrrolo[2,3-d]pyrimidin-4-yl)piperidin-3-yl)cyclopropanesulfonamide, (R)-(4-((1-(cyclopropylsulfonyl)piperidin-3-yl)amino)-2-((4-(4-methylpiperazin-1-yl)phenyl)amino)-7H-pyrrolo[2,3-d]pyrimidin-5-yl)(4-fluorophenyl)methanone, (R)-(4-(1-(cyclopropylsulfonyl)piperidin-3-yl)amino)-2-((1-methyl-1H-pyrazol-4-yl)amino)-7H-pyrrolo[2,3-d]pyrimidin-5-yl)(4-fluorophenyl)methanone, (R)-1-(4-((1-(cyclopropylsulfonyl)piperidin-3-yl)amino)-2-((4-(4-methylpiperazin-1-yl)phenyl)amino)-7H-pyrrolo[2,3-d]pyrimidin-5-yl)-2-methylpropan-1-one, (S)-(4-(1-(cyclopropylsulfonyl)piperidin-3-yl)amino)-2-((4-(4-methylpiperazin-1-yl)phenyl)amino)-7H-pyrrolo[2,3-d]pyrimidin-5-yl)(4-fluorophenyl)methanone, (R)-(4-(1-(cyclopropylsulfonyl)pyrrolidin-3-yl)amino)₂-((4-(4-methylpiperazin-1-yl)phenyl)amino)-7H-pyrrolo[2,3-d]pyrimidin-5-yl)(4-fluorophenyl)methanone, (S)-(4-(1-(cyclopropylsulfonyl)pyrrolidin-3-yl)amino)-2-((4-(4-methylpiperazin-1-yl)phenyl)amino)-7H-pyrrolo[2,3-d]pyrimidin-5-yl)(4-fluorophenyl)methanone, (S)-1-(3-((5-(4-fluorobenzoyl)-2-((4-(4-methylpiperazin-1-yl)phenyl)amino)-7H-pyrrolo[2,3-d]pyrimidin-4-yl)amino)piperidin-1-yl)prop-2-en-1-one, (S)-1-(3-((5-(4-fluorobenzoyl)-2-((4-(4-methylpiperazin-1-yl)phenyl)amino)-7H-pyrrolo[2,3-d]pyrimidin-4-yl)amino)pyrrolidin-1-yl)prop-2-en-1-one, (S)-(4-(1-(cyclopropylsulfonyl)piperidin-3-yl)amino)-2-((4-(4-ethylpiperazin-1-yl)phenyl)amino)-7H-pyrrolo[2,3-d]pyrimidin-5-yl)(4-fluorophenyl)methanone, (S)-(4-(1-(cyclopropylsulfonyl)pyrrolidin-3-yl)amino)-2-((4-(4-methylpiperazin-1-yl)phenyl)amino)-7H-pyrrolo[2,3-d]pyrimidin-5-yl)(phenyl)methanone, (S)-(4-(1-(cyclopropylsulfonyl)pyrrolidin-3-yl)amino)-2-((4-(4-methylpiperazin-1-yl)phenyl)amino)-7H-pyrrolo[2,3-d]pyrimidin-5-yl)(3-fluorophenyl)methanone, (S)-(4-(1-(cyclopropylsulfonyl)pyrrolidin-3-yl)amino)-2-((4-(4-methylpiperazin-1-yl)phenyl)amino)-7H-pyrrolo[2,3-d]pyrimidin-5-yl)(3,4-difluorophenyl)methanone, (4-fluorophenyl)(2-((4-(4-methylpiperazin-1-yl)phenyl)amino)-4-(morpholinoamino)-7H-pyrrolo[2,3-d]pyrimidin-5-yl)methanone, (4-fluorophenyl)(2-((4-(4-methylpiperazin-1-yl)phenyl)amino)-4-((tetrahydro-2H-pyran-4-yl)amino)-7H-pyrrolo[2,3-d]pyrimidin-5-yl)methanone, (4-fluorophenyl)(4-(((1r,4r)-4-(hydroxymethyl)cyclohexyl)amino)-2-((4-(4-methylpiperazin-1-yl)phenyl)amino)-7H-pyrrolo[2,3-d]pyrimidin-5-yl)methanone, (S)-1-(3-((5-(2,4-difluorobenzoyl)-2-((4-(4-methylpiperazin-1-yl)phenyl)amino)-7H-pyrrolo[2,3-d]pyrimidin-4-yl)amino)pyrrolidin-1-yl)prop-2-en-1-one, (S)-1-(3-((5-(2-fluorobenzoyl)-2-((4-(4-methylpiperazin-1-yl)phenyl)amino)-7H-pyrrolo[2,3-d]pyrimidin-4-yl)amino)pyrrolidin-1-yl)propan-2-en-1-one, (R)-1-(3-((5-(4-fluorobenzoyl)-2-((1-methyl-1H-pyrazol-4-yl)amino)-7H-pyrrolo[2,3-d]pyrimidin-4-yl)amino)pyrrolidin-1-yl)propan-2-en-1-one, (S)-(4-((1-(cyclopropylsulfonyl)pyrrolidin-3-yl)amino)-2-((4-(4-methylpiperazin-1-yl)phenyl)amino)-7H-pyrrolo[2,3-d]pyrimidin-5-yl)(2,4-difluorophenyl)methanone, (R)-(4-fluorophenyl)(2-((4-(4-methylpiperazin-1-yl)phenyl)amino)-4-(1-(methylsulfonyl)pyrrolidin-3-yl)amino)-7H-pyrrolo[2,3-d]pyrimidin-5-yl)methanone, (S)-1-(3-((5-(4-fluorobenzoyl)-2-((4-(4-methylpiperazin-1-yl)phenyl)amino)-7H-pyrrolo[2,3-d]pyrimidin-4-yl)amino)pyrrolidin-1-yl)-3,3-dimethylbut-1-one, (S)-1-(3-((5-(4-fluorobenzoyl)-2-((4-(4-methylpiperazin-1-yl)phenyl)amino)-7H-pyrrolo[2,3-d]pyrimidin-4-yl)amino)pyrrolidin-1-yl)-2, 2-dimethylpropan-1-one, (4-fluorophenyl)(4-(((1s,4s)-4-hydroxycyclohexyl)amino)-2-((4-(4-methylpiperazin-1-yl)phenyl)amino)-7H-pyrrolo[2,3-d]pyrimidin-5-yl)methanone, (S)-(4-fluorophenyl)(2-((4-(4-methylpiperazin-1-yl)phenyl)amino)-4-(tetrahydro-2H-pyran-3-yl)amino)-7H-pyrrolo[2,3-d]pyrimidin-5-yl)methanone, (4-fluorophenyl)(2-((4-(4-methylpiperazin-1-yl)phenyl)amino)-4-((tetrahydro-2H-pyran-4-yl)methyl)amino)-7H-pyrrolo[2,3-d]pyrimidin-5-yl)methanone, (4-fluorophenyl)(4-(methyl(tetrahydro-2H-pyran-4-yl)amino)-2-(4-((4-methylpiperazin-1-yl)phenyl)amino)-7H-pyrrolo[2,3-d]pyrimidin-5-yl)methanone, 1-(4-((5-(4-fluorobenzoyl)-2-((4-(4-methylpiperazin-1-yl)phenyl)amino)-7H-pyrrolo[2,3-d]pyrimidin-4-yl)amino)piperidin-1-yl)prop-2-en-1-one, (4-fluorophenyl)(2-((4-(4-methylpiperazin-1-yl)phenyl)amino)-4-((1-(methylsulfonyl)piperidin-4-yl)amino)-7H-pyrrolo[2,3-d]pyrimidin-5-yl)methanone, 1-(4-((5-(4-fluorobenzoyl)-2-((4-(4-methylpiperazin-1-yl)phenyl)amino)-7H-pyrrolo[2,3-d]pyrimidin-4-yl)amino)piperidin-1-yl)-3,3-dimethylbut-1-one, (R)-(4-fluorophenyl)(2-((4-(4-methylpiperazin-1-yl)phenyl)amino)-4-(tetrahydrofuran-3-yl)amino)-7H-pyrrolo[2,3-d]pyrimidin-5-yl)methanone, (4-fluorophenyl)(4-((3-methyloxetan-3-yl)amino)-2-((4-(4-methylpiperazin-1-yl)phenyl)amino)-7H-pyrrolo[2,3-d]pyrimidin-5-yl)methanone, (4-fluorophenyl)(4-(((1s,3R,4s,5S,7s)-4-hydroxyadamantan-1-yl)amino)-2-((4-(4-methylpiperazin-1-yl)phenyl)amino)-7H-pyrrolo[2,3-d]pyrimidin-5-yl)methanone, (R)-(4-fluorophenyl)(4-((1-hydroxy-4-methylpentan-2-yl)amino)-2-((4-(4-methylpiperazin-1-yl)phenyl)amino)-7H-pyrrolo[2,3-d]pyrimidin-5-yl)methanone, (S)-(4-fluorophenyl)(4-((1-hydroxy-3,3-dimethylbut-2-yl)amino)-2-((4-(4-methylpiperazin-1-yl)phenyl)amino)-7H-pyrrolo[2,3-d]pyrimidin-5-yl)methanone, (4-fluorophenyl)(2-((4-(4-methylpiperazin-1-yl)phenyl)amino)-4-((2-(tetrahydro-2H-pyran-4-yl)ethyl)amino)-7H-pyrrolo[2,3-d]pyrimidin-5-yl)methanone, (4-fluorophenyl)(4-((1-(hydroxymethyl)cyclopentyl)amino)-2-((4-(4-methylpiperazin-1-yl)phenyl)amino)-7H-pyrrolo[2,3-d]pyrimidin-5-yl)methanone, (4-fluorophenyl)(2-((4-(4-methylpiperazin-1-yl)phenyl)amino)-4-((tetrahydro-2H-pyran-3-yl)amino)-7H-pyrrolo[2,3-d]pyrimidin-5-yl)methanone, (4-((2-(diethylamino)ethyl)amino)-2-((4-(4-methylpiperazin-1-yl)phenyl)amino)-7H-pyrrolo[2,3-d]pyrimidin-5-yl)(4-fluorophenyl)methanone, (4-fluorophenyl)(2-((4-(4-methylpiperazin-1-yl)phenyl)amino)-4-((2-(pyrrolidin-1-yl)ethyl)amino)-7H-pyrrolo[2,3-d]pyrimidin-5-yl)methanone, (4-fluorophenyl)(4-((4-hydroxytetrahydro-2H-pyran-4-yl)methyl)amino)-2-((4-(4-methylpiperazin-1-yl)phenyl)amino)-7H-pyrrolo[2,3-d]pyrimidin-5-yl)methanone, (4-fluorophenyl)(4-(((1r,4r)-4-(hydroxymethyl)cyclohexyl)amino)-2-((4-morpholinophenyl)amino)-7H-pyrrolo[2,3-d]pyrimidin-5-yl)methanone, (4-fluorophenyl)(4-(((1r,4r)-4-(hydroxymethyl)cyclohexyl)amino)-2-((2-methoxy-4-morpholinophenyl)amino)-7H-pyrrolo[2,3-d]pyrimidin-5-yl)methanone, (4-fluorophenyl)(4-(((1r,4r)-4-(hydroxymethyl)cyclohexyl)amino)-2-((1-methyl-1H-pyrazol-4-yl)amino)-7H-pyrrolo[2,3-d]pyrimidin-5-yl)methanone, (4-fluorophenyl)(2-((4-(4-methylpiperazin-1-yl)phenyl)amino)-4-((tetrahydro-2H-pyran-4-yl)oxy)-7H-pyrrolo[2,3-d]pyrimidin-5-yl)methanone, (4-(((1s,4s)-4-aminocyclohexyl)amino)-2-((4-(4-methylpiperazin-1-yl)phenyl)amino)-7H-pyrrolo[2,3-d]pyrimidin-5-yl)(4-fluorophenyl)methanone, (4-fluorophenyl)(4-((6-(hydroxymethyl)pyridin-3-yl)amino)-2-((4-(4-methylpiperazin-1-yl)phenyl)amino)-7H-pyrrolo[2,3-d]pyrimidin-5-yl)methanone, ((1r,4r)-4-((2-((4-morpholinophenyl)amino)-7H-pyrrolo[2,3-d]pyrimidin-4-yl)amino)cyclohexyl)methanol, (4-fluorophenyl)(4-((1-isopropylpiperidin-4-yl)amino)-2-((4-(4-methylpiperazin-1-yl)phenyl)amino)-7H-pyrrolo[2,3-d]pyrimidin-5-yl)methanone, (4-((3,5-bis(hydroxymethyl)phenyl)amino)-2-((4-(4-methylpiperazin-1-yl)phenyl)amino)-7H-pyrrolo[2,3-d]pyrimidin-5-yl)(4-fluorophenyl)methanone, (4-((2-((4-(4-methylpiperazin-1-yl)phenyl)amino)-7H-pyrrolo[2,3-d]pyrimidin-4-yl)amino)piperidin-1-yl)methanol, (3-fluorophenyl)(4-(((1r,4r)-4-(hydroxymethyl)cyclohexyl)amino)-2-((4-morpholinophenyl)amino)-7H-pyrrolo[2,3-d]pyrimidin-5-yl)methanone, (4-fluorophenyl)(4-((3R,6S)-6-(hydroxymethyl)tetrahydro-2H-pyran-3-yl)amino)-2-((4-morpholinophenyl)amino)-7H-pyrrolo[2,3-d]pyrimidin-5-yl)methanone, (2,4-difluorophenyl)(4-(((1r,4r)-4-(hydroxymethyl)cyclohexyl)amino)-2-((4-morpholinophenyl)amino)-7H-pyrrolo[2,3-d]pyrimidin-5-yl)methanone, (4-fluorophenyl)(4-(((1r,4r)-4-(hydroxymethyl)cyclohexyl)amino)-2-((3-methoxy-1-(1-methylpiperidin-4-yl)-1H-pyrazol-4-yl)amino)-7H-pyrrolo[2,3-d]pyrimidin-5-yl)methanone, (4-((2-oxaspiro[3.3]hept-6-yl)amino)-2-((4-(4-methylpiperazin-1-yl)phenyl)amino)-7H-pyrrolo[2,3-d]pyrimidin-5-yl)(4-fluorophenyl)methanone, ((1r,4r)-4-((2-((1-methyl-1H-pyrazol-4-yl)amino)-7H-pyrrolo[2,3-d]pyrimidin-4-yl)amino)cyclohexyl)methanol, (4-fluorophenyl)(4-((1-isopropylpiperidin-4-yl)amino)-2-((4-morpholinophenyl)amino)-7H-pyrrolo[2,3-d]pyrimidin-5-yl)methanone, (3,5-difluorophenyl)(4-(((1r,4r)-4-(hydroxymethyl)cyclohexyl)amino)-2-((4-morpholinophenyl)amino)-7H-pyrrolo[2,3-d]pyrimidin-5-yl)methanone, (2-((3-fluoro-4-(4-methylpiperazin-1-yl)phenyl)amino)-4-(((1s,4s)-4-(hydroxymethyl)cyclohexyl)amino)-7H-pyrrolo[2,3-d]pyrimidin-5-yl)(4-fluorophenyl)methanone, (4-fluorophenyl)(4-(((1s,4s)-4-(hydroxymethyl)cyclohexyl)amino)-2-((1-methyl-1H-pyrazol-4-yl)amino)-7H-pyrrolo[2,3-d]pyrimidin-5-yl)methanone, (4-fluorophenyl)(4-(((1s,4s)-4-(hydroxymethyl)cyclohexyl)amino)-2-((2-methoxy-4-(4-methylpiperazin-1-yl)phenyl)amino)-7H-pyrrolo[2,3-d]pyrimidin-5-yl) methanone, (2-((4-(2-(dimethylamino)ethoxy)phenyl)amino)-4-(((1s,4s)-4-(hydroxymethyl)cyclohexyl)amino)-7H-pyrrolo[2,3-d]pyrimidin-5-yl)(4-fluorophenyl)methanone, (4-fluorophenyl)(4-((1S,4R)-4-(hydroxymethyl)cyclopent-2-en-1-yl)amino)-2-((4-(4-methylpiperazin-1-yl)phenyl)amino)-7H-pyrrolo[2,3-d]pyrimidin-5-yl)methanone, (4-(((1r,4r)-4-(hydroxymethyl)cyclohexyl)amino)-2-((4-morpholinophenyl)amino)-7H-pyrrolo[2,3-d]pyrimidin-5-yl)(2,3,4-trifluorophenyl)methanone, (R)-1-(3-((5-(4-fluorobenzoyl)-2-((4-(4-methylpiperazin-1-yl)phenyl)amino)-7H-pyrrolo[2,3-d]pyrimidin-4-yl)amino)piperidin-1-yl)prop-2-en-1-one, (R)-1-(3-((5-(4-fluorobenzoyl)-2-((4-(4-methylpiperazin-1-yl)phenyl)amino)-7H-pyrrolo[2,3-d]pyrimidin-4-yl)amino)piperidin-1-yl)prop-2-en-1-one, (S)-(4-(1-(cyclopropylsulfonyl)piperidin-3-yl)amino)-2-((3-fluoro-4-(4-methylpiperazin-1-yl)phenyl)amino)-7H-pyrrolo[2,3-d]pyrimidin-5-yl)(4-fluorophenyl)methanone, (S)-(4-(1-(cyclopropylsulfonyl)piperidin-3-yl)amino)-2-((4-morpholinophenyl)amino)-7H-pyrrolo[2,3-d]pyrimidin-5-yl)(4-fluorophenyl)methanone, (S)-(4-(1-(cyclopropylsulfonyl)pyrrolidin-3-yl)amino)-2-((4-morpholinophenyl)amino)-7H-pyrrolo[2,3-d]pyrimidin-5-yl)(4-fluorophenyl)methanone, (S)-1-(3-((5-benzoyl-2-((4-(4-methylpiperazin-1-yl)phenyl)amino)-7H-pyrrolo[2,3-d]pyrimidin-4-yl)amino)pyrrolidin-1-yl)prop-2-en-1-one, (S)-1-(3-((5-(3-fluorobenzoyl)-2-((4-(4-methylpiperazin-1-yl)phenyl)amino)-7H-pyrrolo[2,3-d]pyrimidin-4-yl)amino)pyrrolidin-1-yl)prop-2-en-1-one, (S)-1-(3-((5-(3,4-difluorobenzoyl)-2-((4-(4-methylpiperazin-1-yl)phenyl)amino)-7H-pyrrolo[2,3-d]pyrimidin-4-yl)amino)pyrrolidin-1-yl)prop-2-en-1-one, (4-fluorophenyl)(2-((4-(4-methylpiperazin-1-yl)phenyl)amino)-4-(piperidin-1-ylamino)-7H-pyrrolo[2,3-d]pyrimidin-5-yl)methanone, (4-(cyclohexylamino)-2-((4-(4-methylpiperazin-1-yl)phenyl)amino)-7H-pyrrolo[2,3-d]pyrimidin-5-yl)(4-fluorophenyl)methanone, (4-fluorophenyl)(4-((2-hydroxycyclohexyl)amino)-2-((4-(4-methylpiperazin-1-yl)phenyl)amino)-7H-pyrrolo[2,3-d]pyrimidin-5-yl)methanone, (S)-(4-((1-(cyclopropylsulfonyl)pyrrolidin-3-yl)amino)-2-((4-(4-methylpiperazin-1-yl)phenyl)amino)-7H-pyrrolo[2,3-d]pyrimidin-5-yl)(2-fluorophenyl)methanone, (4-((2-(dimethylphosphoryl)phenyl)amino)-2-((4-(4-methylpiperazin-1-yl)phenyl)amino)-7H-pyrrolo[2,3-d]pyrimidin-5-yl)(4-fluorophenyl)methanone, (R)-(4-fluorophenyl)(2-((4-(4-methylpiperazin-1-yl)phenyl)amino)-4-(1-(methylsulfonyl)piperidin-3-yl)amino)-7H-pyrrolo[2,3-d]pyrimidin-5-yl)methanone, (S)-(3-((5-(4-fluorobenzoyl)-2-(4-(4-methylpiperazin-1-yl)phenyl)amino)-7H-pyrrolo[2,3-d]pyrimidin-4-yl)amino)pyrrolidin-1-yl)(4-fluorophenyl)methanone, (S)-1-(3-((5-(4-fluorobenzoyl)-2-((4-(4-methylpiperazin-1-yl)phenyl)amino)-7H-pyrrolo[2,3-d]pyrimidin-4-yl)amino)pyrrolidin-1-yl)ethyl-1-one, (4-fluorophenyl)(4-(((1r,4r)-4-hydroxycyclohexyl)amino)-2-((4-(4-methylpiperazin-1-yl)phenyl)amino)-7H-pyrrolo[2,3-d]pyrimidin-5-yl)methanone, (R)-(4-fluorophenyl)(2-((4-(4-methylpiperazin-1-yl)phenyl)amino)-4-(tetrahydro-2H-pyran-3-yl)amino)-7H-pyrrolo[2,3-d]pyrimidin-5-yl)methanone, (4-fluorophenyl)(4-(((1s,4s)-4-(hydroxymethyl)cyclohexyl)amino)-2-((4-(4-methylpiperazin-1-yl)phenyl)amino)-7H-pyrrolo[2,3-d]pyrimidin-5-yl)methanone, (4-fluorophenyl)(2-((4-(4-methylpiperazin-1-yl)phenyl)amino)-4-(1-(tetrahydro-2H-pyran-4-yl)piperidin-4-yl)amino)-7H-pyrrolo[2,3-d]pyrimidin-5-yl)methanone, (4-((1-(cyclopropylsulfonyl)piperidin-4-yl)amino)-2-((4-(4-methylpiperazin-1-yl)phenyl)amino)-7H-pyrrolo[2,3-d]pyrimidin-5-yl)(4-fluorophenyl)methanone, 1-(4-((5-(4-fluorobenzoyl)-2-((4-(4-methylpiperazin-1-yl)phenyl)amino)-7H-pyrrolo[2,3-d]pyrimidin-4-yl)amino)piperidin-1-yl)-2,2-dimethylpropan-1-one, (S)-(4-fluorophenyl)(2-((4-(4-methylpiperazin-1-yl)phenyl)amino)-4-(tetrahydrofuran-3-yl)amino)-7H-pyrrolo[2,3-d]pyrimidin-5-yl)methanone, (4-fluorophenyl)(2-((4-(4-methylpiperazin-1-yl)phenyl)amino)-4-(oxetan-3-ylamino)-7H-pyrrolo[2,3-d]pyrimidin-5-yl)methanone, (4-fluorophenyl)(4-(((1r,3s,5R,7S)-3-hydroxyadamantan-1-yl)amino)-2-((4-(4-methylpiperazin-1-yl)phenyl)amino)-7H-pyrrolo[2,3-d]pyrimidin-5-yl)methanone, (S)-(4-fluorophenyl)(4-((1-hydroxy-4-methylpentan-2-yl)amino)-2-((4-(4-methylpiperazin-1-yl)phenyl)amino)-7H-pyrrolo[2,3-d]pyrimidin-5-yl)methanone, (R)-(4-fluorophenyl)(4-(1-hydroxy-3,3-dimethylbut-2-yl)amino)-2-((4-(4-methylpiperazin-1-yl)phenyl)amino)-7H-pyrrolo[2,3-d]pyrimidin-5-yl)methanone, (4-fluorophenyl)(2-((4-(4-methylpiperazin-1-yl)phenyl)amino)-4-((tetrahydro-2H-pyran-4-yl)methyl)amino)-7H-pyrrolo[2,3-d]pyrimidin-5-yl)methanone, (4-fluorophenyl)(4-((1-(hydroxymethyl)cyclopropyl)amino)-2-((4-(4-methylpiperazin-1-yl)phenyl)amino)-7H-pyrrolo[2,3-d]pyrimidin-5-yl)methanone, (4-fluorophenyl)(4-((1r,4r)-4-methoxycyclohexyl)amino)-2-((4-(4-methylpiperazin-1-yl)phenyl)amino)-7H-pyrrolo[2,3-d]pyrimidin-5-yl)methanone, (4-fluorophenyl)(2-((4-(4-methylpiperazin-1-yl)phenyl)amino)-4-((2-morpholinoethyl)amino)-7H-pyrrolo[2,3-d]pyrimidin-5-yl)methanone, (4-((2-(dimethylamino)ethyl)amino)-2-((4-(4-methylpiperazin-1-yl)phenyl)amino)-7H-pyrrolo[2,3-d]pyrimidin-5-yl)(4-fluorophenyl)methanone, (4-fluorophenyl)(2-((4-(4-methylpiperazin-1-yl)phenyl)amino)-4-((2-(piperidin-1-yl)ethyl)amino)-7H-pyrrolo[2,3-d]pyrimidin-5-yl)methanone, (2-((3-fluoro-4-(4-methylpiperazin-1-yl)phenyl)amino)-4-(((1r,4r)-4-(hydroxymethyl)cyclohexyl)amino)-7H-pyrrolo[2,3-d]pyrimidin-5-yl)(4-fluorophenyl)methanone, (4-fluorophenyl)(4-(((1r,4r)-4-(hydroxymethyl)cyclohexyl)amino)-2-((2-methoxy-4-(4-methylpiperazin-1-yl)phenyl)amino)-7H-pyrrolo[2,3-d]pyrimidin-5-yl)methanone, (2-((3-fluoro-4-morpholinophenyl)amino)-4-(((1r,4r)-4-(hydroxymethyl)cyclohexyl)amino)-7H-pyrrolo[2,3-d]pyrimidin-5-yl)(4-fluorophenyl)methanone, (4-fluorophenyl)(4-((1-isopropylpiperidin-4-yl)amino)-2-((4-(4-methylpiperazin-1-yl)phenyl)amino)-7H-pyrrolo[2,3-d]pyrimidin-5-yl)methanone, (4-fluorophenyl) (2-((4-(4-methylpiperazin-1-yl)phenyl)amino)-4-((1-methylpiperidin-4-yl)amino)-7H-pyrrolo[2,3-d]pyrimidin-5-yl)methanone, (4-fluorophenyl)(4-((3-(hydroxymethyl)phenyl)amino)-2-((4-(4-methylpiperazin-1-yl)phenyl)amino)-7H-pyrrolo[2,3-d]pyrimidin-5-yl)methanone, (2-((4-((2-(dimethylamino)ethyl)(methyl)amino)-2-methoxyphenyl)amino)-4-((1r,4r)-4-(hydroxymethyl)cyclohexyl)amino)-7H-pyrrolo[2,3-d]pyrimidin-5-yl)(4-fluorophenyl)methanone, (2-((4-(2-(dimethylamino)ethoxy)-2-methoxyphenyl)amino)-4-((1r,4r)-4-(hydroxymethyl)cyclohexyl)amino)-7H-pyrrolo[2,3-d]pyrimidin-5-yl)(4-fluorophenyl)methanone, (4-(((1r,4r)-4-aminocyclohexyl)amino)-2-(4-(4-methylpiperazin-1-yl)phenyl)amino)-7H-pyrrolo[2,3-d]pyrimidin-5-yl)(4-fluorophenyl)methanone, (4-fluorophenyl)(4-(((1r,4r)-4-(hydroxymethyl)cyclohexyl)amino)-2-((1-(1-methylpiperidin-4-yl)-1H-pyrazol-4-yl)amino)-7H-pyrrolo[2,3-d]pyrimidin-5-yl)methanone, (3,4-difluorophenyl)(4-((1r,4r)-4-(hydroxymethyl)cyclohexyl)amino)-2-((4-morpholinophenyl)amino)-7H-pyrrolo[2,3-d]pyrimidin-5-yl)methanone, (2-((4-(2-(dimethylamino)ethoxy)-2-methoxyphenyl)amino)-4-(((1r,4r)-4-(hydroxymethyl)cyclohexyl)amino)-7H-pyrrolo[2,3-d]pyrimidin-5-yl)(4-fluorophenyl)methanone, (4-fluorophenyl)(4-((3R,6S)-6-(hydroxymethyl)tetrahydro-2H-pyran-3-yl)amino)-2-((4-(4-methylpiperazin-1-yl)phenyl)amino)-7H-pyrrolo[2,3-d]pyrimidin-5-yl)methanone, (4-fluorophenyl)(4-(((1r,4r)-4-(hydroxymethyl)cyclohexyl)amino)-2-((3-methoxy-1-methyl-1H-pyrazol-4-yl)amino)-7H-pyrrolo[2,3-d]pyrimidin-5-yl)methanone, ((1r,4r)-4-((2-((4-morpholinophenyl)amino)thieno[2,3-d]pyrimidin-4-yl)amino)cyclohexyl)methanol, (4-(((1r,4r)-4-(hydroxymethyl)cyclohexyl)amino)-2-((4-morpholinophenyl)amino)-7H-pyrrolo[2,3-d]pyrimidin-5-yl)(p-tolyl)methanone, (4-(((1r,4r)-4-(hydroxymethyl)cyclohexyl)amino)-2-((4-morpholinophenyl)amino)-7H-pyrrolo[2,3-d]pyrimidin-5-yl)(phenyl)methanone, (4-fluorophenyl)(4-((1-methylpiperidin-4-yl)amino)-2-((4-morpholinophenyl)amino)-7H-pyrrolo[2,3-d]pyrimidin-5-yl)methanone, (4-fluorophenyl)(4-(((1s,4s)-4-(hydroxymethyl)cyclohexyl)amino)-2-((4-morpholinophenyl)amino)-7H-pyrrolo[2,3-d]pyrimidin-5-yl)methanone,(2-((4-(4-ethylpiperazin-1-yl)phenyl)amino)-4-(((1s,4s)-4-(hydroxymethyl)cyclohexyl)amino)-7H-pyrrolo[2,3-d]pyrimidin-5-yl)(4-fluorophenyl)methanone, (2-chloro-4-fluorophenyl)(4-(((1r,4r)-4-(hydroxymethyl)cyclohexyl)amino)-2-((4-morpholinophenyl)amino)-7H-pyrrolo[2,3-d]pyrimidin-5-yl)methanone, (4-fluorophenyl)(4-(((1s,4s)-4-(hydroxymethyl)cyclohexyl)amino)-2-((1-(1-methylpiperidin-4-yl)-1H-pyrazol-4-yl)amino)-7H-pyrrolo[2,3-d]pyrimidin-5-yl)methanone, (4-fluorophenyl)(4-(((1s,4s)-4-(hydroxymethyl)cyclohexyl)amino)-2-((3-methoxy-1-methyl-1H-pyrazol-4-yl)amino)-7H-pyrrolo[2,3-d]pyrimidin-5-yl)methanone, and (4-fluorophenyl)(4-((1R,4S)-4-hydroxymethyl)cyclopent-2-en-1-yl)amino)-2-((4-(4-methylpiperazin-1-yl)phenyl)amino)-7H-pyrrolo[2,3-d]pyrimidin-5-yl)methanone.

12. A method for synthesizing the compound of formula I according to claim 1, wherein the method is as follows: 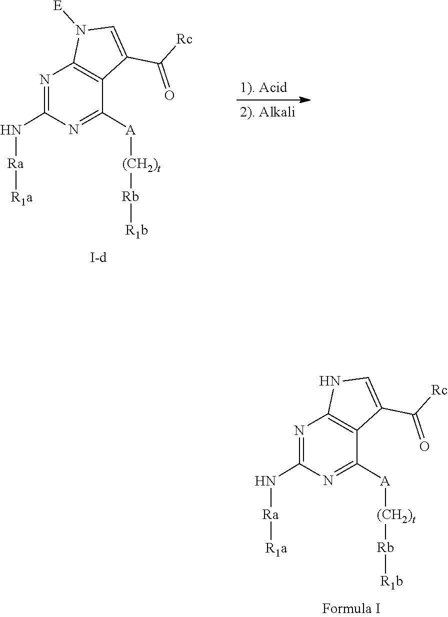
1) removing the E group from the compound I-d under the acid condition, wherein the E is selected from one of trimethylsiloxyethyl methyl, benzyloxycarbonyl, p-toluenesulfonyl, benzenesulfonyl, acetyl, trifluoroacetyl, fluorenylmethoxycarbonyl, and benzyl; and
2) neutralizing the product obtained in step 1) under the alkali condition to obtain the compound of formula I.

13. The method for preparing the compound of formula I according to claim 12, wherein a method for preparing the I-d is as follows: 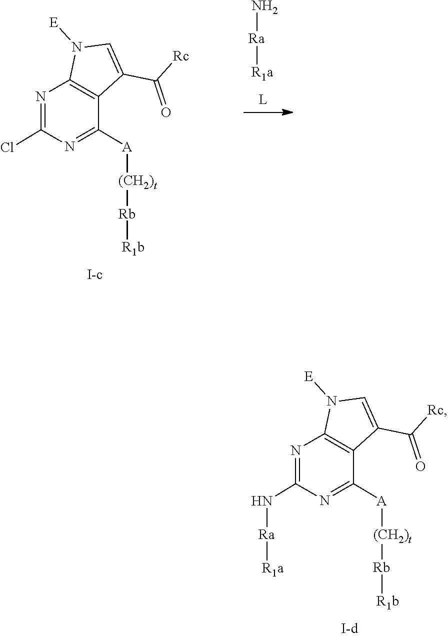
reacting the compound I-c with the amine L under the heating condition to generate the compound I-d.

14. The method for preparing the compound of formula I according to claim 13, wherein a method for preparing the I-c is as follows: 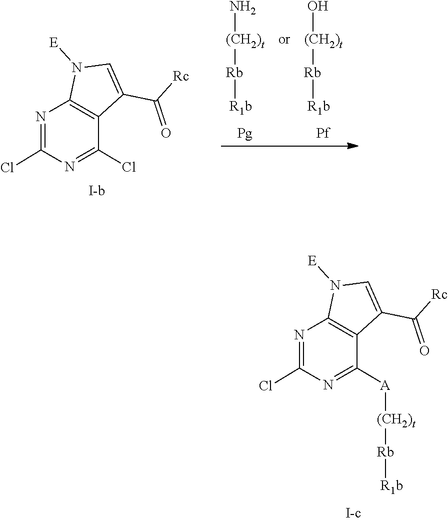
reacting the compound I-b with the amine Pg or the alcohol Pf under the heating condition to generate the compound I-c, wherein preferably, the heating is performed at temperature of 80-100° C.

15. The method for preparing the compound of formula I according to claim 14, wherein a method for preparing the I-b is as follows: 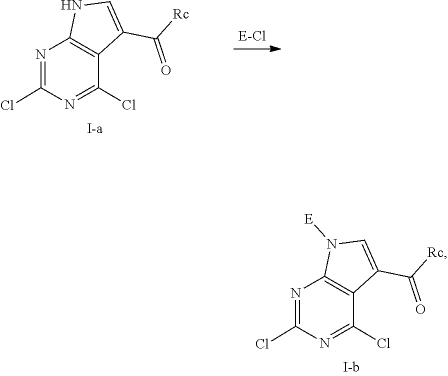
reacting the compound I-a with the E-Cl under the heating condition to generate the compound I-b, wherein preferably, the heating is performed at temperature of 30-60° C.

16. The method for preparing the compound of formula I according to claim 15, wherein a method for preparing the I-a is as follows: 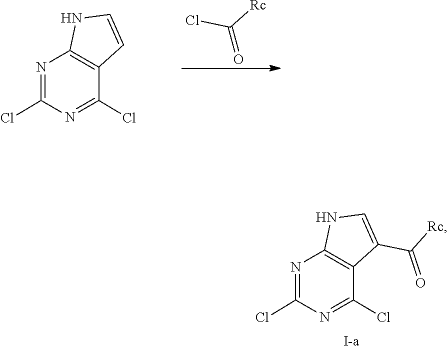
reacting the 2,4-dichloro-7H-pyrrolo[2,3-d]pyrimidine with the 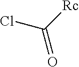
under the ice bath condition to obtain the compound I-a.

17. A pharmaceutical composition comprising the compound or the pharmaceutically acceptable salt thereof according to claim 1, and one or more pharmaceutically acceptable pharmaceutical excipients.

18.-29. (canceled)

30. A method for preparing an oral formulation or a parenteral formulation, wherein the oral formulation is a tablet, a pill, a granule, a powder, a capsule, a syrup, an emulsion, and a microemulsion; and the parenteral formulation is an injection, comprising preparing the oral formulation or a parenteral formulation from the compound or the pharmaceutically acceptable salt thereof according to claim 1.

31. A method for regulating and controlling an EGFR tyrosine kinase activity or preventing and treating an EGFR-related disease, comprising administering the compound or the pharmaceutically acceptable salt thereof according to claim 1 to the subject in need thereof.

32. The method according to claim 31, wherein the regulating and controlling an EGFR tyrosine kinase activity or preventing and treating an EGFR-related disease comprises tumor, diabetes, atherosclerosis, immune system diseases, neurodegenerative diseases, or cardiovascular diseases;
preferably, the tumor is selected from lung cancer, liver cancer, ascitic tumor, metastatic encephaloma, colon cancer, pancreatic cancer, breast cancer, prostate cancer, brain cancer, ovarian cancer, cervical cancer, testicular cancer, kidney cancer, head and neck cancer, lymphoma, melanoma, leukemia, esophageal cancer, stomach cancer, skin cancer, rectal cancer, adrenal cancer, thyroid tumor, and complications thereof;
further preferably, the tumor is selected from lung cancer, liver cancer, breast cancer, ascitic tumor, pancreatic cancer, and metastatic encephaloma;
further preferably, the lung cancer is selected from non-small cell lung cancer or small cell lung cancer;
further preferably, the non-small cell lung cancer is selected from lung adenocarcinoma, lung squamous cell carcinoma, or large cell lung cancer;
further preferably, the non-small cell lung cancer is lung adenocarcinoma;
alternatively, the lung cancer is selected from EGFR-mutant lung cancer, KRAS-mutant lung cancer, HER2-mutant lung cancer, PIK3CA-mutant lung cancer, BRAF-mutant lung cancer, MET gene-mutant lung cancer, ALK gene rearrangement lung cancer, ROS1 gene rearrangement lung cancer, and RET gene rearrangement lung cancer;
preferably, the EGFR-mutant lung cancer is selected from EGFR-Del19-mutant lung cancer, EGFR-L858R-mutant lung cancer, EGFR-C797S-mutant lung cancer, EGFR-C797S/T790M-mutant lung cancer, EGFR-L858R/T790M-mutant lung cancer, EGFR-Del19/T790M-mutant lung cancer, EGFR-L858R/T790M/C797S-mutant lung cancer, EGFR-Del19/T790M/C797S-mutant lung cancer, EGFR D770-N771 ins NPG-mutant lung cancer, or EGFR D770-N771 ins NPG/T790M-mutant lung cancer;
further preferably, non-small cell lung cancer of the EGFR-mutant lung cancer is EGFR Del19/T790M/C797S-mutant lung adenocarcinoma; or
the non-small cell lung cancer of the EGFR-mutant lung cancer is selected from EGFR D770-N771 ins NPG mutation or EGFR D770-N771 ins NPG/T790M mutation.
